SEQUENCE LISTING (7 sequences)

SEQ ID NO: 1 - DNA, 22 bp, Unknown
  IL-6 forward
tacaatgagctgcgtgtggccc

SEQ ID NO: 2 - DNA, 22 bp, Unknown
  IL-6 reverse
aggatggcgtgagggagagcat

SEQ ID NO: 3 - DNA, 20 bp, Unknown
  VEGF forward
ttcgggaaccagacctctca

SEQ ID NO: 4 - DNA, 20 bp, Unknown
  VEGF reverse
aatggcgaatccagtcccac

SEQ ID NO: 5 - DNA, 20 bp, Unknown
  Beta-actin forward
ccggagaggagacttcacag

SEQ ID NO: 6 - DNA, 20 bp, Unknown
  Beta-actin reverse
tccacgatttcccagagaac

SEQ ID NO: 7 - protein, 38 aa, Homo Sapiens
LLGDFFRKSKEKIGKEFKRIVQRIKDFLRNLVPRTESC

(19) United States
(12) Patent Application Publication
(10) Pub. No.: US 2018/0264134 A1
(43) Pub. Date: Sep. 20, 2018

(54) A COMPOSITION OF GOLD NANOPARTICLES HAVING ANTIMICROBIAL PEPTIDES BOUND ON THEIR SURFACE FOR USE THE TREATMENT OF WOUND HEALING OR IN THE TREATMENT OF ISCHEMIC OR VASCULAR DISEASES OR IN THE TREATMENT OF SKIN DISORDERS

(71) Applicant: CENTRO DE NEUROCIENCIAS E BIOLOGIA CELULAR, Coimbra (PT)

(72) Inventors: Lino DA SILVA FERREIRA, COIMBRA (PT); Michela COMUNE, QUARTU SANT-ELENA (IT)

(21) Appl. No.: 15/768,737

(22) PCT Filed: Oct. 17, 2016
(86) PCT No.: PCT/IB2016/056231

§ 371 Date: Apr. 16, 2018

(30) Foreign Application Priority Data
Oct. 15, 2015 (PT) 108893

(51) Int. Cl. A61K 47/69; A61K 35/12; A61P 17/02; A61P 31/00; A61K 35/44; A61K 35/36; A61K 47/52; A61K 38/17

(52) U.S. Cl. CPC A61K 47/6923; A61K 35/12; A61P 17/02; A61P 31/00; A61K 38/1709; A61K 35/36; A61K 47/52; A61K 47/6929; A61K 35/44

(57) ABSTRACT

The present disclosure relates to an antimicrobial peptide conjugated with nanoparticles, in particular the antimicrobial peptide LL37-conjugated gold nanoparticles for use in medicine or cosmetic, for human or animals, namely in the treatment of wound healing or in the treatment of ischemic or vascular diseases, comprising a gold nanoparticle and a plurality of LL-37 peptides, wherein the plurality of LL-37 peptides is bound to the gold nanoparticle surface.

### TECHNICAL DOMAIN

The present disclosure relates to an antimicrobial peptide conjugated with nanoparticles, in particular the antimicrobial peptide LL37-conjugated gold nanoparticles for use in medicine or cosmetic, for human or animals, namely in the treatment of wound healing or in the treatment of ischemic or vascular diseases.

### TECHNICAL BACKGROUND

Topical therapies that combine antimicrobial and pro-regenerative effects are of great potential in the context of skin wound healing. Antimicrobial peptides (AMPs) act as a first line of defence in the human body against bacteria, virus and fungi, and some of them modulate regeneration processes⁴. In the last years, these small peptides (typically below 40 aminoacids) have been tested as a potential anti-infective therapy, at least for some indications, and thus an alternative to conventional antibiotics. Currently, there are 10 AMP compounds in clinical trials, mostly for skin applications. Two AMPs are in phase 3 clinical trials (to treat C. difficile infections and diabetic foot ulcers). Some of these AMPs combine antimicrobial properties (targeting microorganisms) with immunomodulatory, pro-angiogenic and tissue regenerative properties (both targeting human cells). For example, LL37 (one of the AMPs currently in clinical trials⁶) is an AMP predominantly found in human skin that acts at different levels of skin homeostasis. LL37 is a chemoattractant of mast cells, monocytes, T lymphocytes and neutrophils, and regulates inflammation, angiogenesis and wound healing. The wound healing properties of LL37 peptide (either in a soluble formulation or in a formulation consisting in polymeric nanoparticles releasing LL37) has been demonstrated in wound animal models, as well as in a recent phase I/II clinical trial⁶.

Antimicrobial peptides (AMPs) are a class of biomolecules present in large number in nature (more than 800 sequences), which are effective against several strains of bacteria, fungi and viruses, but also may mediate important biological reactions such as angiogenesis, regeneration, chemotaxis, immunomodulation, among others. Several studies have explored the physical immobilization of AMPs in nanoparticles (NPs) to prolong their biological activity; however, this approach may be insufficient to yield high local density of peptide to potentiate their biological activity. Recently, an alternative approach has been described base on AMP-conjugated NPs. In this case, the AMP is chemically bond to the NP. AMP-conjugated NPs may offer higher stability against enzymes, enhanced antimicrobial properties (due to an increase in the local density of positive charges and peptide mass) and improved targeting compared with free AMP. These formulations have been evaluated in pre-clinical animal tests for the treatment of meningitis. However, so far, no AMP-immobilized NPs have been tested in the context of their regenerative potential, including angiogenic and wound healing properties both in vitro or in vivo. The conjugation of AMPs to NPs may offer a prolonged activation of signalling cascades and thus potentiate their regeneration potential.

LL37 is an antimicrobial peptide (AMP) predominantly found in human skin that acts at different levels for the homeostasis of the skin. LL37 acts as first line of defence against bacteria, virus and fungi. Importantly, LL37 plays an important role in immunomodulation, angiogenesis and wound healing properties. It has been shown that LL37 is a chemoattractant for mast cells, monocytes, T lymphocytes and neutrophils. Furthermore, they contribute to regulation of inflammation and promote wound healing and angiogenesis. Angiogenic properties of LL37 have been associated with binding via formyl peptide receptor-like 1 (FPRL1) in endothelial cells, while the immunomodulatory and chemotactic properties have been associated with binding via P2X7, epidermal growth factor receptor (EGFR) or FPRL1. Although the wound healing properties of LL37 peptide have been demonstrated, only recently the peptide has been immobilized on NPs.

These facts are disclosed in order to illustrate the technical problem addressed by the present disclosure.

### GENERAL DESCRIPTION

One of the approaches being tested to administer AMPs in vivo and prevent their potential toxicity while increasing their stability against protease degradation and serum inactivation is through the chemical immobilization of AMPs in nanoparticles (NPs). It was been recently demonstrated that AMP-conjugated NPs may offer higher stability, lower toxicity, enhanced antimicrobial properties (due to an increase in the local density of positive charges and peptide mass) and improved targeting compared with free AMP. However, AMP-conjugated NPs have not been tested either in vitro or in vivo in the context of their regenerative potential relatively to soluble AMPs. It is unclear whether multivalent AMP-conjugated NPs may modulate cellular signalling and bioactivity. Although it has been demonstrated that in some cases multivalent ligand-containing NPs have superior bioactivity than soluble ligands, such activity profile are likely dependent in the type of cellular target (e.g. receptor, protein, ionic channel) and type of cell.

The present disclosure concerns the investigation of the wound healing potential of LL37-conjugated NPs both in vitro (migration of keratinocytes) and in vivo (skin wound healing). These NPs have a gold (Au) core and a hydrophilic cationic LL37 peptide shell. The gold nanoparticles (AuNPs) were selected because it is relatively easy the modification of their properties (e.g. size), the immobilization of high concentrations of AMPs per surface area, and they have a biomedical track. LL37-conjugated NPs were prepared by a one-step procedure. Initially, the physico-chemical properties of LL37-gold nanoparticle (LL37-AuNPs) were characterized. To show the unique properties of LL37-AuNPs, their pro-migratory properties against keratinocytes were evaluated, an important player in the context of skin healing, and evaluated their mechanism relatively to LL37 peptide. Finally, it was evaluated in vivo the regenerative potential of LL37 and LL37-AuNPs in a splinted mouse full thickness excisional model. Overall the results indicate that LL37-conjugated NPs have enhanced wound-healing properties than LL37 peptide because they prolong in time the biological activity of the peptide.

One aspect of the present disclosure is related to a composition for use in the treatment of wound healing, or in the treatment of ischemic or vascular diseases, comprising: a gold nanoparticle and a plurality of LL-37 peptides, wherein the plurality of LL-37 peptides is bound to the gold nanoparticle surface; namely as an enhancer of the treatment of wound healing, or in the treatment of ischemic or vascular diseases.

In an embodiment, the wound healing potential of LL37-conjugated NPs that have a gold (Au) core and a hydrophilic cationic LL37 peptide shell were investigated. The Au NPs were selected because it is relatively easy the immobilization of high concentrations of LL37 per surface area, the modification of their properties, including size, charge and morphology, and they have been used in the clinic for many years. The production of nanoparticulate formulation now disclosed involved only a processing step, which included the mixing of the peptide with gold salts, at room temperature, for 1 day followed by a centrifugation step. This process makes possible the large-scale production of LL37-conjugated NPs at a relative low cost. Furthermore, LL37-conjugated NPs have enhanced wound-healing properties than LL37 peptide because they prolong in time the biological activity of the peptide. So far no study has compared the in vivo performance of soluble AMP relatively to chemically immobilized AMP formulations in terms of their regenerative potential.

Therefore, the present disclosure discloses:
the physico-chemical properties of LL37-AuNPs;
the binding process of LL37 peptide to the NP formulation by molecular dynamic studies;
the cytotoxicity properties against endothelial cells and keratinocytes;
the antimicrobial properties as well as their stability against proteases.

To show the unique properties of the formulation now disclosed, the evaluation of their pro-migratory properties against keratinocytes and the pro-angiogenic properties against endothelial cells and a chorioallantoic membrane (CAM) assay was also conducted. The evaluation of the regenerative potential of LL37-Au NPs in a splinted mouse full thickness excisional model was conducted in vivo.

Furthermore, the regenerative potential of a formulation such as the one now disclosed was not previously demonstrated.

The antimicrobial peptide (AMP) LL37 combines antimicrobial with pro-regenerative properties and thus represents a promising topical therapy to address both problems. Here, it is investigated the wound healing potential of LL37 peptide and LL37-conjugated nanoparticles (LL37-AuNPs) both in vitro (migration of keratinocytes) and in vivo (skin wound healing). The results show that LL37-conjugated NPs, but not LL37 peptide, have the capacity to prolong the phosphorylation of EGFR and ERK1/2 and enhance the migratory properties of keratinocytes in a large in vitro wound model. It is further reported that both LL37 and LL37-conjugated NPs promote keratinocyte migration by the transactivation of EGFR, a process that seems to be initiated at the P2X7 receptor, as confirmed by chemical and genetic inhibition studies. Finally, it is show in vivo that LL37-conjugated NPs have higher wound healing activity than LL37 peptide in a splinted mouse full thickness excisional model. Animal wounds treated by LL37-conjugated NPs have higher expression of collagen, IL6 and VEGF than the ones treated with LL37 peptide or NPs without LL37. Altogether, the conjugation of AMPs to NPs offers a promising platform to enhance their pro-regenerative properties.

To show the unique properties of LL37-Au NPs it was evaluated their pro-migratory properties against keratinocytes, an important player in the context of skin healing, and evaluated their mechanism relatively to LL37 peptide. Finally, it was evaluated the in vivo the regenerative potential of LL37 and LL37-Au NPs in a splinted mouse full thickness excisional model. Overall the results indicate that LL37-conjugated NPs have enhanced wound-healing properties than LL37 peptide because they prolong in time the biological activity of the peptide.

In the present disclosure it is show that AMP-nanoscale therapeutic formulation with high skin regenerative potential, obtained in a rapid one-step synthetic process. LL37-Au NPs have higher in vitro (keratinocyte migration assay) and in vivo (skin wound healing assay) bioactivity than soluble LL37 peptide. The enhanced activity of LL37-Au NPs relatively to LL37 peptide is due to a prolonged activation of EGFR in keratinocytes, likely due to the retention of the phosphorylated EGFR at the plasma membrane preventing EGFR dephosphorylating.

The present disclosure relates to a composition for use in medicine or veterinary comprising a gold nanoparticle and a plurality of LL-37 peptides as antimicrobial peptide, wherein the plurality of LL-37 peptides is bound to the gold nanoparticle surface.

The present disclosure also relates to said composition for use in a method of treating or in therapy of wounds, or ischemic diseases, or vascular diseases, or skin disorders.

In an embodiment, the LL-37 may be bound to the gold nanoparticle surface by covalent bonds.

In an embodiment, the composition may further comprises a buffer, wherein said buffer is selected from the following list: TAPS, Bicine, Tris, Tricine, TAPSO, HEPES, TES, MOPS, PIPES, Cacodylate, SSC, MES, and combinations thereof.

In an embodiment, the pH of the composition may be between 4.5-8, preferably 5-7.5, more preferably between 5-6.

In an embodiment, the ratio LL-37:gold nanoparticle may be 3:1-1:2, preferably 2:1-1:1. The ratio is given in weight by weight; volume by volume or in number of LL37 by number of gold nanoparticles.

In an embodiment, LL-37 antimicrobial peptide concentration may be between 0.20-0.10 mM, preferentially between 0.3-0.5 mM.

In an embodiment, the composition may further comprise stem cells.

In an embodiment, the composition may further comprise endothelial cells, namely human umbilical vein endothelial cells, or keratinocytes, and combinations thereof.

In an embodiment, the composition may be a topic formulation or an injectable formulation.

In an embodiment, the composition may be for use in the treatment of wounds in diabetic mammals.

In an embodiment, the composition may be for the use in the treatment of eczema or psoriasis.

The present disclosure also relates to the use of the composition as an enhancer in the treatment of wound healing.

Throughout the description and claims the word “comprise” and variations of the word, are not intended to exclude other technical features, additives, components, or steps. Additional objectives, advantages and features of the solution will become apparent to those skilled in the art upon examination of the description or may be learned by practice of the solution.

### DESCRIPTION OF THE DRAWINGS

The following figures provide preferred embodiments for the present disclosure and should not be seen as limiting the scope of the disclosure.

FIG. 1: (a) Schematic representation of Au NP synthesis in the presence of Au ions, HEPES buffer and LL37 peptides. (b) UV-vis spectra of AuNPs synthesized using citrate reduction method and of LL-37 AuNPs with HEPES pH 5, pH 6, pH 7.5 with one step synthesis procedure. (c) UV-vis kinetic studies of the synthesis of LL-37 AuNPs under pH 5, pH 6 and pH 7.5. (d) TGA and (e) FTIR spectra of LL-37 peptide and LL-37 AuNPs synthesized at different pHs. (f) The representative TEM images of citrate reduced AuNPs (f1) and LL-37 AuNPs synthesized at pH 5 (f2), 7.5 (f3) and 6 (f4). (g1, g2, g3, g4) Particle size analysis of LL-37 AuNPs synthesized at different pHs and (g5) Percentage of NPs with a circular or elongated morphology during the growth of LL37 Au NPs.

FIG. 2: (a) Distance of the residues to the Au surface at the end of the simulation. The line at 0.3 nm represents the cut-off used to define direct contact between the atoms of the amino acids and the Au surface. (b) Evolution of: (b1) the radius of gyration (Rg) and (b2) RMSD during the simulation for the investigated systems. Each curve represents an independent system. (b3) Evaluation of the evolution in time of the secondary structure of each peptide.

FIG. 3: Cell viability (a1) and ATP production (a3) studies after 5 h of LL37, LL37 AuNPs, AuNPs incubation in HaCaT. Cell viability (a2) and ATP production (a4) studies after 48 h of LL37, LL37 AuNPs, AuNPs incubation in HaCaT. Cell viability (b1) and ATP production (b3) studies after 5 h of LL37, LL37 AuNPs, AuNPs incubation in HUVECs. Cell viability (b2) and ATP production (b4) studies after 48 h of LL37, LL37 AuNPs, AuNPs incubation in HUVECs. (c) ICP-MS study of internalization LL37 AuNPs and AuNPs by HaCaT and HUVEC at 5 h (c1 and c2) and 48 h (c3 and c4). (d) Internalization mechanism of LL37 AuNPs: (d1) Uptake of LL37 AuNPs by HaCaT in the presence of several endocytosis inhibitors and (d2) after knocking down siRNA for specific endocytosis pathways.

FIG. 4: Intracellular localization of LL37 AuNPs as assessed by confocal microscopy. (a) Cells were exposed for 30 min, 4 h and 24 h to fluorescently labeled LL37 Au NPs (200 μg/mL). At the end of each time, HaCaT were washed with PBS, fixed and stained for endolysomal compartment marker as early endosome EEA1 (cell membrane was stained against EGFR). Confocal microscopy was performed to identify the exact intracellular location of the NPs following internalization. (b) Characterization of LL37 AuNPs intracellular trafficking in HaCaT by transmission electron microscopy (TEM). TEM results confirm that NPs are taken up by HaCaT and localize within endolysosomal structures after 30 min (b1) and 4 h (b2) of internalization.

FIG. 5: Cell migration studies by quantification of area closure in a scratch assay. Wound area at 48 h post treatment was normalized by initial wound area. Results are average±SEM, n=6. Scratch assay of HaCaT cells after 48 h incubation with LL37 peptide (1 μg/mL), AuNPs (200 μg/mL) and LL37 AuNPs (200 μg/mL) performed in the presence of several chemical antagonist for (a1) FPRL1 receptor, (a2) EGFR receptor, (a3) ADAM metalloproteases, (a4) for purinergic receptors and specifically for P2X7 receptor. Scratch assay of HaCaT cells after 48 h incubation with LL37 peptide (1 μg/mL), AuNPs (200 μg/mL) and LL37 AuNPs (200 μg/mL) performed after siRNA knockdown for ADAM 17 (a5) and P2X7 receptor (a6).

FIG. 6: Bioactivity of LL37 AuNPs as assessed by their ability to prolong the activation of EGFR and ERK pathway, prolong HaCaT migration and to be resistant to proteases. Phosphorylation profile (phospho/total protein) of (a) EGFR and (b) ERK pathway over time mediated by LL37 (5 μg/mL), LL37AuNPs (200 μg/mL) and AuNPs (200 μg/mL). (c) Scratch assay of HaCaT cells after 96 h of incubation with LL37 peptide (1 μg/mL), AuNPs (15 μg/mL) and LL37 AuNPs (15 μg/mL); (d) Scratch assay of HaCaT cells mediated by soluble LL37 and LL37 Au NPs previously incubated with several concentration of Trypsin (0.010, 0.100, 1 and 100 μM) for 2 hours at 37° C. LL37 nanoparticles were then centrifuged, resuspended and used to perform the in vitro scratch assay in HaCaT.

FIG. 7: In vivo wound healing properties of LL37-Au NPs: granulation tissue and inflammation. (a) Wound closure in wounds treated with vehicle (0.9% NaCl), LL37 peptide (70 μg per wound), Au NPs (200 μg per wound) or LL37-Au NPs (200 μg per wound). The formulations were administered intradermally at several sites around the wound. Ten animals (therefore 20 wounds) were used per each group. Wound areas were quantified by a high definition camera. Results are average±SEM, n=20. (b) Quantification of myeloperoxidase (MPO) activity at days 5 and 10. Results are average±SEM, n=10. (c) Quantification of collagen at days 5 and 10 by a sircol assay. Results are average±SEM, n=10. (d) Quantification of IL6 by qRT-PCR at days 5 and 10. Results are average±SEM, n=10. (e) Histological analysis (Hematoxylin & Eosin staining and Masson trichrome staining) of wound tissues at day 5 and 10 of treatment. In graphs a, b, c and d, *P<0.05, **P<0.01, ***P<0.001 and ****P<0.0001 indicates statistical significance between treatment groups. In graph a statistical significance is against non-treated animals.

FIG. 8: (a) Fluorescence Microscopic Images of entire tissue sections stained for CD31 marker acquired by a scanning MIRAX microscope (Zeiss, USA). (b) Quantification of VEGF expressions after treatment with LL37 peptide, AuNPs and LL37 AuNPs at day 5 and 10. (c) Quantification of CD31 staining. The percentage of red pixel represents both endothelial and red blood cells.

Results were expressed as percentage of red pixels over the total amount of pixels within the analyzed surface.

FIG. 9: Screening conditions for the preparation of LL37 Au NPs: effect of concentration of gold in the growth of LL37-AuNPs. The reactions were performed in HEPES buffer pH 5 in the presence of 0.25 mM LL-37 and three different concentration of Au (0.25 mM, 0.5 mM and 1 mM). (a) TEM images, (b) Particle size analyses, (c.1) the absorbance peak and (d.1) the growth kinetic of Au NPs under different conditions was followed by UV-Vis spectroscopy. The reduction rate was faster increasing Au concentration as shown in FIG. d. However, the fast synthesis, only 30 minutes for the third combination, could be the cause for their not spherical shape and bigger size of compared with the second combination (b.2 and b.3). The absorbance peak of LL-37 AuNPs (1 mM Au) in 550 nm also confirms a bigger size and not homogenous LL37NPs, while LL-37 AuNPs (Au 0.5 mM) showed 530 nm peak, smaller spherical and homogenous NPs. The combination with the lowest Au concentration showed low capacity to create LL37-AuNPs even after 6 days of reaction.

FIG. 10: Screening conditions for the preparation of LL37 Au NPs: effect of concentration of LL-37 in the growth of LL37-AuNPs The reactions were performed in HEPES buffer pH 5 in the presence of 0.5 mM Au and three different concentration of LL-37 (a.4—0.25 mM, a.5—0.5 mM and—a.6 1 mM). a) TEM images, (b.3 and b.4) Particle size analyses, (c.2) the absorbance peak and (d.2) the growth kinetic of Au NPs under different conditions was followed by UV-Vis spectroscopy. The reduction rate was faster for the lowest concentration of LL-37 tested showing smaller, spherical and homogenous LL-37 NPs after 1 day of reaction. The combination with the highest LL-37 concentration showed low capacity to create LL37-AuNPs even after 10 days of reaction, while maximizing also the concentration of Au, nanotriangles, pentagon-shape, nanorods LL-37 AuNPs, were obtained after 1 day of reaction.

FIG. 11: MD trajectories for the interaction of one Au NP with six LL37 peptides. The trajectories for averaged distances of each amino acids residue of LL37 to the AuNPs surface are represented.

FIG. 12: Stability of NPs suspended in culture medium (a) Zeta potential of LL37 Au NPs (a.1) and Au NPs (a.2) suspended in HEPES, DMEM medium containing 10% (v/v) FBS, or EGM-2 medium containing 2% (v/v) FBS. Diameter and counts (kcps) of LL37 Au NPs and Au NPs in DMEM (medium to culture HaCaT) (b.1) or suspended in EGM-2 (medium to culture HUVECs) (b.2). The average diameter of NPs (200 μg/mL) suspended in 2 mL of cell culture media was determined by a dynamic light scattering method (DLS) using a Zeta Plus analyzer (Brookhaven), from six independent measurements. Average±SEM, n=3.

FIG. 13: Cytotoxicity of soluble LL37 Au NPs, AuNPs and LL37 in HaCaT (a, b), HUVECs (c) and activation of platelets (d). (a, c) Cells (HaCaT and HUVECs), were exposed for 5 and 48 h to different concentrations of soluble LL37, LL37 Au NPs and Au NPs, followed by the quantification of relative membrane potential (% DioC5(3) fluorescence relative to control) in (a) HaCaT and (c) HUVECs. (b) HaCaT were exposed for 5 and 48 h to different concentrations of soluble LL37, LL37 Au NPs and Au NPs, followed by the quantification of ROS species by flow cytometry (d) Activation of platelets by NPs. Human peripheral blood was in contact with NPs or PBS for 1 h and the percentage of activated platelets quantified, using ADP as mild activator. *** means P<0.001 relatively to control.

FIG. 14: Antimicrobial and Pro-angiogenic activity of soluble LL37, LL37 AuNPs and AuNPs. (a) The antimicrobial properties of LL37, LL37 Au NPs and LL37 AuNPs after a sterilization step by autoclave have been assessed against 10⁵ CFU of E. coli. At different time interval, aliquots were taken out and plated on LB-agar plate. Bacteria colonies were counted after 24 h. Results are average±SD, n=3. (b) Secretion of (VEGF165) in HUVECs mediated by LL37 and LL37 Au NPs were assessed by ELISA. The results showed that both LL37 and LL37 Au NPs induce the secretion of VEGF165. (c, d) Pro-angiogenic activity of LL37 and LL37-Au NPs assessed in a chorioallantoic membrane (CAM) assay. Both LL37 and LL37-Au NPs induced vessel growth. In both cases, the mesenchymal region of CAM was dilated and a higher number of blood vessels and inflammatory precursor cells were observed than in control (CAM treated with vehicle) (d, e).

FIG. 15: (a) Toxicity of chemical inhibitors used for NPs internalization studies. HaCaT were exposed for 6 h to chemical inhibitors of several internalization pathways. The quantification of cell viability was assessed by flow cytometry. Results are average±SEM, n=3. (b) Validation of inhibitory activity of Dynosor. To confirm the inhibitory activity of dynasor uptake studies of FITC-labeled transferrin, known to selectively enter cells via clathrin-mediated endocytosis, were performed. HaCaT keratinocyte cells were cultured on 24 well plates and treated or not with dynasor (80 μM, 30 min pre-incubation) followed by addition of 1 μg/mL FITC-labeled transferrin for 3 min at 4° C. Internalization of transferrin was quantified by flow cytometry. (c) Expression of EGFR, FPRL1 and P2X7 receptors in HaCaT. HaCaT were stained for several membrane receptors as EGFR, FPRL1 and P2X7 and their expression were quantified by flow cytometry. Results are average±SEM, n=3.

FIG. 16: Intracellular trafficking of LL37 Au NPs in HaCaT. Cells were exposed for 30 min, 4 h and 24 h to fluorescently labeled LL37 Au NPs (200 μg/mL). At the end of each time, HaCaT were washed with PBS, fixed and stained for endolysomal compartment markers as early endosome EEA1 and late endosome/lysosome marker protein RAB7 (cell membrane was stained against EGFR). Confocal microscopy was performed to identify the exact intracellular location of the NPs following internalization. The results indicate that a significant part of the NPs (ca. 35%) are located in late endosomes after 24 h (a and c), while a 10% of the NPs are located in early endosome (b).

FIG. 17: Intracellular trafficking of soluble LL37 in HaCaT. Cells were exposed for 30 min, 4 h and 24 h to fluorescently labeled LL37 (1 μg/mL). At the end of each time, HaCaT were washed with PBS, fixed and stained for endolysomal compartment markers as early endosome EEA1 (a and b) and late endosome/lysosome marker protein RAB7 (c and d) (cell membrane was stained against EGFR). Confocal microscopy was performed to identify the internalization and intracellular location of soluble LL37. The results indicate any significant colocalization with EEA1 and RAB7 for all the 3 time points studied.

FIG. 18: Intracellular trafficking of LL37 Au NPs in HUVECs. Cells were exposed for 2 h and 4 h to fluorescently labeled LL37 Au NPs (200 μg/mL). At the end of each time, HUVECs were washed with PBS, fixed and stained for endolysomal compartment markers as early endosome EEA1 and late endosome/lysosome marker protein RAB7 (cell membrane was stained against CD31). Confocal microscopy was performed to identify the exact intracellular location of the NPs following internalization. The results indicate that NPs do not colocalize with both early and late endosomes after 2 h and 4 h.

FIG. 19: Expression of EGFR (a.1) and FPRL1 (a.2) receptors in Human Cells. HUVECs, Macrophages, Fibroblasts and HaCat were stained for EGFR and FPRL1 receptors and their expression were quantified by flow cytometry. Results are average±SEM, n=3.

FIG. 20: (a) In vivo wound area closure. Optical images of full-thickness excisional wounds were made using an 8 mm round skin biopsy punch of 6-7 week old RjHan:NMRI female mice. Wound areas were quantified using the Jmicro Vision software up to 10 days. Wound sizes are expressed as percentage of the initial respective wound. (b, c) In vivo distribution of LL37 Au NPs. Au content in different organs of mice, after 48 h, administered by a single (single dose: 1 mg per animal) of LL37 Au NPs, as quantified by ICP-MS at day 2 (b) and at day 15 (c). Results are average±SEM, n=5 (d, e, f) In vivo evaluation of biochemical parameters after 2 days exposure to LL37 Au NPs. Blood Serum collected at day 2 was analyzed for the following biochemical parameters: urea, transaminase-GPT and LDH. This analysis showed no relevant changes compared with untreated animal values demonstrating absence of cytotoxic effect in vivo.

FIG. 21: Zeta potential analyses showed that Au NPs prepared with HEPES at pH 5, 6 and 7.5. The Au NPs showed a SPR band at 720, 650 and 920 nm for pH 7.5, 6 and 5, respectively. TEM analyses showed the formation of irregular shapes Au NPs for pH 6 and 7.5 conditions and of large clusters of heterogeneous and aggregated Au NPs synthesised at pH 5.0.

FIG. 22: Synthesis and characterization of LL37-Au NPs. (a) Schematic representation of Au NP synthesis in the presence of LL37 peptide. (b) Time dependent absorbance for the synthesis of LL37-Au NPs in HEPES buffer pH 5.0 (Au ions=0.5 mM; LL37=0.25 mM). (c) FTIR and (d) TGA analyses of LL37 peptide, Au NPs and LL37-Au NPs. (e) Distance of LL37 aminoacid residues to the Au surface at the end of the simulation. Results are Average±SEM, n=20. The line at 0.3 nm represents the cut-off used to define direct contact between the atoms of the amino acids and the Au surface. (f) Gyration radius profile of LL37 in LL37-Au NPs. Average±SEM, n=20. (g) Time-course of LL37 peptide secondary structure in LL37-Au NPs. Aminoacid residues are identified by numbers that are displayed in figure e.

FIG. 23: Intracellular trafficking of LL37-Au NPs. (a) Intracellular location of LL37-Au NPs in keratinocytes. (a.1) Keratinocytes were exposed to rhodamine-labeled LL37-Au NPs (100 μg/mL) for 4 h. At the end, keratinocytes were washed and fixed before confocal microscopy characterization. Bar corresponds to 15 μm. (a.2) Quantification of the co-location of NPs with EEA1, Rab7 or EGFR. Results are Average±SEM, from 6 different confocal images (40× objective). **P<0.01 and ***P<0.001 indicates statistical significance between groups. (b) Characterization of LL37-Au NPs intracellular trafficking in keratinocytes by transmission electron microscopy (TEM). TEM results confirm that NPs are taken up by keratinocytes and localize within endolysosomal structures after 30 min (b.1), 4 h (b.2) and 24 h (b.3); however, some of the NPs are in contact with the cell membrane at least for 4 h. Arrows indicate sites of internalization. Cell spots defined by a dashed square means areas of magnification. In b.1 and b.2, bar corresponds to 2000 nm (left) and 100 nm (right, magnification). In b.3, bar corresponds to 100 nm.

FIG. 24: Bioactivity of LL37-Au NPs: scratch assay. (a) Schematic representation of the transactivation mechanism of EGFR. (b) Scratch assay. Confluent keratinocytes were starved for 15 h in DMEM with 0.5% FBS and then incubated for 1 h with specific inhibitors followed by the incubation for 5 h with LL37 (1 μg/mL), LL37-Au NPs or Au NPs (both at 200 μg/mL). Cells were then washed with PBS, a scratch was created, the plates re-coated with fibronectin, cells were again washed and maintained in starvation medium up to 72 h. Cell migration was monitored by a high-content microscope. (c) Scratch closure at 48 h post-treatment. Final wound area was normalized by initial wound area. Results are average±SEM, n=6. The following chemical inhibitors have been used: WRW4 as a FPRL1 receptor inhibitor and Erlotinib as EGFR receptor inhibitor. ^{#}P<0.05, ^{##}P<0.01, ^{###}P<0.001 and ^{####}P<0.0001 indicates statistical significance relatively to control (cells without treatment). *P<0.05, **P<0.01, ***P<0.001 and ****P<0.0001 indicates statistical significance between treatment groups.

FIG. 25: Bioactivity of LL37-Au NPs: scratch assay. Scratch closure (at 48 h post-treatment) in keratinocytes treated with inhibitors of metalloproteases (a) and P2X receptors (b) and then exposed to LL37 (1 μg/mL), LL37-Au NPs or Au NPs (both at 200 μg/mL). Final wound area was normalized by initial wound area. Results are average±SEM, n=6. The following chemical inhibitors have been used: Marimastat an ADAM metalloprotease inhibitor, PPADS a generic P2X receptor antagonist and A-740003 a selective P2X7 receptor antagonist. ^{#}P<0.05, ^{##}P<0.01, ^{###}P<0.001 and ^{####}P<0.0001 indicates statistical significance relatively to control (cells without treatment). *P<0.05, **P<0.01, ***P<0.001 and ****P<0.0001 indicates statistical significance between treatment groups.

FIG. 26: Bioactivity of LL37-Au NPs: electrophysiology studies. (a) Representative traces of inward-currents recorded in whole cell patch-clamped HaCaT cells at a holding potential of −70 mV exposed to (a1) Au NPs (200 μg/mL), (a.2) LL37 (1 μg/mL) or (a.3) LL37-Au NPs (200 μg/mL). Forty single cells were exposed twice to the different treatments and the inward currents recorded presented a reproducible ratio between the charge transfer measured in the first 100 s (Q₁₀₀) in the second exposure (S2) over the first exposure (S1) as quantified in (b). The inward currents triggered by Au NPs and LL37-Au NPs, but not by LL37 were attenuated by the presence of apyrase (20 U/mL), as observed by a reduced charge transfer in S2 in the presence of apyrase (darker background), as depicted in the lower traces in (a) and quantified by a reduced S2/S1 ratio shown in (b). Data are presented as mean±SEM of the ratio of Q₁₀₀ in S2 over S1 measured from 4-5 different cells per condition. *P<0.05 unpaired t-test.

FIG. 27: Bioactivity of LL37-Au NPs: molecular studies. (a.1) Phosphorylation profile (phospho/total protein) of EGFR in keratinocytes after contact with LL37 (5 μg/mL), LL37-Au NPs (15 or 200 μg/mL) or Au NPs (200 μg/mL) for a certain time (up to 60 min). (a.2) Phosphorylation profile of EGFR in keratinocytes after contact with LL37-Au NPs (200 μg/mL) with or without washing the LL37-Au NPs. The “10′+20′” on top of the column means that cells were washed after 10 min of contact with LL37-Au NPs and then cultured for more 20 min in medium without NPs. Epidermal growth factor (EGF) has been used as positive control. Results are average±SEM, n=3. (b) Phosphorylation profile (phospho/total protein) of ERK without washing the LL37-Au NPs in contact with keratinocytes. Results are average±SEM, n=3. (c) Scratch assay of keratinocytes after 96 h of incubation with LL37 peptide (1 μg/mL), Au NPs (15 μg/mL) and LL37-Au NPs (15 μg/mL). (c.1) Light microscopy images of the healing of the scratch at 96 h. Bars correspond to 1 mm. (c.2) Quantification of scratch closure at 96 h. Results are average±SEM, n=3. In graphs a, b and c, *P<0.05, **P<0.01 and *** P<0.001 indicate statistical significance between treatment groups.

FIG. 28: In vivo wound healing properties of LL37-Au NPs: neovascularization. Wounds were treated with vehicle (0.9% NaCl), LL37 peptide (70 μg per wound), Au NPs (200 μg per wound) or LL37-Au NPs (200 μg per wound). (a) qRT-PCR VEGF expression in wounds treated with LL37 peptide, LL37-Au NPs or Au NPs at day 5 and 10. Results are average±SEM, n=10. (b.1) Quantification of CD31 staining. The percentage of red pixel represents both endothelial and red blood cells. Results were expressed as percentage of red pixels over the total amount of pixels within the analyzed surface. Results are average±SEM, n=10. (b.2) Representative immunofluorescent images for each experimental group. Bars correspond to 500 μm. **P<0.01 indicates statistical significance between treatment groups (a) or between LL37-Au NPs and non-treated.

FIG. 29: Screening conditions for the preparation of LL37-Au NPs: effect of concentration of Au in the growth of LL37-Au NPs. The reactions were performed in HEPES buffer pH 5 in the presence of 0.25 mM LL37 and three different concentration of Au (0.25 mM, 0.5 mM and 1 mM). (a) Growth kinetic of Au NPs under different conditions, followed by UV-Vis spectroscopy. The reduction rate was faster for reactions having high concentrations of Au. (b) Absorbance peaks of the NPs at the end of the reactions (Au 1 mM: 20 min; Au 0.5 mM: 1 day; Au 0.25 mM: 6 days). The absorbance peak of LL37 Au NPs (1 mM Au) at 550 nm confirms a large diameter and not homogenous LL37-Au NPs. © TEM images and histograms showing the distribution of NP diameters.

FIG. 30: Screening conditions for the preparation of LL37-Au NPs: effect of concentration of LL37 in the growth of LL37-Au NPs. The reactions were performed in HEPES buffer pH 5 in the presence of 0.5 mM Au and three different concentration of LL-37 (0.25 mM, 0.5 mM and 1 mM). (a) Growth kinetic of Au NPs under different conditions, followed by UV-Vis spectroscopy. The reduction rate was faster for the lowest concentration of LL37 tested showing smaller, spherical and homogenous LL37-Au NPs after 1 day of reaction. (b) Absorbance peak of the NPs at the end of the reaction (LL37 1 mM: 10 days; LL37 0.5 mM: 5 days; LL37 0.25 mM: 1 day). (c) TEM images and histograms showing the distribution of NP diameters.

FIG. 31: Screening conditions for the preparation of LL37-Au NPs: effect of pH. The reactions were performed in HEPES buffer pH 5, 6 or 7.5 in the presence of 0.25 mM LL37 and 0.5 mM Au. (a) Growth kinetic of Au NPs under different conditions, followed by UV-Vis spectroscopy. Time dependent absorbance at 530 nm was recorded with reaction time. In the presence of peptide, the rate of reduction of Au ions was dependent on the aqueous conditions, being faster for pH 5.0 and slow for pH 7.5. (b) FTIR analysis. FTIR results indicate that LL37 conjugated to Au NPs have a random coil structure (amine-I band at 1646) as it is usually observed by AMPs in aqueous solution. (c) TEM images and histograms showing the distribution of NP diameters. The average diameters for LL37-Au NPs obtained at pH 5.0 (shown in Supplementary FIG. 2c), 6.0 and 7.5 were 21±8, 35±7 and 12±3 nm (n≥100 NPs), respectively. (d) Percentage of NPs with a circular or elongated morphology obtained at different pH conditions.

FIG. 32: Antimicrobial activity of LL37 and LL37-Au NPs. (a) Scheme showing the general procedure. LL37-Au NPs (10 μg/mL in PBS; either or not autoclaved) or LL37 (5 μg/mL in PBS) were added to LB medium containing E. coli (100.000 cells). After 2 or 4 h of incubation with NPs, aliquots of bacteria were plated on LB agar. (b) Colony forming units (CFU) counted in LB-agar plate. Results were normalised by the control (non-treated bacteria). Results are average±SEM, n=3.

FIG. 33: Stability of NPs suspended in culture medium. (a) Zeta potential of LL37-Au NPs and Au NPs suspended in HEPES or DMEM medium containing 10% (v/v) FBS (HaCaT cell medium). (b) Diameter and counts (Kcps) of LL37-Au NPs and Au NPs in DMEM containing 10% (v/v) FBS. The average diameter of NPs (200 μg/mL) suspended in 2 mL of cell culture media was determined by a dynamic light scattering method (DLS) using a Zeta Plus analyzer (Brookhaven), from six independent measurements. In a and b, results are Average±SEM, n=3. The Au NPs (produced by reduction of citric acid, average diameter of 17.4±1.8 nm) resuspended in water or cell culture media were negatively charged. Au NPs were unstable in cell culture media showing sedimentation at some time points (Supplementary FIG. 6b).

FIG. 34: Cytotoxicity of soluble LL37, Au NPs and LL37-Au NPs. (a, b) Keratinocytes were exposed for 48 h to different concentrations of soluble LL37, Au NPs and LL37-Au NPs, followed by the quantification of viability (PI staining followed by flow cytometry evaluation) (a) or ATP by an ATP kit (b). Results are average±SEM, n=3. (c) Human peripheral blood was in contact with LL37, Au NPs or LL37-Au NPs for 1 h and the percentage of activated platelets measured by the flow cytometric quantification of CD62P (P-selectin) expression. Results are average±SEM, n=3. CD62P is a component of the α-granule membrane of resting platelets that is expressed on the platelet surface membrane only after α-granule secretion, which occurs during platelet activation. Platelets are very sensitive to in vitro activation and aggregation, therefore, the less they are manipulated the better, for this reason the whole peripheral blood (diluted in tyrode buffer) were incubated with the NPs instead of isolating platelet rich plasma. The results show no significant effect of Au NPs or LL37-Au NPs in platelet activation, as the percentage of CD62P+ platelets was similar to control (cells cultured in tissue culture polystyrene). As positive control ADP was used, a platelet activator, which induced the activation of 40% of the platelets. (d) Percentage of keratinocytes expressing ROS. Untreated cells and hydrogen peroxide-treated cells were used as negative and positive controls, respectively. Results are average±SEM, n=3. Cells cultured with Au NPs or LL37-Au NPs show low capacity to generate ROS even after 48 h. Therefore, NPs do not induce an oxidative stress. In contrast, cells exposed to LL37 for 5 h or 48 h generate ROS, mainly for concentrations above 10 μg/mL. (e) Measurement of the membrane potential. Results are average±SEM, n=3. To study the impact of LL37 in the cell membrane the membrane potential of keratinocytes after 24 h of contact with soluble LL37, Au NPs or LL37-Au NPs, were evaluated. It was used a method that allows measurement of relative membrane potential by flow cytometry using a membrane-potential-sensitive charge dye, i.e., 3,3′-dipentyloxacarbocyanine iodide (DioC5(3)). DioC5(3) is a cationic peptide that partitions in the cells in a membrane-potential-dependent fashion. Cells upon hyperpolarization, when the interior becomes more electronegative with respect to the exterior, take up more dye and therefore have high fluorescence. Conversely, cells that have undergone depolarization take up less dye (therefore have low fluorescence). As positive control, it was used gramicidin (10 μM), a non-selective ionophore that causes cell depolarization. The results show that keratinocytes become hyperpolarized after 5 h of contact with LL37. This effect was not observed after 48 h. A depolarisation was observed in keratinocytes exposed to Au NPs for 5 h or 48 h while cells exposed to LL37-Au NPs are depolarised only at 48 h.

FIG. 35: Internalization mechanism of LL37-Au NPs. (a) ICP-MS quantification of NPs (100-400 μg/mL) uptake by keratinocytes after 5 h (a.1) and 48 h (a.2). The results are presented in mass of NPs and not mass of gold. Results are Mean SEM (n=3). (b) Internalization mechanism of LL37-Au NPs and LL37 peptide. (b.1) Schematic representation of the experimental protocol. (b.2-b.5) Uptake of rhodamine-labeled LL37-Au NPs (15 μg/mL) and LL37 (1 μg/mL) in keratinocytes after chemical (b.2 and b.3) or siRNA (b.4 and b.5) inhibition of key regulators of clathrin-mediated endocytosis (CLTC and LDLR), caveolin-mediated endocytosis (CAV1), “GPlanchored protein-enriched early endocytic compartment/clathrin-independent carriers” (GEECCCLIC) pathway (CDC42), macropinocytosis (RAC1 and CTBP1) and scavenger receptors (SCARA3). As controls, keratinocytes were incubated with LL37-Au NPs or LL37 peptide without inhibitors. The results are expressed as Mean±SEM (n=3). *P0.05, **P0.01, ***P0.001 indicates statistical significance relatively to control (cells exposed to LL37-Au NPs or LL37 peptide without any inhibitor). NPs 1-6 h 30 min 1-6 h LL37 Inhibitors Wash, harvest and centrifugation FACS Wash, harvest And centrifugation FACS siRNA 24 h (SCARA3). As controls, keratinocytes were incubated with LL37-Au NPs or LL37 peptide without inhibitors. The results are expressed as Mean±SEM (n=3). *P0.05, **P0.01, ***P0.001 indicates statistical significance relatively to control (cells exposed to LL37-Au NPs or LL37 peptide without any inhibitor).

FIG. 36: Internalization mechanism of NPs. (a) Toxicity of chemical inhibitors used for NPs internalization studies. Keratinocytes were exposed for 6 h to chemical inhibitors of several internalization pathways. The quantification of cell metabolism was assessed by an ATP kit. Results are average±SEM, n=3. (b) Inhibitory activity of dynasore in the internalization of transferrin. Transport of FITC-labelled transferrin (1 μg/mL) known to selectively enter cells via clathrin-mediated endocytosis. Dynasore at concentration of 80 μM inhibits the internalisation of transferrin in keratinocytes. Cells were exposed to culture medium with and without dynasore for 30 min, exposed to FITC-labeled transferrin for 3 min, at 4° C., and finally characterized by FACS. ****P<0.0001 indicates statistical significance between groups.

FIG. 37: Intracellular trafficking of LL37-Au NPs in keratinocytes. Cells were exposed for 30 min or 24 h to fluorescently-labelled LL37-Au NPs (100 μg/mL). At the end of each time, keratinocytes were washed with PBS, fixed and stained for endolysomal compartment markers as early endosome EEA1 and late endosome/lysosome marker protein Rab7 (cell membrane was stained for EGFR). Bars correspond to 15 μm. Confocal microscopy was performed to monitor the intracellular location of the NPs following internalization (a and b). Our results indicate that 30-50% of the NPs were located in EEA1 and Rab7 positive compartments up to 24 h.

FIG. 38: Intracellular trafficking of soluble LL37 in HaCaT cells. Cells were exposed for 30 min, 4 h and 24 h to fluorescently-labeled LL37 (1 μg/mL). At the end of each time, keratinocytes were washed with PBS, fixed and stained for endolysomal compartment markers as early endosome EEA1 and late endosome/lysosome marker protein Rab7 (cell membrane was stained for EGFR). Confocal microscopy was performed to monitor the intracellular location of LL37 following internalization (a and b). The results indicate that ca. 50% of the LL37 was within the endolysosomal compartment (EEA1 and Rab7 positive compartments) up to 24 h. In b, results are Average±SEM, n=3 (3 independent runs; 4 image analyses for each run).

FIG. 39: Expression of EGFR, FPRL1 and P2X7 receptors in keratinocytes. (a) Representative flow cytometry plots for the expression of FPRL1, P2X7 and EGFR. (b) Quantification of receptor expression based in the flow cytometry plots. Results are average±SEM, n=3.

FIG. 40: Downstream signalling of EGFR. (a) Schematic representation of the transactivation mechanism of EGFR. (b) ERK phosphorylation in the presence or absence of P2X antagonist. Keratinocytes were incubated with P2X antagonist PPADS (100 μM) and then activated with LL37 (5 μg/mL) or LL37-Au NPs (200 μg/mL) for 4 min or 8 min, respectively. Following activation, the cells were lysed and protein extracted. The levels of ERK (phosphorylated and total) were determined by an ELISA kit. Results are Results are average±SEM, n=3. *, ** means statistical significance of P<0.05 and P<0.01.

FIG. 41: Co-localization studies between soluble LL37 and LL37-AuNPs with P2X7 in HaCaT cells. Cells were exposed for 30 min to fluorescently-labeled LL37 (1 μg/mL) or LL37-AuNPs (100 μg/mL), after which they were washed with PBS, fixed and stained for P2X7. (A) Representative confocal images showing the co-localization between LL37 or LL37-AuNPs with P2X7. Bars correspond to 15 μm. (b) Quantification of the co-localization. Analyses were performed according to the protocol described in Supplementary Information. Results are Average±SEM, n=3 (3 independent runs; 4 image analyses for each run).

FIG. 42: In vivo distribution of LL37-Au NPs. (a) Au content in different organs of mice at day 2 and 15, as quantified by ICP-MS. Animals were injected with a single dose (1 mg per animal) of LL37-Au NPs. Results are average±SEM, n=5. (b) In vivo evaluation of biochemical parameters after 2 days exposure to LL37 Au NPs. Blood Serum collected at day 2 was analysed for the following biochemical parameters: urea, transaminase-GPT and LDH. This analysis showed no relevant changes compared with untreated animal values demonstrating absence of cytotoxic effect in vivo.

### DETAILED DESCRIPTION

In an embodiment, the present disclosure relates to an antimicrobial peptide conjugated with nanoparticles, in particular the antimicrobial peptide LL37-conjugated gold nanoparticles with antimicrobial activity.

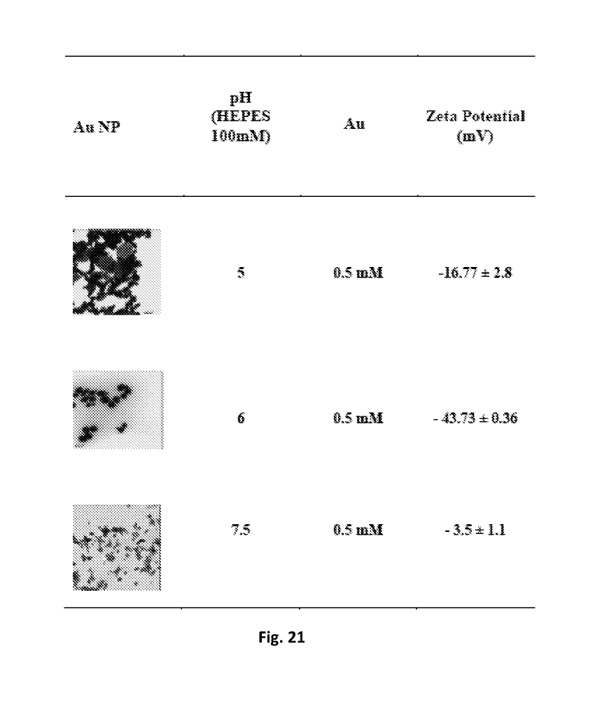

In an embodiment, synthesis and characterization of LL37-Au NPs was performed. Au NPs were synthesized in HEPES buffer (pH 7.5, 5, 6) within 30 min in the absence of LL37 peptide. The Au NPs showed a surface plasmon resonance (SPR) band centered at 720, 650 and 920 nm for pH 7.5, 6 and 5, respectively. Transmission electron microscopy analyses showed the formation of irregular shapes Au NPs for pH 7.5, 6 conditions and of big clusters of heterogeneous and aggregated Au NPs synthesized at pH 5. Zeta potential analyses showed that Au NPs prepared with HEPES at pH 5, 6 and 7.5 had a charge of −16.77±2.8 mV, −43.73±0.36 mV and −3.5±1.1 mV respectively (FIG. 21).

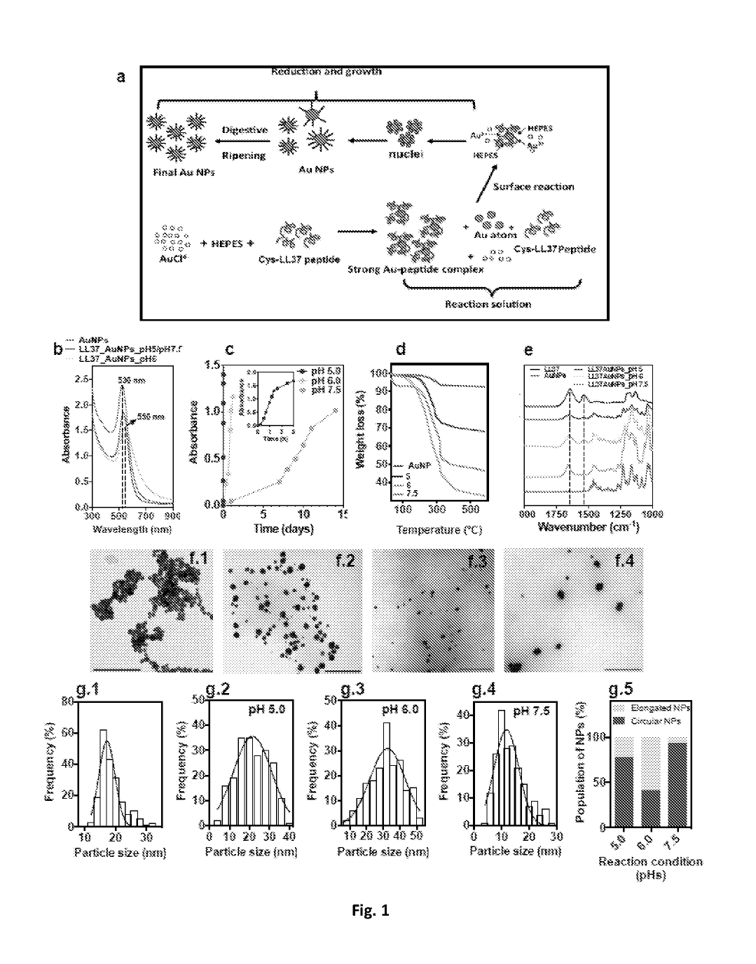

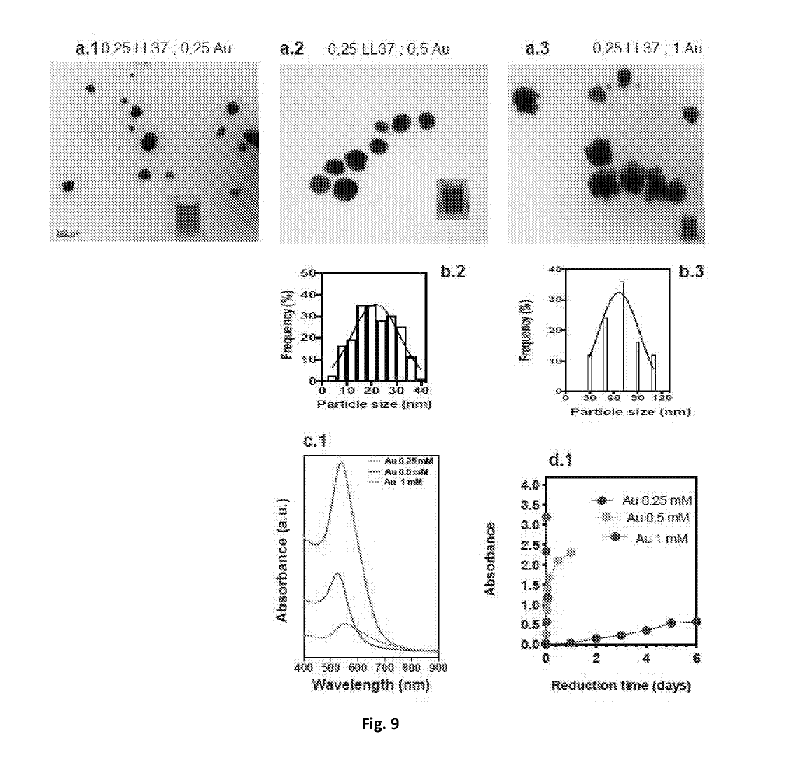

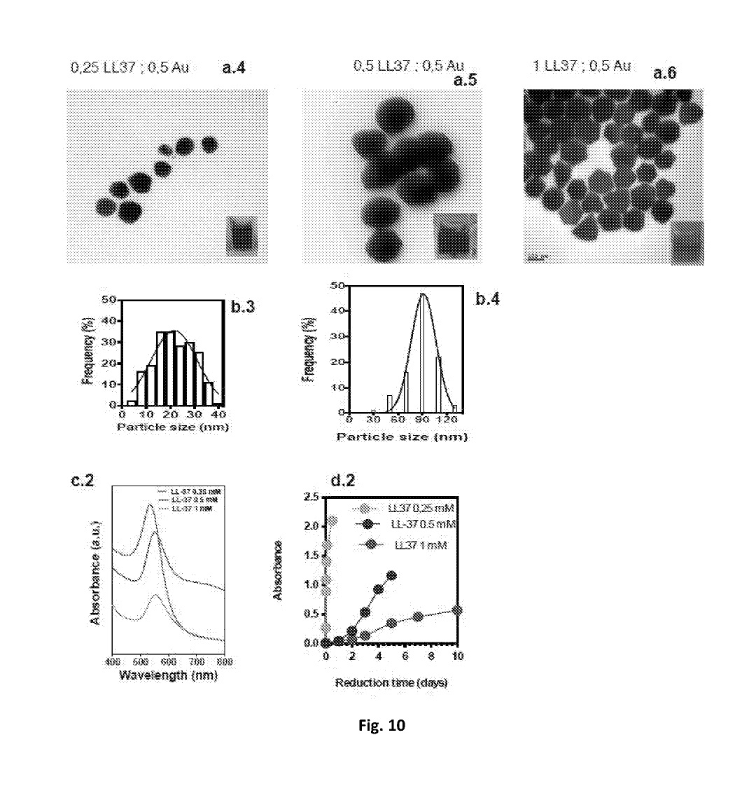

In an embodiment, LL37 peptide modified with cysteine at C-terminus was used to prepare LL37-conjugated Au NPs (FIG. 1a). Several reaction conditions, including initial concentration of LL37 and Au ions (FIG. 9) and pH (FIG. 10) were screened to obtain rapidly Au NPs with low size, relatively low polydispersity and high incorporation of LL37 (FIG. 1a).

TABLE 1

| | pH | Zeta | | | |
|---|---|---|---|---|---|
| | (HEPES | Potential | LL37 % | AU % | Size |
| Name | 100 mM) | (mV) | of mass | of mass | (nm) |
| LL37 AuNP | 5 | 15.4 ± 2.2 | 25 | 46 | 21 ± 8 |
| LL37 AuNP | 6 | 15 ± 1.7 | 50 | 32 | 35 ± 7 |
| LL37 AuNP | 7.5 | 14 ± 3 | 40 | | 12 ± 3 |

In an embodiment, in the absence of HEPES buffer, no reduction of Au ions was observed by LL37 peptide, indicating that HEPES and LL37 peptide act as reducing and capping agents, respectively. In the presence of HEPES buffer, several concentrations of Au ions and LL37 were initially screened. HEPES buffer pH 5 was the buffer selected since at this pH the formation of NPs was faster than at pH 6 or 7.5 (FIG. 10). Therefore, concentrations of Au ions of 0.25 mM and LL37 of 0.5 mM were selected for further screenings since NPs were formed in less than 1 day and showed a relatively low polydispersity.

In an embodiment, the effect of pH in the growth kinetics of LL37-Au NPs was screened. It is known that the reaction pH affects the charge of LL37 peptide and species of gold ions, which in turn influences the interaction of peptide with Au (0), leading to the formation of Au NPs at different synthesis rates. NPs were rapidly formed at pH 5 with saturation being reached in 2 h. In contrast, at pH 6 or pH 7.5, the saturation of NP formation was observed after 1 and 9 days, respectively (FIG. 1c). UV-vis spectra showed the SPR band centered at 530 nm for LL37-Au NPs synthesized at pH 5 and 7.5 while 550 nm at pH 6 (FIG. 1b). According to transmission electron microscopy (TEM) analyses, Au NPs at pH 5, 6 and 7.5 had an average diameter of 21, 35 and 12 nm (FIGS. 1f.2, 1f.3, 1f.4, 1g.2, 1g.3 and 1g.4). Fourier transformed infrared (FTIR) analyses indicated that LL37-Au NPs typically had a random coil structure (amide-I and amide-II bands at 1650 and 1582 cm⁻¹) as did LL37 peptide in aqueous solution (FIG. 1e). Thermogravimetric analyses (TGA) showed that approximately 25% 50% and 40% of the NP mass was peptide for LL37-Au NPs synthesized with pH 5, 6 and 7.5 HEPES, respectively. (FIG. 1d and FIG. 9). Zeta potential analyses showed that Au NPs prepared with HEPES at pH 5, 6 and 7.5). Zeta potential analyses (DLS) showed that the NPs were positively charged (+15.4±2 mV for pH 5 formulation) (FIG. 21).

In an embodiment, HEPES buffer with a pH of 5 was identified, an initial LL37 concentration of 0.25 mM, Au ions of 0.5 mM and one day of reaction as the best conditions to prepare spherical, stable LL37 Au NPs with low size, relatively low polydispersity and to perform further studies.

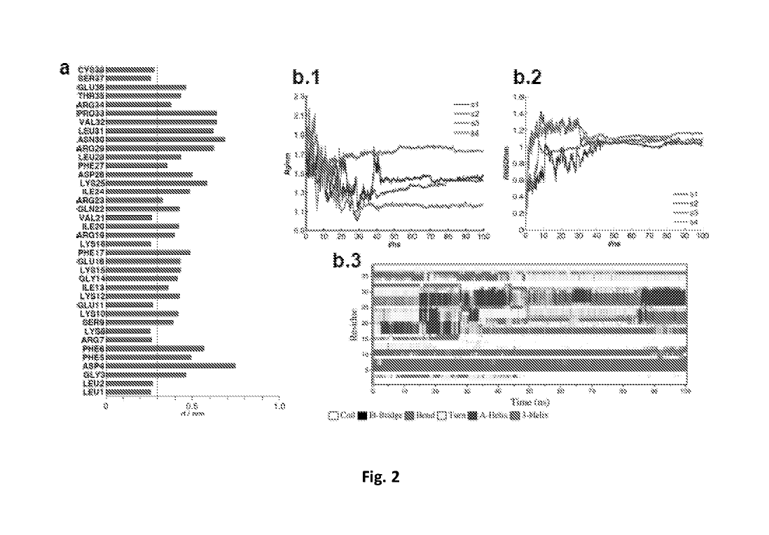

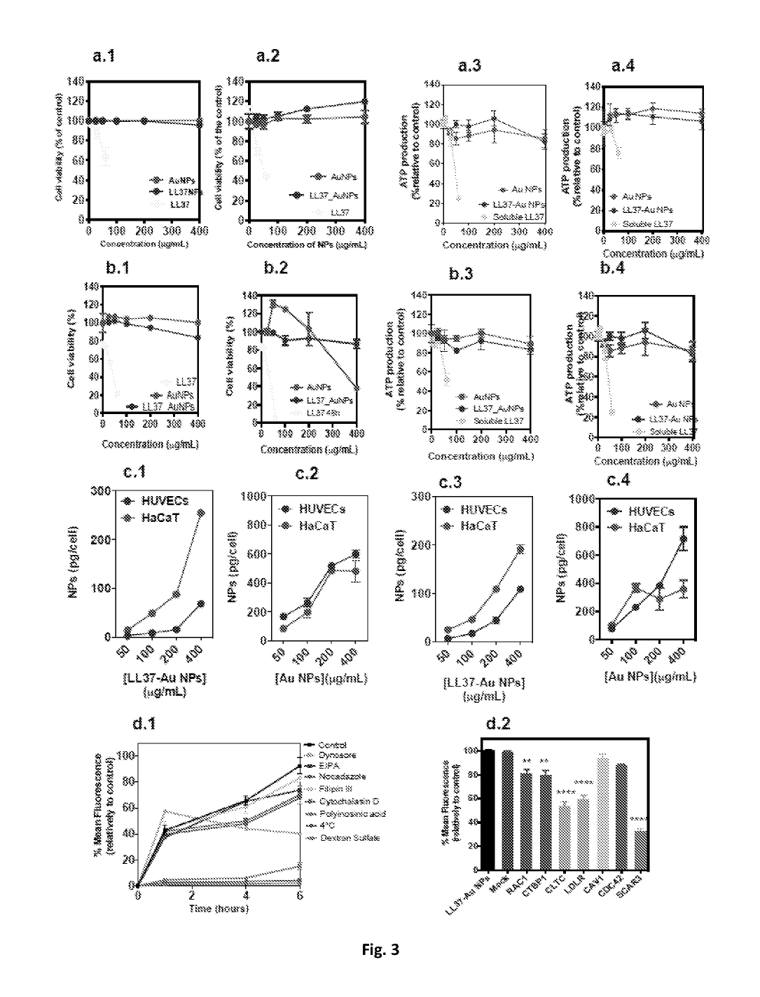

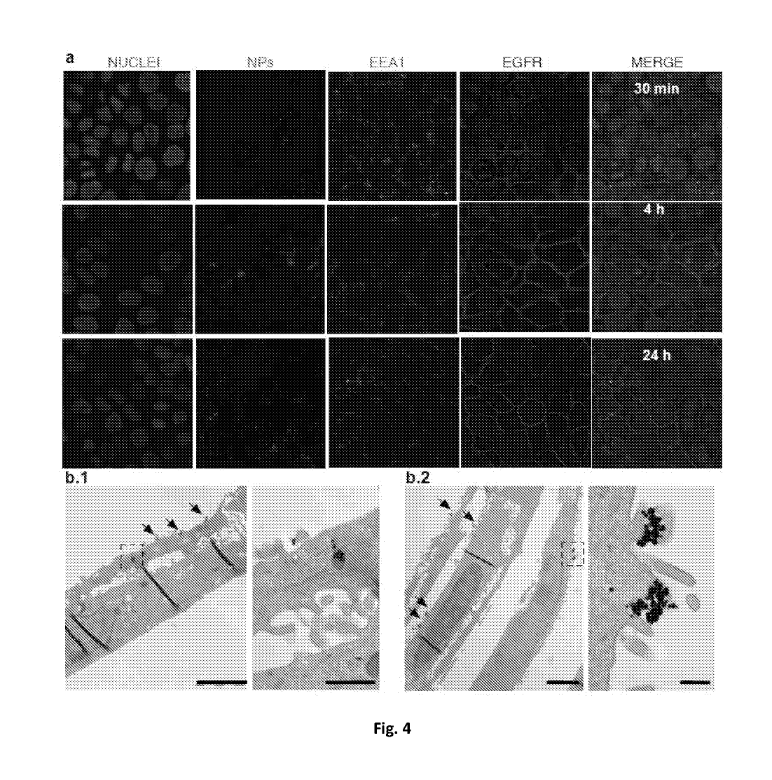

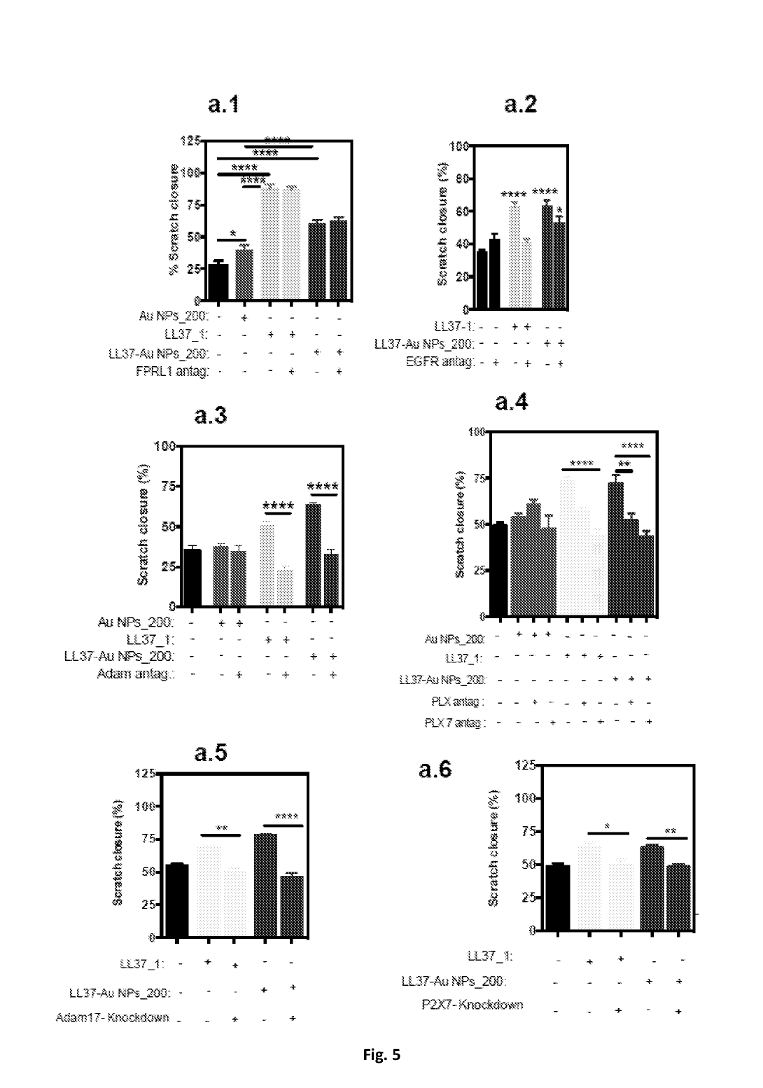

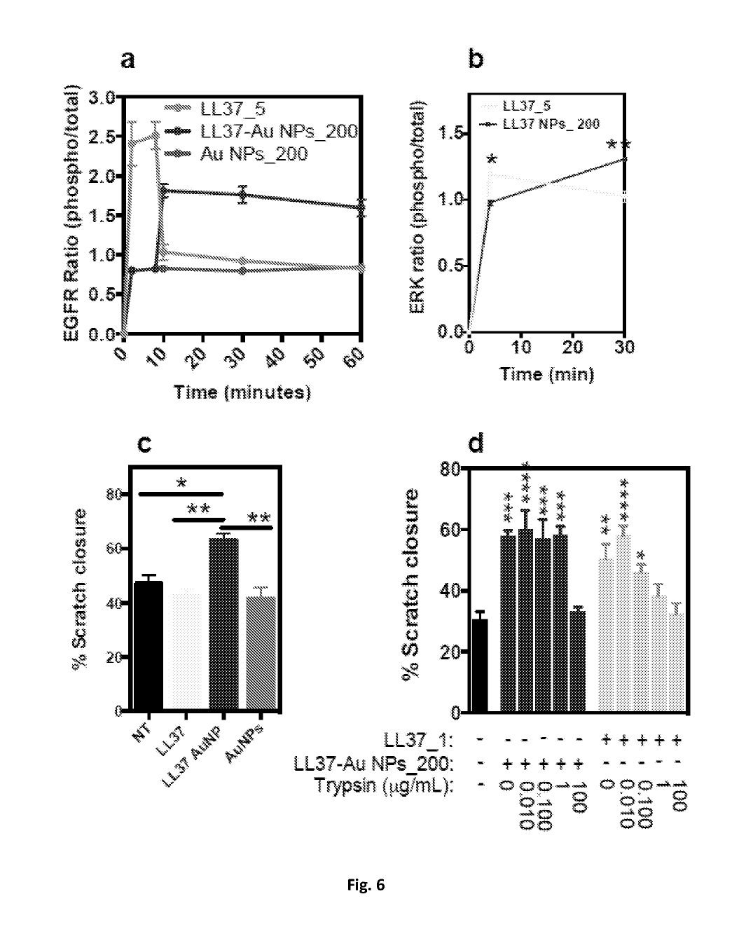

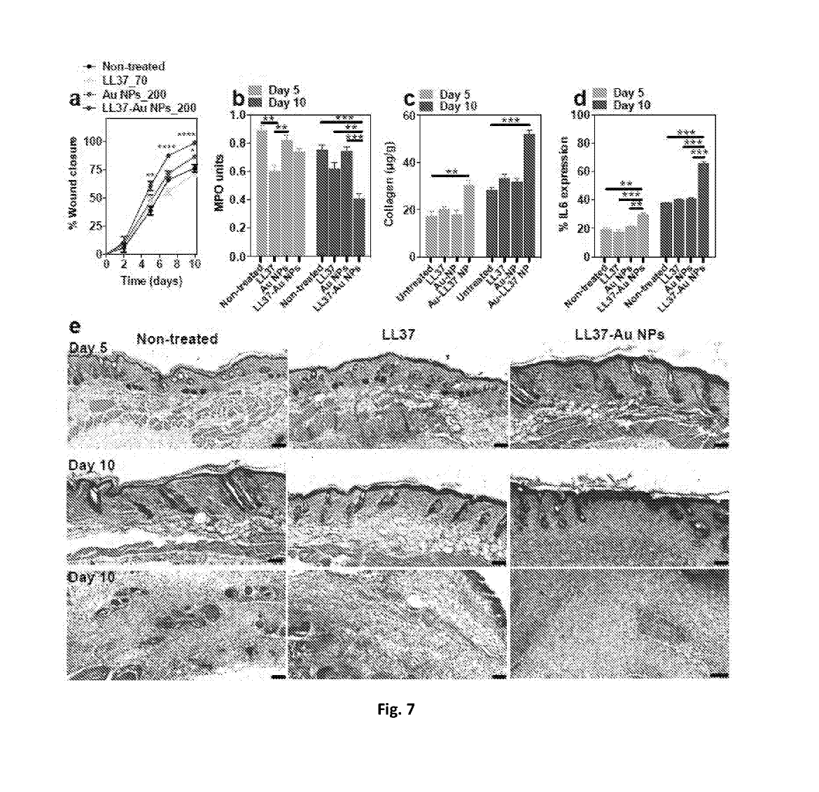

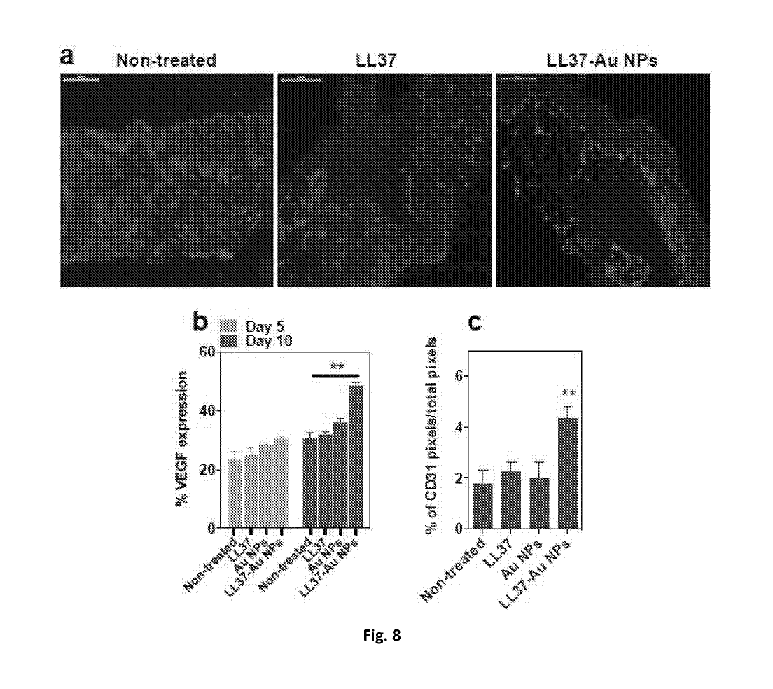

In an embodiment, molecular dynamics (MD) studies. Due to its dimension, 38 amino acids, the diffusion, anchoring and binding processes took longer than smaller peptides previously studied. Overall, most of the residues take 50 ns to achieve a stable positions. However, C-terminal and N-terminal, as well their adjacent residues, seem interact with the AuNP quite strongly. Both terminal moieties have a fast diffusing process, ranging from 25 to 30 ns, and rapidly adopt a stable conformation, as indicated by the constant distance between the amino acid and the AuNP until the end of the simulation (FIG. 2). Moreover, by the end of the simulations those residues present a distance to the AuNP below 0.3 nm (FIG. 2). This indicates that they are in direct contact with the AuNP, which is a requirement for the occurrence of strong biding. The direct contact is characterized by the absence of intermediary water molecules between the residues and the AuNP, which is achieved when the distance between residue and metal is less than 0.30 nm. Nonetheless, a large section of the peptide, from LYS12 to GLU36, take longer times to diffuse and achieve a stable conformation. This may be caused by the fact that those residues do not present as strong attraction to the AuNP when compared to the terminal moieties. However, that section (LYS12 to GLU36) has residues like LYS which is known to have a strong interaction with gold and other metals due to the NH₃⁺ group, meaning that other causes should be postulated. Possibly, after the anchoring of both terminal moieties, the peptide establishes a stable three-dimensional structure (not necessarily a secondary structure) that precludes the subsequent binded of that section. Changes in the complex compactness were evaluated based on the radius of gyration (Rg) which measures the mass of atoms relative to the center of mass of the complex. The obtained results (FIG. 3a) suggest that the peptide adopts a structure more compact after the biding process. This is characterized by the decrease of the Rg throughout the simulations. Despite presenting different final values, the trend is the same. This means that the peptide changes its conformation after the interaction with the AuNP. A similar conclusion was obtained by the analysis of the RMSD of the peptide (FIG. 3b), where a ˜1.0 nm RMSD shift of the peptide was observed when compared to the initial structure. Despite the structural changes of the peptide after the biding, the Rg and RMSD also provide some information on the stability of the AMP-AuNP complex. It is possible to observe a noticeable initial fluctuation of both parameters up to ˜40 ns. This corresponds to the diffusing process and to the initial stages of the anchoring of the peptide to the AuNP. Afterwards, it occurs an abrupt reduction on the fluctuation of the parameters, around 45 ns, resembling a straight line (with some noise inherent to MD simulations) which characterizes the binding stage. Despite some occasional adjustments (see 3 on FIG. 3a around 80 ns), all systems present no substantial change on these parameters during the last 20 ns of the simulations. These results together with the evolution of the distances of each residue (FIG. 1) clearly suggest that a stable complex was achieved. Finally, in order to access if the increase of the concentration of peptide affect the adsorption process, two systems with 6 peptides were tested. Besides simulating the peptides with the AuNP, it was also simulated a system with the peptides in water (no AuNP). This approach was use due to the strong interaction of the peptide with the AuNP, not enabling a proper analysis of the interaction peptide-peptide. Overall, is was not found any evidence of peptide aggregation for both cases. This was found by analyzing the trajectories of the simulations as well the number of hydrogen bounds between the peptides. At the end of the simulation (FIG. 4a) it was possible to observe that the peptides were scattered all over the box which also occurred during all the simulation. To validate this observation, it was tested the number of hydrogens formed with the peptides (FIG. 4b). Despite the small decrease at the beginning of the simulation, the value tends to stabilize around 145 h-bonds (the fluctuation of this parameter can be related with peptide intramolecular h-bonds). The peptide without CYS at the end of the backbone was also investigate, but no differences were found. It was also investigated the secondary structure of each peptide. Despite some few sections that indicate the presence of alpha-helix, there is not a clear secondary structure (FIG. 5) it seems to be not very stable. Finally, it was investigated the interaction between NPs and cell membrane bilayer. From the trajectories of simulation it was possible to access that the complex takes about 20 ns to approach the surface (FIG. 3d) and stabilizes on the bilayer surface during the next 180 ns of simulation. It was also investigated the value of the projected area per lipid (Alip) of the bilayer (FIG. 4). Overall, no distinct variation was found, the observed fluctuation fall within the typical behavior of a lipid surface. In an attempt to extract more information, it was fitted a polynomial of first degree to the data. It was found that the slope was positive for bare LL37 and AuNP/LL37 complex, which means the Alip value tends to increase throughout the simulation. This might be an indicator that the bilayer tends to increase its fluidity end of the simulation. Nonetheless, the slope value, although positive, is very low (in the order of 10⁻⁶) which means that more data is required to support these statements. Regarding the system with the AuNP not functionalized, after analyzing the Alip value it is clear that some severe deformation of the bilayer is occurring. This was confirmed by analyzing the simulation trajectory (FIG. 5). At this stage it was clear that the peptides on the functionalized AuNP are avoid, or delaying, this phenomena of deformation of the bilayer system. It should be stressed that the system with the bare AuNP was simulated 200 ns whereas for the AuNP/LL37 system it was 400 ns. Next, it was accessed the density profiles of the system with the functionalized AuNP (FIG. 6). The presented profiles were averaged for the last 1 ns of the simulation. As conclusions of the analysis, it is possible to state that the AuNP tends to migrate to the bilayer surface (FIG. 6a), characterized by an overlap of the density profile of the AuNP and the bilayer-head. More, some of the peptides are found in the interface water/bilayer, and some are slightly inside the bilayer. After the analysis system without AuNP (FIG. 7), it was clear that at these conditions the LL37-SH tends to remain outside the bilayer. Nonetheless, all of the peptides migrate to the water/bilayer interface and remains there all during all the simulation. This might be the consequence of the combination of two factors. The first one is the possibility of the formation of electrostatic interaction between the positively charged residues of the peptide and the negatively charged phosphate group in the head region of the lipids (this needs to be confirmed!). Second is related with the tendency of residues with hydrophobic moieties having the tendency to migrate to the interior of the bilayer (highly hydrophobic). Next, in order to confirm the previously visualized deformation of the bilayer caused by the bare AuNP, it was extracted the density profiles of that system. As it can be seen in FIG. 8, the AuNP is clearly in the middle of the bilayer.

In an embodiment, the order parameter (SCD) of the lipid alkyl chains was also investigated. SCD provides a direct measure of the flexibility of the alkyl chains which can be related with the fluidity of the bilayer. Higher SCD means stiffer alkyl chains resulting in less fluidity. Analyzing the first carbons on the tails, since are those are the ones nearest the bilayer head groups, it is possible to observe a slight increase of the value on the middle of the simulation (200 ns). By the end of the simulation (400 ns) the SCD values are nearly the same as the ones at the star of the simulation. Due to the low variation of the SCD, it is not possible to understand if the AuNP/LL37 complex is affecting the bilayer fluidity.

In an embodiment, LL37-Au NPs are relatively non-cytotoxic. To evaluate the cytotoxicity of the LL37-Au NPs, endothelial cells (human umbilical vein endothelial cells, HUVECs) and keratinocytes (HaCaT cells) were chosen as representative cell types with which the NPs may interact. The cytotoxicity of NPs can be influenced by their physicochemical properties but also by their stability when resuspend in cell culture media. Therefore, it is of utmost importance to study NP stability in cell culture media. Initially, the zeta potential, size and stability of Au NPs and LL37-Au NPs in cell culture medium (DMEM medium supplemented with 10% FBS for HaCaT cells; EGM-2 containing 2% FBS for HUVECs) were studied by dynamic light scattering (DLS). According to DLS studies, the LL37-Au NPs were positively charged when resuspended in water; however, they became negatively charged after resuspension in cell culture medium likely due to formation of biomolecule corona on the surface of NPs (Supplementary FIG. 4a). The Au NPs (produced by reduction of citric acid, average diameter of 17.4±1.8 nm) resuspended in water or cell culture media were negatively charged. Au NPs were more susceptible to sedimentation in both media than LL37-Au NPs (FIG. 4b). LL37-AuNPs did not sediment but aggregate in both media.

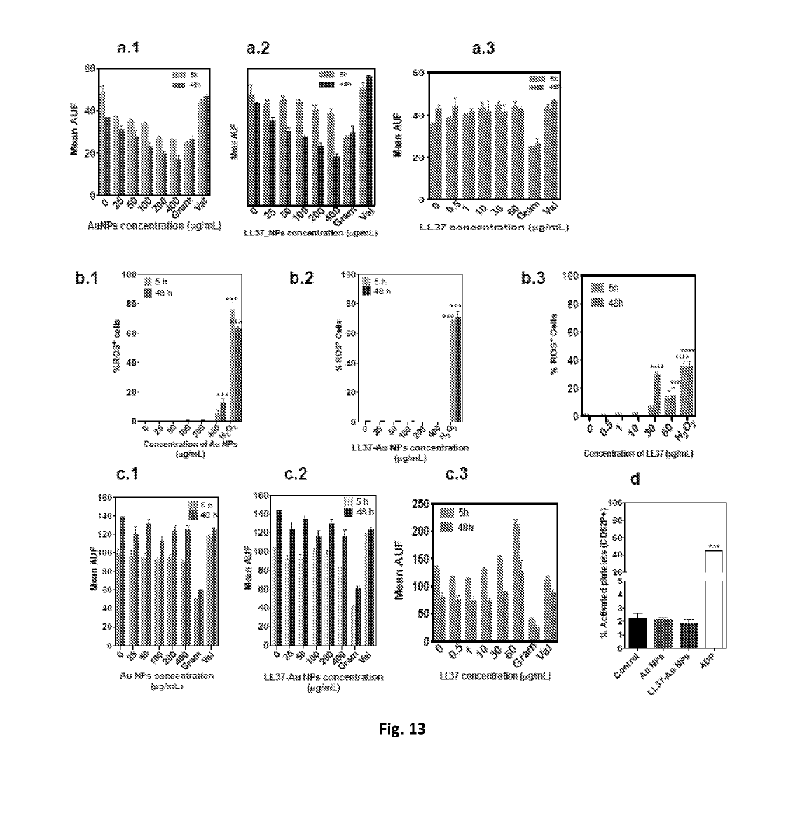

In an embodiment, HUVECs and HaCaT cells were incubated with increasing amounts of soluble LL37, LL37-Au NPs or Au NPs for 5 and 48 h. Cell viability was monitored by PI incorporation (FIG. 3a and FIG. 13b) and ATP production (FIG. 3a and FIG. 13b) while the production of reactive oxygen species (ROS) (FIG. 13c) was evaluated by flow cytometry. There were no significant differences in cell viability, ATP production and ROS generation for HUVECs and HaCaT treated with soluble LL37 and LL37-Au NPs up to a concentration of 30 μg/mL and 400 μg/mL (ca. 100 μg/mL of LL37), respectively. In contrast, significant cell death and ROS production were observed in HUVECs cultured in the presence of Au NPs for 48 h at a concentration of 400 μg/mL and soluble LL37 at concentration of 30 μg/mL. The previous studies were complemented by measuring the impact of soluble LL37, Au NPs and LL37-Au NPs on cell membrane (FIG. 13d). For that purpose, the cell membranes were labelled with 3,3′-dipentyloxacarbocyanine iodide (DiOC₅(3)), a charged lipophilic dye that emits a fluorescent signal proportional to the membrane potential, and exposed the cells to soluble LL37, Au NPs or LL37-Au NPs. Cells upon hyperpolarization (i.e., cell interior becomes more electronegative with respect to the exterior) take up more dye and therefore have higher fluorescence. Conversely, cells that have undergone depolarization take up less dye (therefore have lower fluorescence). As controls, gramicidin (10 μM) was used, a non-selective ionophore that causes cell depolarization, and valinomycin (10 μM), a K⁺ ionophore that causes cell hyperpolarization. HUVECs become hyperpolarized after 5 h exposure to LL37 peptide in solution at concentrations below 30 μg/mL, while depolarized for concentrations above 30 μg/mL. The membrane potential of HaCaT is not altered for soluble LL37. Moreover, the membrane potential of HaCaT and HUVECs is not altered for LL37-Au NPs or Au NPs up to concentrations of 400 μg/mL.

In an embodiment, to evaluate the hemocompatibility of Au NPs and LL37-Au NPs, the induction of platelet activation was tested. Platelet activation was measured by the flow cytometric quantification of CD62P (P-selectin) expression. CD62P is a component of the α-granule membrane of resting platelets that is expressed on the platelet surface membrane only after α-granule secretion, which occurs during platelet activation. Platelets are very sensitive to in vitro activation and aggregation, therefore, the less they are manipulated the better, for this reason the whole peripherical blood (diluted in tyrode buffer) were incubated with the NPs instead of isolating platelet rich plasma. The results show no significant effect of the LL37-Au NPs in platelet activation, as the percentage of CD62P⁺ platelets was similar to TCPS. As positive control it was used ADP, a platelet activator, which induced the activation of 40% of the platelets (FIG. 13). Overall, the results show that LL37-AuNPs are relatively non-cytotoxic for concentrations up to 200 μg/mL in HUVECs and HaCaT. In contrast, HUVECs were more sensitive to soluble LL37 than HaCaT showing cytotoxic effect at a concentration of 10 μg/mL and 30 μg/mL, respectively. Furthermore, LL37-Au NPs have lower cytotoxic and impact on membrane potential than soluble peptide that for the same concentration of LL37 (up to 100 μg/mL). Differences in the cell membrane composition between HUVECs and HaCaT might account for the differences between endothelial and epithelial cells response on LL37 cytotoxicity as supported by the result of membrane potential analysis (FIG. 13d).

In an embodiment, it has been demonstrated that cytotoxicity of Au NPs in cells is mostly influenced by NP size and shape, surface area, functionalization and concentration used. Spherical Au NPs of size ranging from 4 to 100 nm neither induce any cytotoxicity nor impaired the morphology of various cells such as HeLa, human leukemia, human dermal endothelial and mouse macrophage cells up to 100 μg/mL, even though they are being mostly internalized into cells. In contrast, 1.4 nm Au NPs capped with triphenylphosphinemonosulfonate are more cytotoxic than 15 nm Au NPs with similar surface functionality to human carcinoma cells. Positively charged Au NPs functionalized with long carbon chains are generally more toxic to cells at a lower concentration due to combined effect of strong electrostatic interaction with negatively charged cell membrane and hydrophobicity induced toxicity. On the other hand, negatively charged, and short carbon chain functionalized Au NPs have low internalization and exert no toxicity effect on cells.

In an embodiment, surprisingly, the chemical and physical features of the LL37-Au NPs now disclosed such as spherical shape, in particular 21 nm of average diameter, the negative charge when resuspended in cell medium and their functionalization with LL37 facilitate their low cytoxoxicity in comparison with the positively charged free peptide and the not functionalized Au NPs in both HaCaT and HUVEC cells.

In an embodiment, LL37-Au NPs are lower internalized by HUVECs and HaCaT cells than Au NPs. Internalization of LL37-Au NPs and Au NPs by mammalian cells (HUVECs and HaCaT) after 5 and 24 h exposure was estimated using inductively coupled plasma mass spectrometry (ICP-MS) (FIG. 3e-h). HUVEC and HaCaT cells were preferably chosen as endothelial and epithelial cellular model respectively, to mimic the wound cellular. The internalization of the NPs occurred essentially during the first 5 h since no significant increase was observed after 24 h and both cells (HUVECs and HaCaT) internalized higher amount of AUNPs than LL-37 AUNPs (3- and 2-fold higher, respectively). It was also demonstrated that HaCaT cells internalized larger amount of LL-37 AUNPs while HUVECs internalized AUNPs at a higher degree (FIG. 3f, 3h). Taken together, these results show that LL37 AuNPs are less taken up by HUVECs and HACATs. Previous studies have demonstrated nanoparticles may aggregate and change their physical characteristics when exposed to the culture medium or they can sediment quickly, causing the dose of nanoparticles on the cell surface to vary; cellular uptake of nanoparticles depends on the ratio of sedimentation to diffusion velocities.

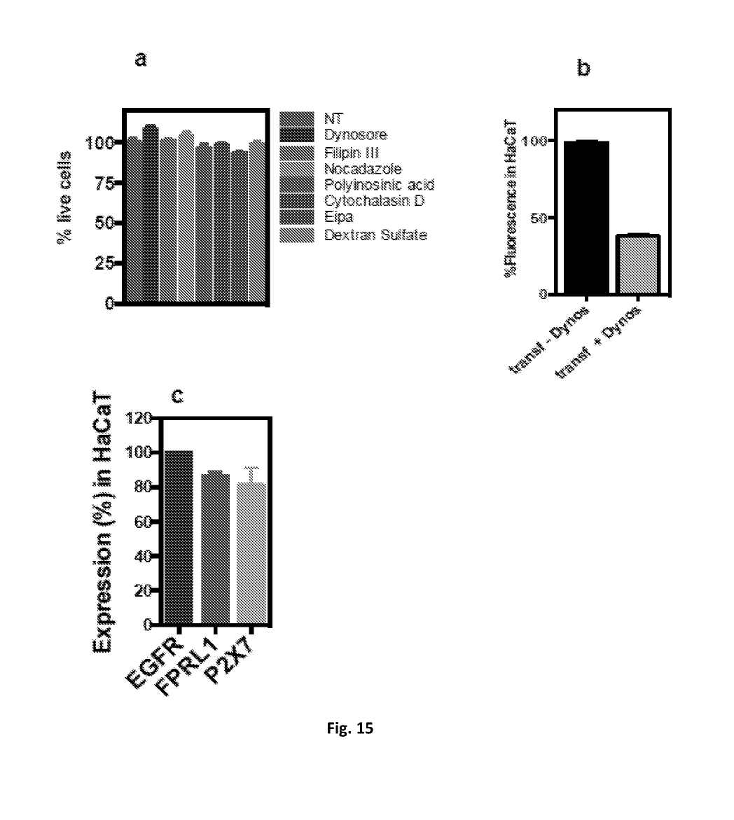

Next, the mechanism of NP internalization in HaCaTs was also studied. Cells were incubated in the presence of endocytosis chemical inhibitors on concentrations that were not cytotoxic for the cells (FIG. 15a), after which, fluorescently labeled NPs (Rhodamine conjugated LL37-AuNPs) were added and the internalization process monitored by flow cytometry. Filipin III inhibits cholesterol dependent internalization mechanisms, nocodazole inhibits microtubule dependent pathways, cytochalasin D inhibits all pathways dependent on actin, dynasore inhibits clathrin-mediated endocytosis polyinosinic acid and dextran sulfate inhibit scavenger receptors and EIPA inhibits macropinocytosis. Whenever possible molecules that enter by a specific internalization pathway were used as positive controls to show the efficacy of inhibitors (FIG. 15). Scavenger receptors mediated with most impact the internalization pathway of LL37 Au NPs, since cells inhibited with polyinosinic acid and dextran sulfate had no nanoparticles internalization. Scavenger receptors are a family of cell surface glycoproteins able to bind modified lipoproteins and polyanionic ligands. Specifically, class-A scavenger receptors (SCARAs) were shown to be involved in the uptake of nucleic acids, double-stranded RNA, and oligonucleotide-functionalized gold nanoparticles. Different SCARA subtypes were detected in endothelial cells, smooth muscle cells, splenic dendritic cells, fibroblasts, and epithelial cells. Here, using siRNA, the effect of knocking down SCARA3 on the uptake of LL37 AuNPs was studied. SCARA 3 has been found expressed in HaCaT cells and it has been shown PF14-SCO nanocomplexes uptake is mediated by SCARA 3 and SCARA 5 in HeLa cells. Cells were transfected with 100 nM of siRNA, notably, knocking down SCARA3 significantly decreased the NPs internalization by 60% compared to values obtained with scrambled siRNA (FIG. 3 c2). This strongly suggests a central role of SCARA in the uptake of LL37 AuNPs. In addition dynosore, potent inhibitor of endocytic pathways known to depend on dynamin, had an important effect on the NPs internalization too. The results showed an inhibition of 50% after 4 h of incubation, further confirmed by siRNA knockdown for CLTC and LDLR (40% comparing with the scramble siRNA). It is demonstrate that the negative charge of LL37 AuNPs in cell medium facilitates the scavenger receptor mediated uptake than others endocytic pathways as already suggested for cell penetrating peptide nanocomplexes and DNA functionalized gold nanoparticles.

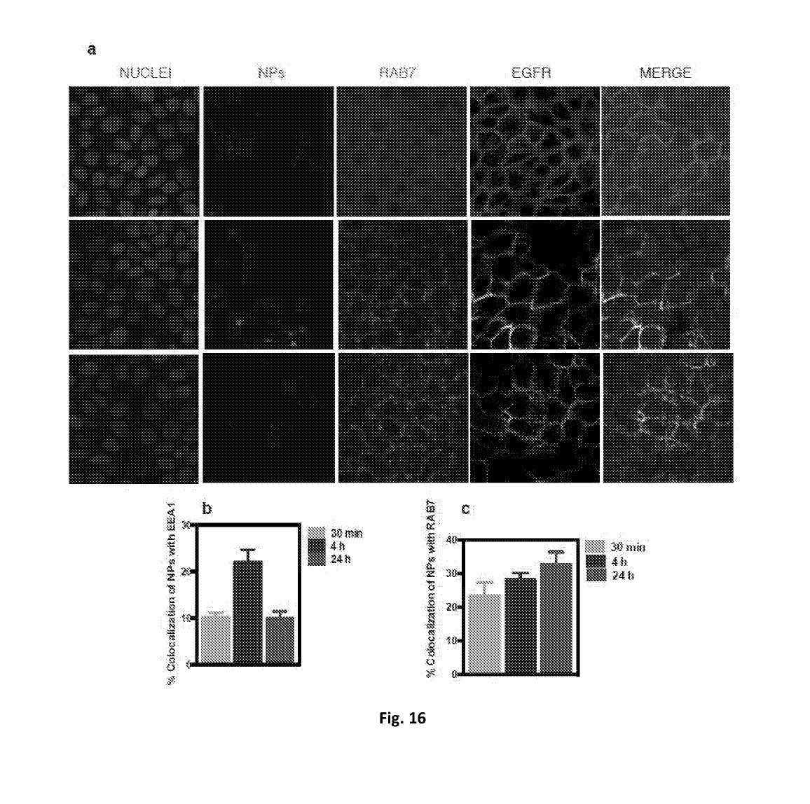

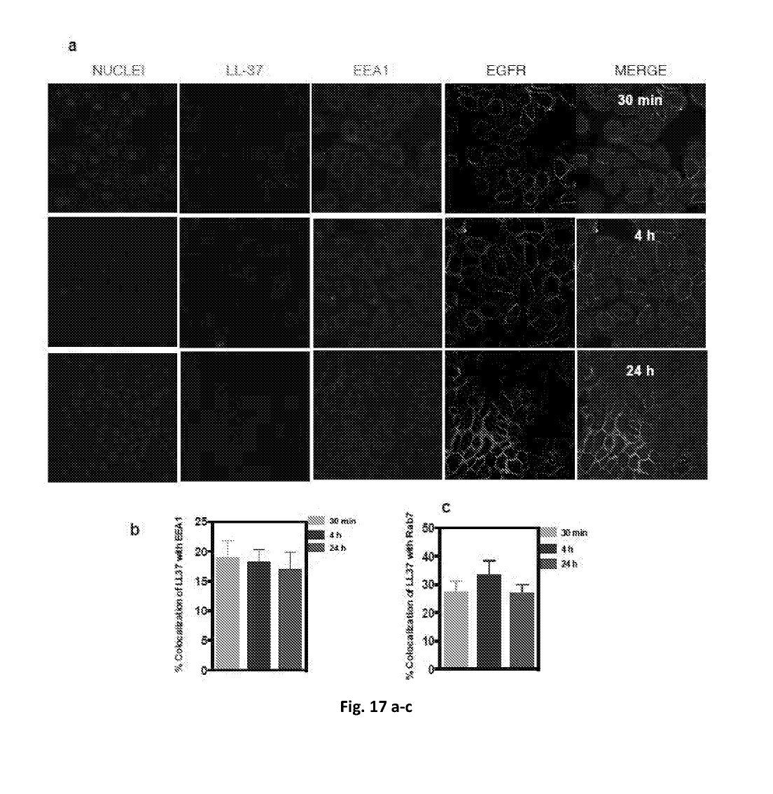

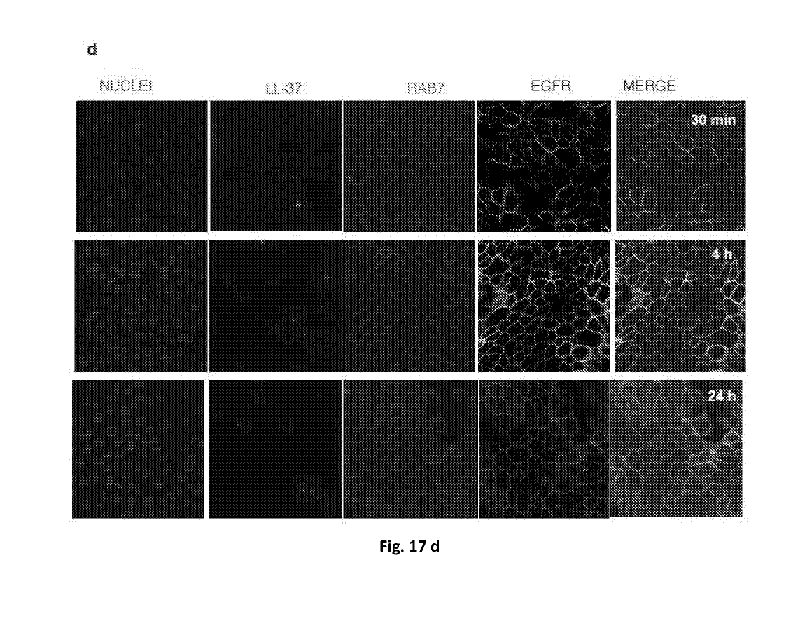

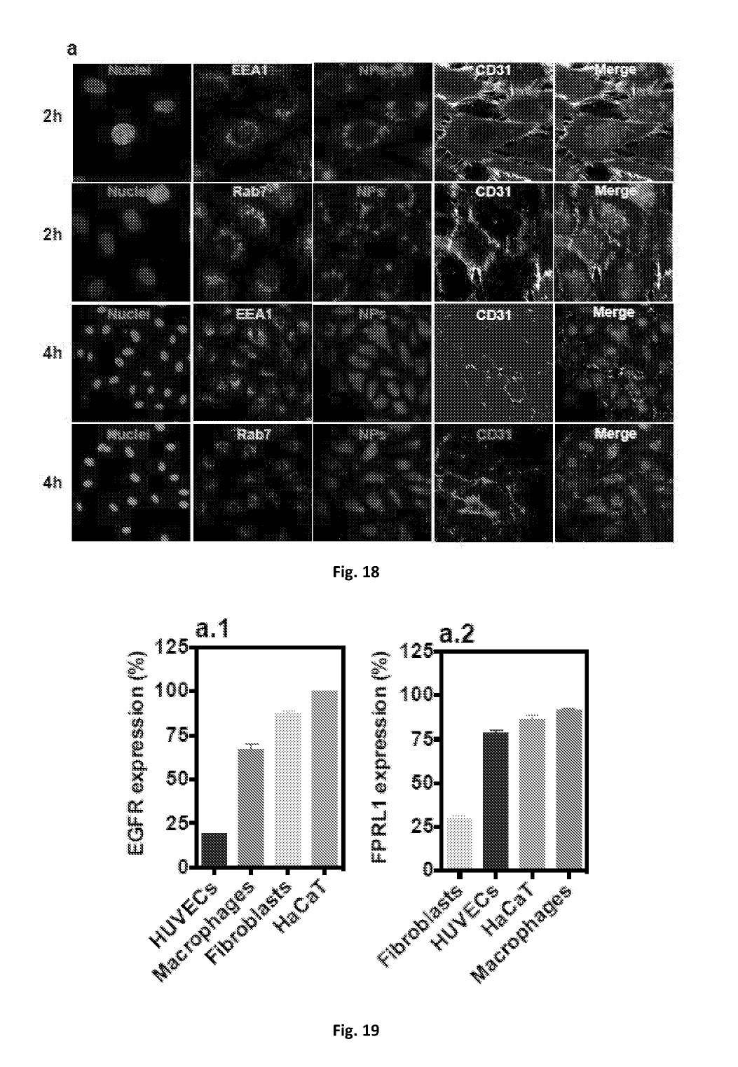

In an embodiment, intracellular accumulation of LL37-Au NPs. Confocal microscopy was performed to confirm that LL37-Au NPs were internalized rather than adsorbing onto the cell surface and to identify the exact intracellular location following internalization. For this purpose, cells were exposed for 30 min, 4 h and 24 h to fluorescently labeled NPs (200 micrograms/mL). Early and late endosomes were stained with EEA1 (FIG. 4) and Rab7 (FIG. 16) respectively and cell membrane stained with anti-human EGFR. Images of cells reconstructed from z-stacks of confocal images indicated extensive cellular uptake of NPs. NPs are mainly observed in the periphery of the cells after 30 min, at 24 h, the NPs were in general localized in the perinuclear region. This agrees with other reports showing perinuclear accumulation of gold nanoparticles in different type of cells. Furthermore, the results did not show a significant percentage of NPs colocalizing neither with early endosome (20% after 4 h) and late endosome (35% after 24 h), as shown by the co-localization of the NPs with EEA1 and RAB7, respectively (FIG. 15b,c). As expected soluble fluorescent LL37 did not colocalize with both EEA1 and RAB7. The cellular uptake of LL37 AuNPs in HaCaT by transmission electron microscopy (TEM) analyses was further characterized (FIG. 4b.1, b.2). TEM results confirm that NPs are taken up by HaCaT and localize within endolysosomal vesicles already after 30 min of NPs incubation. The results also indicate that NPs are taken up by HaCaT with large membrane ruffles engulfing aggregates of NPs when incubation time increases as shown in the FIG. 4b.2. Overall, the results indicate that most of the NPs that are taken up accumulate in endolysosomal vesicles and then the majority of vesicles with clusters of LL37-Au NPs further translocated to the perinuclear region, and after 24 h the nanocomplexes have not escaped endosome yet.

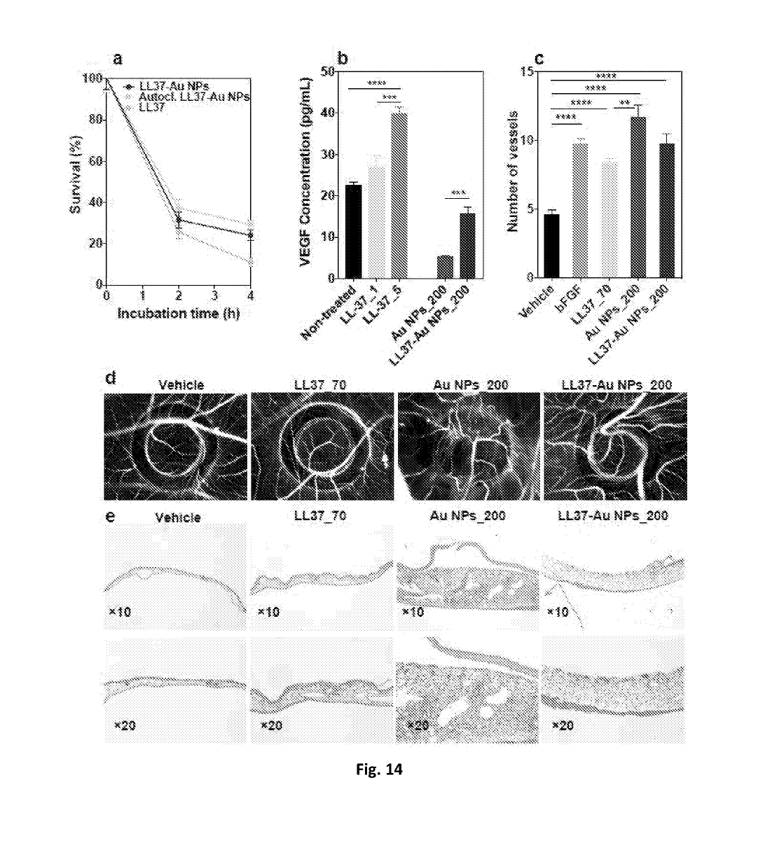

In an embodiment, LL37-Au NPs have antimicrobial and pro-angiogenic properties as soluble LL37. To evaluate whether LL37-Au NPs maintained the bioactivity of LL37, the assessment of their antimicrobial and pro-angiogenic properties was made. The antimicrobial properties of LL37-Au NPs (10 μg/mL) have been assessed against 10⁵ CFU of E. coli, a gram-negative bacteria. Both soluble LL37 and Au NPs (without peptide) have been used as controls. As expected, Au NPs had no antimicrobial activity while LL37 showed a minimal inhibition concentration (MIC) of 5 μg/mL (FIG. 14a). On the other hand, LL37-Au NPs have high antimicrobial activity killing more than 85% of the microorganisms in 4 h. No antimicrobial activity was observed in the supernatant of the LL37-Au NPs, which indicated that the peptide was not leached from the NP surface. Importantly, most of the antimicrobial activity of LL37-Au NPs was kept after a sterilization step by autoclave.

In an embodiment, the evaluation of the pro-angiogenic properties of LL37-Au NPs. Previously, it was shown that LL37 binds to formyl peptide receptor-like 1 (FPRL1), a G protein-coupled, cell receptor found on endothelial cells. The formation of new blood vessels is a prerequisite of tissue repair and wound healing. Therefore, it was evaluated the inductive properties of LL37 and LL37-Au NPs on human umbilical vein endothelial cells (HUVECs) to secrete vascular endothelial growth factor (VEGF₁₆₅) (FIG. 14b). The results showed that both LL37 and LL37-Au NPs induce the secretion of VEGF₁₆₅. Next, the assessment of the pro-angiogenic activity of LL37 and LL37-Au NPs in a chorioallantoic membrane (CAM) assay was made. Both LL37 and LL37-Au NPs induced vessel growth. In both cases, the mesenchymal region of CAM was dilated and a higher number of blood vessels and inflammatory precursor cells were observed than in control (CAM treated with vehicle) (FIGS. 14d and 14e). This reaction was similar to the one observed for basic fibroblast growth factor (bFGF) (data not shown), a pro-angiogenic factor and positive control in the experiment. Interestingly, Au NPs without LL37 are also able to induce a pro-angiogenic response. A quantitative analysis revealed that LL37, Au NPs and LL37-Au NPs induced an increase in erythrocyte-filled blood vessels (FIG. 14c).

In an embodiment, LL37-Au NPs induce prolonged pro-migratory properties in keratinocytes than LL37 peptide. Migration of keratinocytes is an important step in skin wound healing. Therefore, the bioactivity of LL37 and LL37-Au NPs in keratinocytes migration by an in vitro scratch assay were examined. Previous studies have shown that LL37 activates keratinocyte migration by the transactivation of epidermal growth factor receptor (EGFR). This process involves the activation of metalloproteinases (likely ADAM10 and/or ADAM17) which cleave epidermal growth factor anchored to cell membrane into a heparin-binding EGF (HB-EGF), which then binds to EGFR. This leads to the phosphorylation of STAT3, translocation into the nucleus and finally the initiation of the transcription of target genes. In addition, there is the phosphorylation of ERK1/2. Recently, studies with another antimicrobial peptide (mellitin) suggest that the activation of the metalloproteases involves the activation of purinergic receptors, in particular P2X7 receptor (ATP-gated ion channel), although it is unclear which pathway/molecule links plurinergic receptors with the activation of metalloproteases.

In an embodiment, the pro-migratory properties of LL37 peptide, LL37-Au NPs and Au NPs were evaluated by an in vitro scratch assay in HaCaT cells, a human keratinocyte cell line. The results show that soluble LL37 peptide at concentrations of 1 μg/mL induce the migratory properties of HaCaT cells and this effect is mediated by the activation of EGFR and not FPRL-1. Similar results were obtained for LL37-Au NPs (200 μg/mL); however, the inhibitory effect of EGFR antagonist (Erlotinib HCl) was not so evident (FIGS. 6a1, 6a2). Further, it was shown a significant decrease on HaCaT migration for LL37 and LL37-Au NPs when cells where pre-incubated with Marimastat, a general ADAM inhibitor, PPDAS, a broad spectrum purinergic receptor antagonist and A-740003, a specific P2X7 chemical inhibitor, demonstrating their involvement on the EGFR activation mechanism (FIGS. 6a3, 6a4). Further, with the aim to understand which specific ADAMs and to confirm the P2X7 involvement on EGFR transactivation mediated by soluble LL37 and LL37 AuNPs, a scratch assay after siRNA targeting ADAM 17 and P2X7 knockdown was performed. The results have demonstrated that cells silenced with siRNA for ADAM17 and a P2X7 receptor showed a reduced migratory effect for both LL37 and LL37-AuNPs.

In an embodiment, the phosphorylation of EGFR was evaluated. In line with previous results, the phosphorylation of EGFR by LL37 peptide was rapid (peaked at 8 min) and persisted for 10 min. Surprisingly, the phosphorylation of EGFR by LL37-Au NPs peaked at 10 min of contact and persisted for at least 60 min. The phosphorylation level was not significantly affected in the two concentrations tested (15 and 200 μg/mL) (FIG. 7a1). In addition, LL37-Au NP did not show a prolonged phosphorylation of EGFR when the NPs not internalized by the cells were removed from cell surface (data not shown). In a subsequent step, the examination of whether the prolonged phosphorylation of EGFR was correlated with a prolonged phosphorylation of ERK1/2 was conducted. Results have shown the phosphorylation of ERK1/2 by LL37 peptide peaked at 4 min and then decrease reaching a lower value than LL37-Au NPs at 20 min (FIG. 7a2). It was been demonstrated a prolonged activation of ERK1/2 pathway for LL37-Au NPs. Importantly, the phosphorylation of EGFR and ERK1/2 was significantly prolonged in HaCaT cells exposed to LL37-Au NPs than LL37. As result of the prolonged activation of EGFR and ERK pathway by LL37-AuNPs compared with soluble LL37, HaCaT cells treated with LL37 AuNPs in a concentration of 15 μg/mL maintain the capacity to migrate up to 96 h (FIG. 7b).

In an embodiment, the results showed that the activation of P2X7 receptor by both LL37 peptide and LL37-Au NPs leads to the effect of ADAM 17 on transactivation of EGFR as confirmed by pharmacological inhibition experiments and consequently on its phosphorylation. Further, LL37 AuNPs have superior effect on P2X7 induces a prolonged EGFR and ERK pathways activation, hence a prolonged migratory properties of HaCaT cells compared with soluble LL37.

In an embodiment, LL37-Au NPs are higher resistant to proteases than LL37 peptide. To evaluate the stability of LL37 and LL37-Au NPs against proteases, the soluble LL37 and LL37-Au NPs where incubated with several concentration of Trypsin (0,010, 0,100, 1 and 100 μM) for 2 hours at 37° C. LL37 nanoparticles were then centrifuged, resuspended and used to repeat the in vitro scratch assay in HaCaT. Soluble LL37 was incubated with a Trypsin inhibitor for 1 hour before using into cells. In contrast to LL37 peptide, the LL37-Au NPs showed pro-migratory properties in HaCaT cells for high concentrations of non-physiologic concentrations of trypsin (1 μg/mL) (FIG. 4c). The potent bioactivity of LL37-Au NPs was attributed to protection of LL37 by the NPs. According to MD analyses, most of the amino acids of the peptide were at a distance close to or less than 0.30 nm, and thus likely difficult to be reached by the protease catalytic site.

In an embodiment, LL37-Au NPs enhanced in vivo wound healing. To further verify the biological effects of LL37-Au NPs wound healing experiments were performed in a splinted mouse full thickness excisional model. The freshly created wounds were immediately treated with 70 μg of soluble LL-37 peptide, 200 μg of LL37-Au NPs or Au NPs. Wound area was tracked over a period of 14 days and animals were sacrificed on days 5 and 10 for histological and biochemical analysis. After 5^{th} day of the treatment, LL37-Au NPs treated mouse showed an acceleration of wound healing as compared to the control (FIG. 5a). LL37-Au NP-treated mouse showed complete wound healing after 10 days, whereas LL37 peptide-treated mouse showed 50% healing after 10 days. No adverse effects on body weight, general health, or behaviour of the mice were observed after NP treatment. Additionally, the effect of LL37 Au-NPs on wound healing was assessed by histological and immunohistochemical examination of epithelial gap closure. Skin sections were stained with hematoxylin and eosin (H&E) for general observation of skin layers and the extent of collagen deposition in healed tissue was determined by Masson's Trichrome (MT) staining. On day 5, the thick scab and the epithelium layer were formed in wound treated with LL37 Au-NPs compared to LL-37 and Au-NPs treated model. Additionally, wound site was filled with well aligned collagen in case of LL37 Au-NPs compared to other cases with progression of treatment time as shown in MT staining and supported by Sircol collagen results, as well (FIGS. 5c, d). At day 10, a prominent thick epithelium layer was developed in LL-37 AUNPs group and wound re-epithealization and deposition of connective tissue processes were mostly completed, leading to closure of wound (FIGS. 5d, 6a). It has been shown that IL-6 involved in wound healing by regulating leukocyte infiltration, angiogenesis, collagen accumulation and LL-37 mediated epidermal keratinocyte migration. Epidermal keratinocytes have been identified as the main source of IL-6 production in the skin and several host defense peptides including LL-37 have been shown to stimulate IL-6 expression. qRT-PCR analysis indicated that substantial up-regulation in both IL-6 and VEGF at mRNA level occurred after treatment with LL37-Au NPs compared to all the other conditions (FIGS. 5e, f). The quantification of CD31 staining at day 10 also shows an increase of neovascularization in the LL37-Au NPs treated group. The percentage of red pixel represents both endothelial and red blood cells. Significantly, more endothelial and red blood cells were detected in the section of LL37 Au-NPs treated wounds compared with all the other groups (FIGS. 5d, 5g). Neutrophil infiltration in wound was quantified by Myeloperoxidase (MPO) studies. Myeloperoxidase is an enzyme that is found predominantly in the azurophilic granules of neutrophils and can be used as a quantitative index of inflammatory infiltration. MPO activity in wound tissue was significantly decreased after treatment with LL-37 peptide and LL37-Au NPs on day 10, demonstrating anti-inflammatory properties of LL-37 peptide (FIG. 5b). No reduction in MPO activity was found in wound tissue treated with Au-NPs (FIG. 5b). The biodistribution of LL37-Au NPs in different 5 organs of animals showed that there is no accumulation in liver, lungs, spleen and kydney up to 15 days, while Au accumulated in the skin is decreasing from 74% at day 2^{nd} to 10% at day 15^{th} of the in vivo experiment. Further serum biochemical parameters analysis in mice treated with LL37-Au NPs for 2 days showed no relevant changes in the value of urea, transaminase-GPT and LDH compared with untreated animal values demonstrating absence of cytotoxic effect in vivo.

In an embodiment, the results show that LL37-Au NPs enhance wound healing in vivo than soluble peptide and bare Au-NPs, avoiding NPs accumulation in spleen, lung, liver and kidney and without altering their functionality.

Significant efforts have been made in the last twenty years in the synthesis of novel nanomaterials aiming to protect biomolecule from degradation, offer higher stability against enzymes, improve targeting or mechanism of action compared with free molecule, hence prolonging in time in time the biological activity of the drug without cytotoxic effect. Although the wound healing properties of soluble LL37 peptide have been demonstrated, it is unknown the bioactivity of permanently immobilized LL37.

In an embodiment, it is reported a new nanoformulation with LL37-conjugated NPs that have a gold (Au) core and a hydrophilic cationic LL37 peptide shell, made with a quick one-step synthetic process. NPs have controlled size (21 nm) and low polydispersity, which showed antimicrobial activity, higher resistance in the presence of non-physiological concentrations of proteolytic enzymes than soluble LL37, no cytotoxicity against human cells up to 400 μg/ml, prolonged biological properties in keratinocytes in terms of cell migration and activation of P2X7, EGF receptors and ERK pathway. Moreover, LL37 Au-NPs formulation proposed showed huge potential to promote wound healing in vivo without inducing toxic effects.

In an embodiment, it is shown by MD studies a strong and stable encapsulation of LL37 on Au surface that enhance the stability of the formulation in the presence of serum and proteases, and to permit to maintain antimicrobial and angiogenesys activity of the peptide. LL37 Au-NPs are rapidly internalized mostly by scavenger receptor endocytosis and they are non-cytotoxic in endothelial and epithelial cells, at concentrations that are typically cytotoxic for soluble LL37, and accumulate in the cellular endolysomal compartment. Remarkably, the unique interaction of LL37 Au-NPs with membrane structure such as purinergic receptor P2X7 promotes the activation of cellular signalling leading to membrane potential changes and hence to ADAM 17 mediated EGFR phosphorylation and cell migration. The same peculiar NPs-membrane interaction was not found for soluble peptide. The prolonged EGFR and ERK activation leads to a prolonged pro-migratory activity of HaCaT in comparison with LL37. The difference between LL37 conjugated NPs and free LL37 is responsible for the prolonged and enhanced migratory. Furthermore it is shown that mice wounds treated with LL37-Au NPs have a faster wound closure than the ones treated with soluble LL37 without NPs accumulation in the body of mice and adverse effects.

In an embodiment, the present disclosure highlights advantages to use LL37-conjugated NPs either in vitro or in a context of a wound healing model than using soluble LL37. This disclosure shows the differences in terms of stability, cytotoxicity, mechanism of action, and efficacy between soluble peptide and LL37-Au NPs. Although the nanotechnology platform described in this work introduce huge advantages to LL37 to clinical use, further studies are needed before translating this technology for clinical application in human.

Materials. HAuCl₄.3H₂O, Na₃C₆H₅O₇(Sigma-Aldrich) and HEPES (Anal Chem) were used as received. Lyophilized Cathelicidin LL-37 peptide modified with Cysteine (LLGDFFRKSKEKIGKEFK-RIVQRIKDFLRNLVPRTESC-NH₂)—SEQ. ID. 7 was purchased from Caslo Laboratory, Denmark. The peptide was synthesized by conventional solid-phase synthesis, purified by high performance liquid chromatography, and characterized by matrix assisted laser desorption ionization time-of-flight (MALDI-TOF) mass spectroscopy (MS). The purity of the peptides was 96%. Rhodamine B isothiocyanate, HEPES, all the chemical inhibitors for NPs internalization studies, PPADS, A-740003, Marimastat were purchased from Sigma. Formyl Peptide Receptor-Like 1 Antagonist and Erlotinib HCl (EGFR inhibitor) were purchased from Calbiochem and Selleckchem respectively.

In an embodiment, NP preparation. LL-37 (0.5 mM, 1 mg/mL) were dissolved initially in DMF (100 mL) followed by addition of HEPES (950 μL, 100 mM, pH 5). HAuCl₄.3H₂O (10⁻² M, 50 μL) was added to a peptide solution (0.25 mM, 950 mL; therefore the final concentration of HAuCl₄ was 0.5 mM) and the NP synthesis was carried out at 25° C. LL-37 conjugated gold NPs were also synthesized using HAuCl₄.3H₂O (final concentration 0.25 mM, 0.5 mM and 1 mM), and HEPES (100 mM, pH 6 and pH 7.5) by the same procedure at 25° C. The synthesized Au-NPs were centrifuged at 14,000 rpm for 20 min at 4° C. followed by washing with Milli-Q water to remove unreacted peptides and HEPES, frozen and freeze-dried at 223 K using a Snijders Scientific freeze-dryer. Spherical AUNPs were also synthesized via citrate reduction method. In detail, 100-mL sample of deionized aqueous HAuCl₄ (0.5 mM) was boiled in a 250 mL round bottom flask while being stirred, and the (2%) aqueous sodium citrate was added. The reaction was allowed to run until the solution reached a wine red color, indicating the reaction was completed. Fluorescent Au-NPs and LL37 Au-NPs were prepared by addition of DMSO (0.5 mM) solution of TRITC to achieve a final concentration of 25 μM for flow cytometry and confocal microscopy studies. Free TRITC molecules in the colloidal gold solution were removed by centrifugation at 12,000 rpm for 15 min at 4° C. followed by two washings with Milli-Q water. The pellet obtained after centrifugation was redispersed in Milli-Q water.

In an embodiment of the NP characterization, the reduction of HAuCl₄.3H₂O was monitored by UV-vis spectroscopy on a BioTek synergy MX microplate reader. TEM images were obtained using a Jeol JEM-1011 microscope operating at an accelerating voltage of 100 kV. A drop of NPs solution was placed on a 300 mesh copper grid with a carbon-coated formvar membrane and dried overnight before examination by TEM. A minimum of 100 NPs was measured using Image J software for the particle size analysis. FTIR spectroscopy analyses were performed in ATR mode using a JASCO spectrophotometer at 4 cm⁻¹ resolutions with 64 scans. The hydrodynamic diameter and Zeta (ζ) potential of AUNPs and LL-37 AUNPs suspended in water and in cell culture medium (EGM-2 and DMEM) were measured via a Zeta PALS Zeta Potential Analyzer (Brookhaven Instruments Corporation).

In an embodiment of the MD analyses, the MD simulations were performed using GROMACS 4.6.5 code (Van Der Spoel, D.; Lindahl, E.; Hess, B.; Groenhof, G.; Mark, A. E.; Berendsen, H. J., GROMACS: fast, flexible, and free. J Comput Chem 2005, 26 (16), 1701-18). A 6.0 nm diameter Au NP in water was modelled based on the force field parameters described elsewhere. The ff99SB force field was used to model the peptides. In the case of water molecules, the TIP3P model was used. The peptides were first relaxed in aqueous solution 50 ns at 323.15 K and then at 298.15 K for another 50 ns. The resulting conformations were then used as the initial structure for the subsequent simulations. Several independent systems were simulated in view of obtaining data with statistical significance. Hence, a total of 6 peptide positions relative to the Au NP were prepared by rotation of the backbone. All systems were pre-equilibrated using energy minimization simulations. Box volume was relaxed for 100 ps in NPT ensemble under periodic boundary conditions (PBC). The simulations were carried out for 100 ns, with a 2 fs time step, in NVT ensemble under PBC. The temperature was fixed at 298.15 K using the Nose-Hoover thermostat. The electrostatic interactions were treated with the particle mesh Ewald (PME) method and a real space cut-off of 1.0 nm. A cut-off of 1.2 nm was applied to Lennard-Jones interactions. Hydrogen bonds were constrained with the LINCS algorithm.

In an embodiment of the Peptide and Au¹⁹⁷ quantification, The amount of gold in Au-NPs and LL-37 Au-NPs was quantified by ICP-MS, using a Bruckner 820-MS instrument (Fremont, Calif., USA) (confirm with Lugo). The content of LL37 on AU NPs was estimated using Thermogravimetric analyses (TGA). TGA studies were performed using a TA Instruments model Q500 over the temperature range of 30-600° C. at a heating rate of 10° C./min in presence of N₂ gas.

The antimicrobial activity testing may be: Escherichia coli (ATCC 25922) was grown at 37° C. and maintained on LB plates (Luria-Bertani broth with 1.5% agar). LL37 Au-NPs, autoclaved LL-37 Au-NPs and soluble LL-37 (5 μg/mL) were evaluated against 10⁵ bacteria in 1 mL of PBS buffer and incubated for up to 4 h at 37° C. Aliquots (100 μL) were taken out from the respective suspensions at 2 h intervals and diluted in PBS buffer to give 10³ bacteria per mL and plated on LB agar plates followed by incubation at 37° C. Colonies were counted after 24 h of incubation. The collected supernatant was tested for antimicrobial activity as described above.

In an embodiment of the Cell Culture may be a Human Umbilical Vein Endothelial Cells (HUVECs) from Lonza (Walkersville, Md., USA) were cultured in endothelial growth medium (EGM-2, Lonza). Cells under passage six were used in all experiments. HaCaT keratinocyte cell line purchased from CLS service were cultured as recommended by providing company. Briefly, cells were cultivated using DMEM supplemented with 1% (v/v) penicillin and streptomycin (Invitrogen) and 10% (v/v) foetal bovine serum (FBS, Invitrogen) until achieving 90% of confluence. Then HaCaT were first trypsinized and scraped. The cells were sub-cultured at a ratio of 1:3 until achieving the number of cells required for the experiment.

In an embodiment of the cytotoxicity studies in HUVECs and HaCaT may be seeded at a density of 1×10⁴ and 2×10⁴ cells per well respectively and cultured in a 96-well plate for 24 and then incubated with soluble LL37 (0.5, 1, 10, 30, 60 μg/mL), LL37 Au-NPs and AuNPs suspended in culture medium at different concentrations (25, 50, 100 and 200 μg/mL). After 5 and 24 h, CellTiter-Glo® Luminescent Cell Viability Assay (Promega) was used to assess the ATP production of the cells according to the supplier's instructions. For ROS quantification, cells were seeded at a density of 4×10⁴ cells/cm² in 24-well plates and cultured for 24 h. Either LL37-Au NPs or Au NPs suspended in culture medium at different concentrations (25, 50, 100 and 200 μg/mL) were added to cells and left for 5 h and 48 h. Before the treatment ended, cells were dosed with 100 μM of 6-carboxy-2′,7′-dichlorodihydrofluorescein diacetate (DCFH-DA), a dye commonly used to measure intracellular changes in ROS, for 1 hour. After washing the cells twice, they were trypsinized, centrifuged and resuspended in PBS to proceed with flow cytometry analysis (FACS Canto II, BD Biosciences). 1 mM H₂O₂ was used as a positive control for ROS production. In addition, propidium iodide (PI) (1 μg/mL final concentration) was used to quantify cell viability and to exclude the dead cells (i.e. PI positive cells) from the DCFH analysis.

In an embodiment the membrane potential can be determined by: HUVEC and HaCaT cells were seeded at a density of 4×10⁴ cells/cm² and in 24-well plates and cultured for 24 h. Either LL37 Au-NPs or Au-NPs suspended in culture medium at different concentrations (25, 50, 100 and 200 μg/mL, n=3) were added to cells. Soluble LL37 were also used in the concentration of 0.5, 1, 10, 30, 60 μg/mL. After 5 and 48 h, cells were harvested, resuspended in medium with 5 nM of DiOC5(3) (Sigma-Aldrich) and incubated for 5 min at room temperature in the dark, prior to the analysis with the flow cytometer (FACSCalibur, BD Biosciences). Cells treated with gramicidin (10 μM) or valinomycin (10 μM) were used as controls for cell depolarization and hyperpolarization, respectively.

In an embodiment the NP internalization studies can be determined by: HUVECs and HaCaT were seeded at a density of 300,000 cells per well in 6-well plates and cultured for 24 h. Different concentrations of LL-37 AUNPs and bare AUNPs (50, 100, 200, 400 μg/mL) suspended in serum-containing medium were incubated with cells for 5 h or 24 h. After incubation, cells were washed three times with PBS to remove non-internalized NPs. Cells were then detached using 0.2% (v/v) trypsin in PBS, centrifuged, counted and resuspended in nitric acid solution (1 mL, 69%, v/v). After acidic digestion, samples were diluted to 4 mL in Milli-Qwater and gold was quantified by ICP-MS, using a Bruckner 820-MS instrument (Fremont, Calif., USA). Elemental analysis detection of Au¹⁹⁷ was performed after a calibration of the apparatus using gold (Panreac) as standard at 5, 10, 50, 100, 250, 500 and 1000 μg/L. Iridium (Panreac) was used as internal standard at 20 μg/L. Considering the total cell number in each conditions, data analysis permitted to quantify total Au¹⁹⁷ per cell and NPs per cell.

In an embodiment the HaCaT keratinocyte cells were cultured on 24 well plates (1×10⁶ cells/well) and inhibited by one of the following chemicals during 30 min before adding a suspension of Rhodamine-labelled NPs (15 μg/mL): dynasor (100 μM), cytochalasin D (100 μM), nocodazole (25 uM), filipin III (1 μM) and polyinosinic acid (100 μg/mL), 5-(N-ethyl-N-isopropyl)amiloride (EIPA), dextran sulfate. The inhibitor concentrations were based in values reported in literature and further validated by us to have no cytotoxic effect over the period of the assay (6 h), as confirmed by viability assay. The incubation of the cells with NPs for different times was performed in the presence of the inhibitor. As controls, HaCaT keratinocyte cells without NPs and cells incubated with NPs without inhibitor were used. At the end of each time point, cells were centrifuged at 1300 rpm, 20° C. for 5 min, washed 3 times with cold PBS and then resuspended in PBS containing 2.5% FBS (500 μL) for FACS analysis. A total of 10,000 events were obtained per measurement. To validate the inhibitory activity of dynasor it were performed uptake studies of FITC-labeled transferrin, known to selectively enter cells via clathrin-mediated endocytosis. Briefly, HaCaT keratinocyte cells were cultured on 24 well plates (1×10⁶ cells/well) and treated or not with dynasor (80 μM, 30 min pre-incubation), followed by addition of 1 μg/mL FITC-labeled transferrin (Life Technologies). The transferrin was allowed to bind for 3 min at 4° C. Cells were then evaluated as before.

In an embodiment the NP uptake mechanism was also studied on HaCaT keratinocyte cells by silencing specific proteins of clathrin-mediated endocytosis (CLTC and LDLR), caveolin-mediated endocytosis (CAV1), GEEC-CCLIC pathways (CDC42) and macropinocytosis (RAC1 and CTBP1) and scavenger receptor (SCARA3) by siRNA (Thermo Fisher). Transfection was performed in a 24 well plate with 1×10⁵ cells in antibiotic-free complete medium with 100 nM siRNA and 1.5 μL of Lipofectamine RNAiMAX (Life Technologies) transfection reagent for 24 h. After this initial period, the transfection medium was replaced by complete medium and the cells incubated for another 24 h. Then, cells were cultured with Rhodamine-labelled NPs (15 μg/mL) for 4 h. Once the incubations were terminated, the cells were centrifuged at 1300 rpm, 20° C. for 5 min, washed 3 times with cold PBS and then resuspended in PBS containing 2.5% FBS (500 μL) for FACS analysis. Non-transfected cells were used as controls. In all FACS analysis, a total of 10,000 events were recorded per run. All conditions were performed in triplicate.

In an embodiment the cellular Uptake of LL37-Au NPs by confocal microscopy. HaCaT were plated on a μ-Slide 8 Well, ibiTreat (Ibidi, Germany) (30.000/well) and left to adhere overnight before adding Rhodamine LL37-Au NPs (at 100 micrograms/mL). After 30 min, 4 h or 24 h of NPs incubation, cells were washed three times with PBS and were fixed with para-formaldehyde (4% (v/v)) for 10 min, at room temperature, and washed three times with PBS. After blocking with blocking buffer (BB, PBS solution having 1% BSA) for 1 hour, HaCaT were incubated with mouse anti-EGFR antibody (Abcam, dilution 1:1000) for 2 h, washed three times with PBS and permeabilized with 0.3% Triton X-100/PBS, and blocked with BB for 1 h. Cells were incubated with primary antibodies early endosomal marker EEA1 (Cell Signaling C45B10, 1:100 dilution) and the late endosome/lysosome marker protein RAB7 (Cell Signaling D95F2, 1:100 dilution) diluted in BB according to the manufacturer's instructions and incubated for 2 h at room temperature. Secondary antibodies were anti-mouse Alexa fluor 647 and anti-rabbit Alexa fluor 488 (both from Life technologies 1:1000 dilution). Un-bound antibody was removed by washing two times with PBS before staining the nucleus of cells with Hoechst (Life technologies, dilution 1:1000) for 10 min. Confocal images (40 objective) were taken, using the optimal pinhole for better discrimination between foci and assuring no overexposure or bleed-through between channels. Usually images were composed of four channels (blue, green, red, and far-red), where different interactions were analyzed. Images were exported to ImageJ, and colocalization analysis was performed using the automated co-localization tools available named JACOP.

In an embodiment the transmission electron microscopy analyses can be perform by: HaCaT (1×10⁵ cells/cm²) were plated in 56-cm² cell culture Petri dishes until confluency. Then they were exposed to 100 micrograms/mL of LL37-Au NPs for 30 min, 4 h or 24 h. After incubation, cells were washed three times with PBS to remove the NPs not internalized by cells and then they were fixated for 45 min with 2.5% Glutaraldehyde in PB (0.1M pH −7.4). After 45 min of fixation the fixative was removed and more 1.5 mL of fixative were placed in the plate to scrap the cells. Floating cells were then collected in the eppendorf and centrifuged at 3000 rpm to obtain a pellet that was resuspended in PB 0.1 M. Samples were then dehydrated and embedded. Thin sections (mm) of the samples were then analyzed by TEM.

In an embodiment measurement of VEGF amount in HUVECs can be perform by the following method: HUVECs were seeded at a density of 4×10⁴ cells/cm² in 24-well plates and cultured for 24 h in EGM-2 without VEGF. LL-37 (5 μg/mL), LL37 AUNPs and AuNPs (200 μg/mL) suspended in culture medium without VEGF at different concentrations were added to cells. LL-37 (5 μg/mL) and LL-37 AUNPs (200 μg/mL) were also incubated in the presence of 10 μM of FPRL1 Antagonist. After 24 h of incubation, cell media from all the conditions were collected and used to quantify VEGF amount in supernatants. In order to calculate the amount of VEGF in supernatants, Human VEGF ELISA development Kit (PeproTech) was used according to the manufacture's instructions. A calibration curve was used for the ranging between 0-1000 pg/mL VEGF.

Scratch Assay in HaCaT. HaCaT cells (passage 35-40) were seeded at a density of 2×10⁴ cells/well in fibronectin-coated 96-well plate in DMEM supplemented with 1% (v/v) penicillin and streptomycin and 10% (v/v) FBS. After 48 h, cells were firstly starved for 15 h in DMEM with 0.5% FBS and then incubated with LL-37 (1 μg/mL), LL-37 AUNPs and AUNPs (200 μg/mL) for 5 h at 37° C. and 5% CO₂. All the chemical inhibitors for FPRL1, EGFR, ADAM17 and Purinergic receptor were added into cells 1 hour before and for all the incubation time with LL37, LL37-Au NPs or AuNPs. After treatments, cells were washed twice with PBS to remove non-internalized NPs and a scratch was created with a sterile pipet tip. The detached cells were washed twice with PBS and then plates were re-coated with fibronectin (10 μg/mL in starvation medium) for 1 hour at 37° C. At this point (t=0), the first images were taken on In Cell Microscope 2000 (GE Healthcare) (objective 2×). Then cells were washed and maintained in starvation medium up to 72 h. During this time, cell migration was monitored and more images were taken up to 72 h. Scratch areas were quantified using the AxioVision software (Carl Zeiss). Wound areas were normalized by the initial area (n=8 images). On the other hand, to test the prolonged effect of LL37-Au NPs on HaCaT migration, cells were plated in fibronectin-coated 24-well plate (2×10⁵ cells/well), after 48 h cells were starved for 15 h in DMEM with 0.5% FBS and then the scratch was made with a sterile pipet tip. Only after the cells were incubated with LL-37 (1 μg/mL), LL37-Au NPs and AuNPs (15 μg/mL) in starvation medium up to 96 h. During this time, cell migration was monitored and images were taken and scratch area quantified.

In an embodiment the detection of EGFR and ERK1/2 phosphorylation, can be perform by: the activation of EGFR receptor in HaCaT (80% confluency, in 6-well plates) was induced by starving the cells for 24 h in DMEM supplemented with 1% (v/v) penicillin and adding soluble LL37, LL37-Au NPs and Au NPs for a given period of time. Following treatment, the plates were immersed in ice and the medium was discarded. Then the proteins were isolated from the cells . . . . The levels of EGFR (phosphorylated EGFR and total EGFR) were determined by ELISA kit (R&D Systems). The activation of ERK signaling pathways in HaCaT (80% confluency, in one 96-well cell culture clear-bottom black microplate) were obtained starving the cells for 24 h in DMEM supplemented with 1% (v/v) penicillin and then stimulating them with soluble LL37 (5 μg/mL), LL37-Au NPs and Au NPs (200 μg/mL). After treatments the level of ERK were determined using a phosphoprotein and total protein ELISA kit (R&D Systems ref. KCB1018) according to the manufacture's instructions. Data were acquired with a BioTek synergy MX microplate reader.

For measure some parameters In vivo wound healing and tissue collection can be use. 6-7 week old RjHan:NMRI female mice (Janvier, BE) were anesthesized with isoflurane and the dorsal area was shaved using a depilatory cream a day before the surgery. Two 0.5-mm-thick silicone (Grace biolabs, UK) donut-shaped splints (OD=20 mm, ID=10 mm) were fixed on either side of the dorsal midline, approximately 3.5 cm from the ears and positioned with 6-0 nylon sutures (Monosof, USA). Full-thickness excisional wounds were made using an 8 mm round skin biopsy punch (Kai Europe GMBH, DE), centered within each splint. 10 mice were randomly assigned per group and were administered intradermally at several sites around the wound with only vehicle (0.9% w/v NaCl, Mini-Plasco, BE), 70 μg of LL-37, 200 μg of LL-37 AUNPs, and 200 μg of AUNPs in autoclaved water by sterile insulin syringe (BD medical, France) as a dispersion in 30 μl of vehicle. Two wounds were made on each mouse to increase sample size and to avoid cross-contamination both wounds were administered with the same treatment. Thus, n=10 animals (20 wounds) per group. Wounds were covered with transparent sterile adhesive bandage (IV3000, Smith & Nephew, UK) followed by adhesive fabric tape (BSN medical, France) to prevent the chewing of splints by mice. On days 0, 2, 5, 7, 10, 13 and 16 wounds were digitally photographed by Leica IC80 HD camera (Leica, Swiss). Optical zoom was maintained identical throughout the experiments. Wound areas were quantified using the Jmicro Vision software developed by N. Roduit at University of Geneva, Switzerland. Wound sizes are expressed as percentage of the initial respective wound. On day 5 and 10, three animals per group were sacrificed and wounds along with surrounding tissue were collected for further experiments and bisected into two halves. Remaining animals were sacrificed on day 16. The animal studies were approved by the animal care and ethical committee of health science sector, Université Catholique de Louvain.

In an embodiment, histology and immunohistochemistry assay were conducted: the wound halves were immediately fixed with paraformaldehyde (4% in PBS, 0.01 M, pH 7.4) and after 24 h, the samples were transferred to PBS buffer at 4° C. Wound tissue was embedded in paraffin blocks and sequentially sectioned at 5 μm using a MICROM 17M325 microtome (Thermo Fisher Scientific, DE). Skin sections were stained with hematoxylin and eosin (H&E) to assess the predominant stages of healing and with Masson's Trichrome (MT) green staining to study the extent of collagen deposition in healed tissue during the course of wound healing. Images were taken with an AxioCam camera on an Axioplan microscope (Carl Zeiss GmbH, Oberkochen, DE). All histological analyses were performed on at least 3 wounds per group per time point and images presented are representative of all replicates. A semi-quantitative estimation of MT green staining was performed using FRIDA software (developed by Johns Hopkins University, USA) and the staining values were expressed as staining per unit area. The wound halves (n=3) were embedded in Tissue-Tek O.C.T. compound (Sakura Finetek, CA) and frozen. Sections were cut at 10 μm thickness using a cryostat (Leica Microsystems, GE). An antibody directed against the murine endothelial cell surface marker (CD31) was used to determine the extent of endothelial cell growth in the wound sections. After permeabilization (Triton X-100 0.1% (v/v) in PBS) and blocking (5% (w/v) BSA in PBS), the primary antibody (rat anti-CD31 (1:50, BD Biosciences, USA)) was applied for 1 h at 37° C. Secondary antibody (Alexa Fluor 568 goat anti-rat (1:500, Invitrogen)) was used to visualize the antigen. Finally, sections were incubated with DAPI (Invitrogen) (50 ng/ml) for 5 min to visualize the cell nuclei. Images of entire sections were acquired by a MIRAX microscope (Zeiss, USA). Fluorescence of CD31 and red blood cells was quantified on whole sections using a script of FRIDA software (developed by Johns Hopkins University, USA). Results were expressed as percentage of red pixels over the total amount of pixels within the analyzed surface.

In an embodiment collagen (Sircol) Assay may be conducted by: The homogenate of wound tissue was used to measure the total acid-soluble collagen (types I-V) colorimetrically using a Sircol Collagen Assay kit (Newtown Abbey, UK) following the manufacturer's instructions. Briefly, wound tissue sample (n=3) of 10^{th} day, was homogenized in lysis buffer (100 mM potassium phosphate, 0.1% Triton X-100, pH 7.8) and tissue debris was removed by centrifugation at 12,000×g for 10 min (Biofuge 15 R, Heraeus Sepatech, Chandler, Ariz., USA). Sircol dye reagent was added to tissue extracts, stirred for 30 min at room temperature and centrifuged at 12,000×g for 10 min. Absorbance of the bound dye was measured at 560 nm in a spectrophotometer (Spectramax M2e & program SoftMax Pro, Molecular Devices, LLC, USA). The amount of collagen protein in skin samples was adjusted to the amount of total protein using the BCA Protein Assay kit (Pierce, Rockford, Ill., USA). Collagen concentrations were expressed as μg collagen per gram of total protein.

In an embodiment, a MPO assay may be conducted by: the tissue-associated myeloperoxidase (MPO) assay was performed to quantify the degree of inflammatory infiltration in the wounds. Briefly, wound specimens from each group (n=3) on day 5 and day 10 were collected and snap frozen in liquid N₂ at the time of sacrifice and stored for later assessment. For determination of MPO activity, tissue was placed in hexadecyltrimethylammonium bromide (HTAB) buffer (0.5% HTAB in 50 mM potassium phosphate buffer, pH 6) on ice and gently homogenized. The homogenate was centrifuged (Allegra X-15R, Beckman Coulter, CA) at 2,000×g for 10 min and subsequently ultracentrifuged (Biofuge 15 R, Heraeus Sepatech, Chandler, Ariz., USA) at 18,393×g for 20 min at 4° C. The supernatant (7 μL) was added to 96-well plates (Nunc, Roskilde, DK) together with 200 μl of a 50 mM potassium phosphate buffer containing 0.167 mg/mL O-dianisidine (Sigma-Aldrich, DE) and 500 ppm hydrogen peroxide (Merck, DE). Samples were analyzed in triplicate. MPO activity in the supernatant was measured spectrophotometrically at 460 nm for 30 min. The results were expressed as MPO units per gram of tissue, and one unit of MPO activity was defined as the amount that degrades 1 mmol/min of hydrogen peroxide at 25° C.

In an embodiment, RNA isolation and quantitative RT-PCR. Total RNA was isolated from collected wounded tissues using TRIzol® reagent (Ambion, Invitrogen, USA). RNA samples were subjected to DNase I (Promega, USA) treatment to remove genomic DNA contamination in the presence of RNase inhibitor. The quality and quantity of RNA were evaluated by nanospectrophotometer (NanoDrop 2000, Thermo Scientific, DE). 1 μg RNA was reverse transcribed using first standard synthesis system (SuperScript™, Invitrogen, BE) and oligo (dT) primer (Eurogentec, BE) following the supplier protocol. The resulting cDNA was used as template for 30 cycles of semi-quantitative polymerase chain reaction (PCR) in a T100™ thermo cycler, Bio-Rad, BE. Primers of β-actin (internal control), IL-6 and VEGF were used to amplify respective cDNA by PCR. PerlPrimer software was used to design the primers which are mentioned below. The PCR products were subjected to electrophoresis on Syber™ Safe (Invitrogen, BE) stained 1% agarose gel. All samples were analyzed in triplicates. The fluorescence of DNA was measured by ImageJ software and the semi-quantitative fluorescence values were expressed as relative mRNA expression. Primer sequences are shown in Table 2.

TABLE 2

| Oligo name | Sequence of primer (5′→3′) |
|---|---|
| SEQ. ID. 1-IL-6 | TACAATGAGCTGCGTGTGGCCC |
| forward | |
| SEQ. ID. 2-IL-6 | AGGATGGCGTGAGGGAGAGCAT |
| reverse | |
| SEQ. ID. 3-VEGF | TTCGGGAACCAGACCTCTCA |
| forward | |
| SEQ. ID. 4-VEGF | AATGGCGAATCCAGTCCCAC |
| reverse | |
| SEQ. ID. 5-β-actin | CCGGAGAGGAGACTTCACAG |
| forward | |
| SEQ. ID. 6-β-actin | TCCACGATTTCCCAGAGAAC |
| reverse | |

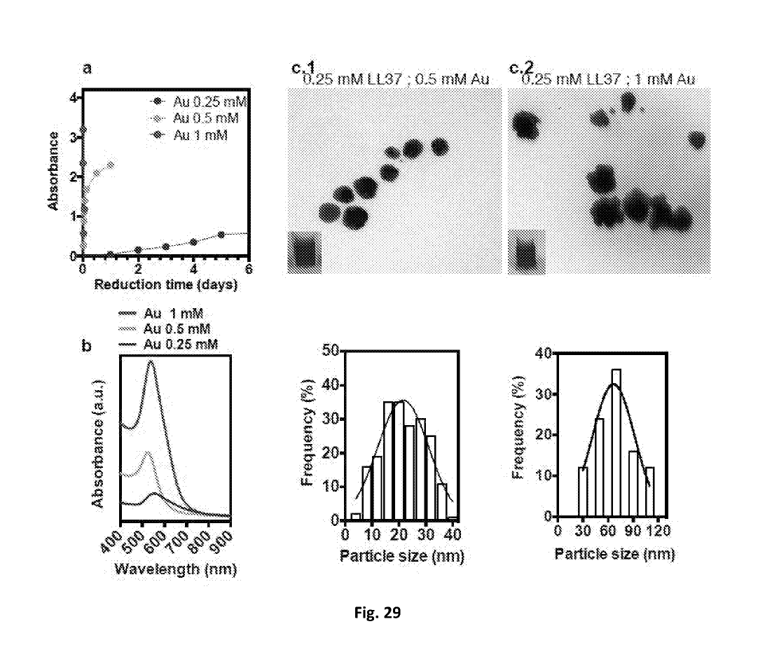

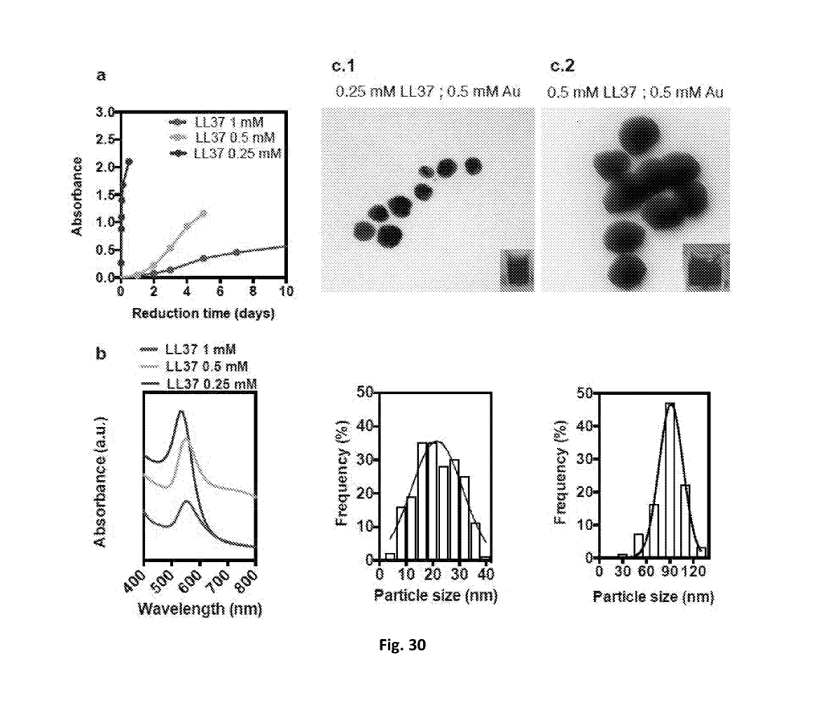

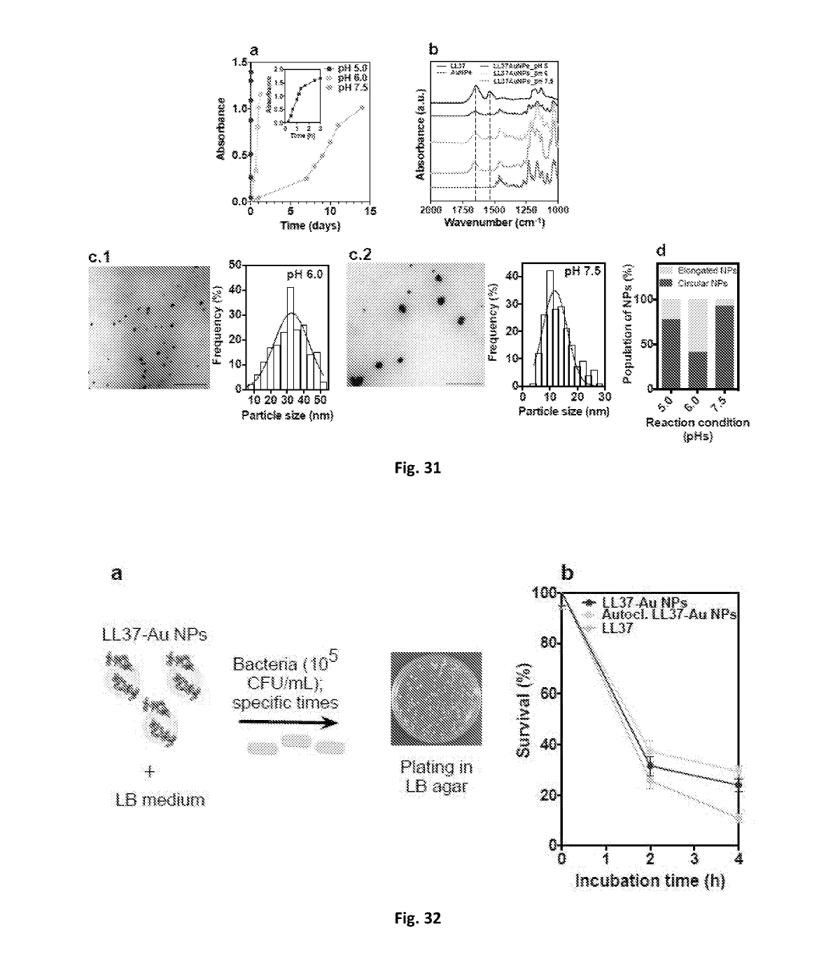

In an embodiment, the synthesis and characterization of LL37-Au NPs was performed. LL37 peptide modified with a cysteine at C-terminus was used to prepare in a single step LL37-conjugated Au NPs (FIG. 22a). Several reaction conditions, including initial concentration of Au ions (FIG. 29), LL37 (FIG. 30) and pH (FIG. 31) were screened to obtain rapidly Au NPs with low size, relatively low polydispersity and high incorporation of LL37 (FIG. 22a). The first benchmarks were to obtain rapid LL37-Au NPs with diameters between 15 and 25 nm (Au NPs with diameters below 10 nm have higher toxicity against mammalian cells), able to maintain their stability in aqueous solution. In the absence of HEPES buffer, no reduction of Au ions was observed by LL37 peptide (data not shown) while in the absence of the LL37 peptide, Au NPs with high polydispersity were synthesized (FIG. 21). Therefore, several concentrations of Au ions and LL37 peptide, were screened, in the presence of HEPES buffer at different pH values (5, 6 and 7.5). HEPES buffer at pH 5 was selected for the initial screening since at this pH the NPs were formed in a few hours (2-3 h) (FIG. 22b) in contrast to pH 6 or 7.5 (see below) which requires between 1 and 9 days, respectively (FIG. 31a). In addition, LL37-Au NPs produced at pH 6.0 were largely non-spherical with large diameter (ca. 35 nm; FIG. 31) while the ones produced at pH 7.5 aggregated overtime (data not shown). Concentrations of Au ions of 0.5 mM and LL37 of 0.25 mM gave rise to NPs up to 1 day, with a relatively low polydispersity and a diameter of approximately 20 nm (FIGS. 29 and 30). Based on these results, it were selected for subsequent tests an Au ion concentration of 0.5 mM and LL37 of 0.25 mM to generate LL37-Au NPs in HEPES buffer at pH 5.0, as a compromise between NP synthesis rate, diameter, stability and polydispersity.

In an embodiment, LL37-Au NPs had a SPR band centred at 530 nm (FIG. 31b), a dominant spherical morphology (FIG. 31) and an average diameter of 21±8 nm (n≥100) (FIG. 29c.1), as determined by transmission electron microscopy (TEM) analyses. Fourier transformed infrared (FTIR) analyses indicated that LL37-Au NPs typically had a random coil structure (amide-I and amide-II bands at 1650 and 1582 cm⁻¹) as did LL37 peptide in aqueous solution (FIG. 22c). High-resolution thermogravimetric and zeta potential analyses showed that approximately 25% of the NP mass was organic (FIG. 22d) and the NPs were positively charged (+15.4±2 mV), respectively. Quantification by spectrophotometry of LL37 peptides that were not conjugated to the Au NPs after reaction indicates that each Au NP had approximately 154 peptides conjugated.

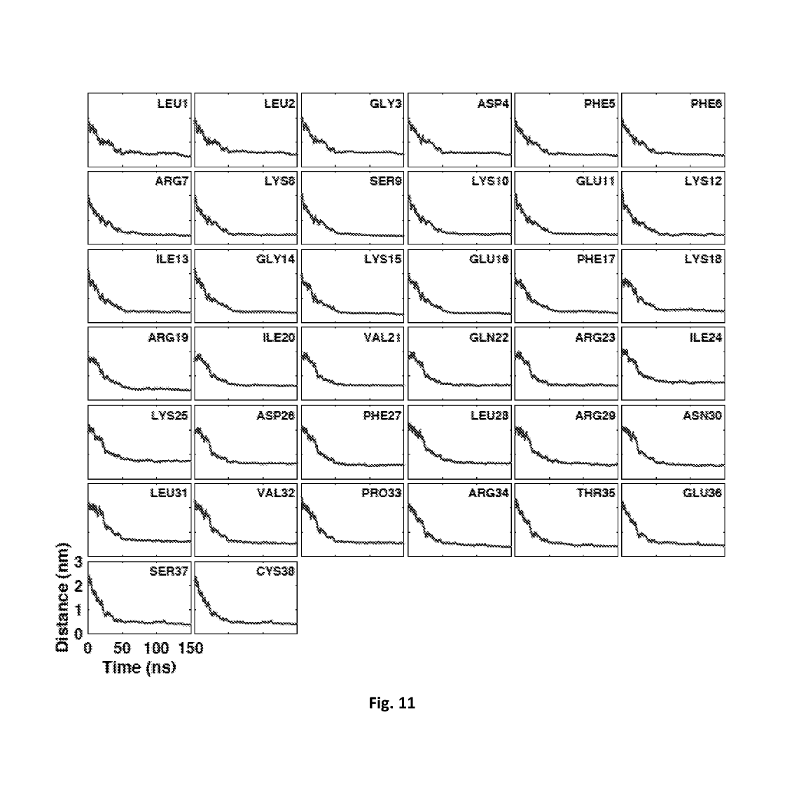

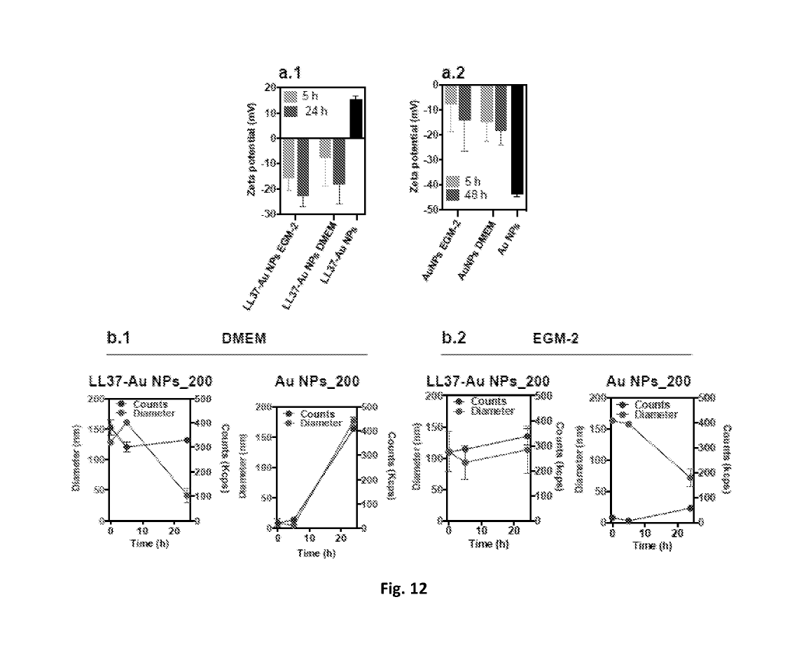

In an embodiment, molecular dynamic (MD) analyses showed that that most of the aminoacid residues took ca. 50 ns to achieve a stable position (FIG. 11). The radius of gyration (Rg) profile suggests that the peptide conformation changes during the interaction with the Au NP, adopting a more compact structure after the immobilization process (FIG. 22f). At the end, some cationic residues of LL37 (ARG7, LYS8, LYS10, LYS12, LYS15, ARG19 and ARG34) as well as CYS38 (the terminal residue of LL37) were in contact (below 0.3 nm) with the surface of the NP while other cationic residues (LYS18, ARG23, LYS25 and ARG29) were distant (FIG. 22e). Although some regions of the peptide (the ones that are distant from the surface of the NP) seem to adopt an alpha-helix structure during the immobilization process, such structure is not present at the end of the simulation, suggesting that no clear secondary structure exists in the immobilized peptide (FIG. 22g).

In an embodiment, to evaluate whether LL37-Au NPs maintained the bioactivity of LL37, it was assessed their antimicrobial properties. The antimicrobial activity of the LL37-Au NPs (10 μg/mL, which corresponds to an immobilized concentration of 2.5 μg/mL of LL37) was evaluated against 10⁵ CFU gram-negative (E. coli) bacteria in PBS buffer at 37° C. (FIG. 32). Au NPs prepared in HEPES buffer without the LL37 peptide were used as a control. As expected, Au NPs had no antimicrobial activity. Surprisingly, LL37-Au NPs or LL37 peptide (5 μg/mL) had high antimicrobial activity killing more than 75% of the microorganisms in 4 h. No antimicrobial activity was observed in the supernatant of the LL37-Au NPs, which indicated that the peptide was not leached from the NP surface (data not shown).

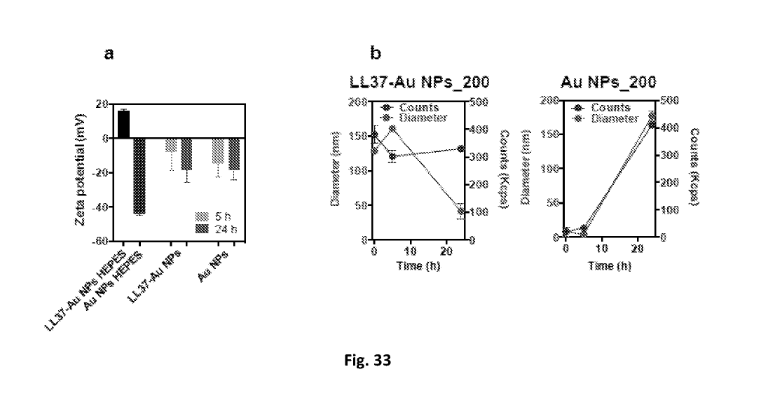

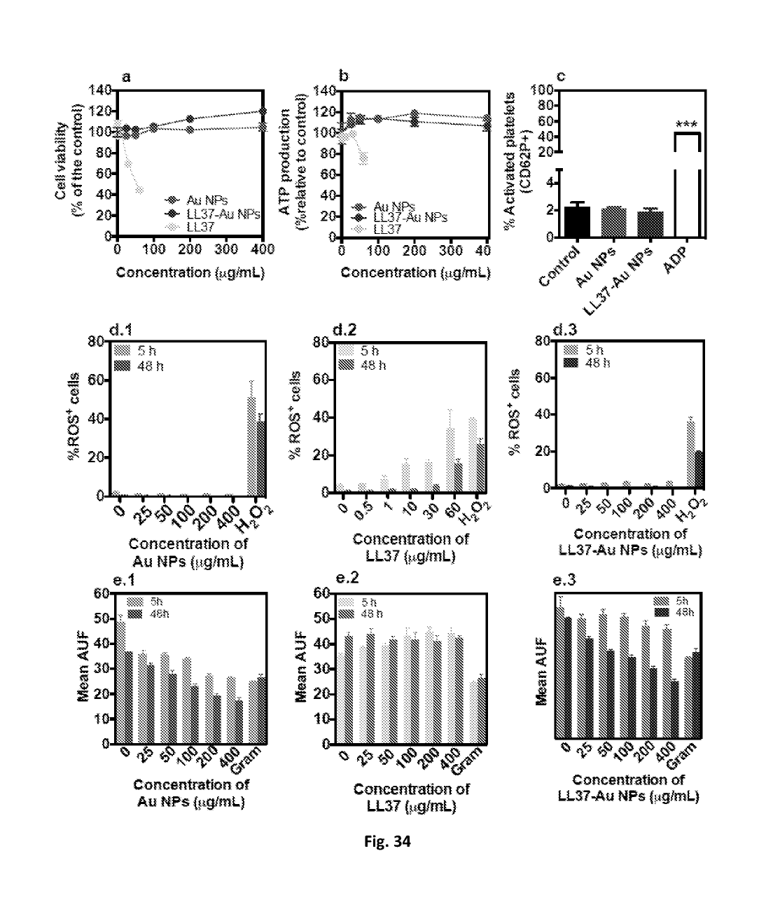

In an embodiment, the interaction of LL37-Au NPs and LL37 with keratinocytes was tested. Human keratinocytes were chosen as a representative cell type with which the LL37-Au NPs may interact with in the skin. LL37-Au NPs were positively charged when resuspended in water; however, they became negatively charged after resuspension in keratinocyte culture medium (DMEM medium supplemented with 10% FBS) likely due to formation of biomolecule corona on the surface of NPs (FIG. 33a). LL37-Au NPs did not sediment overtime but show some aggregation in cell culture media. To assess the biological effect of the NPs, cell viability was monitored by PI incorporation (FIG. 34a), cell metabolism by ATP production (FIG. 34b), cellular oxidative stress by the production of reactive oxygen species (ROS) (FIG. 34d) and cell membrane impact by cell depolarization studies (FIG. 34e). LL37 and Au NPs have been used as controls. Surprisingly, keratinocytes treated with LL37 or LL37-Au NPs up to a concentration of 10 μg/mL and 400 μg/m L (ca. 100 μg/mL of LL37), respectively, have no significant decrease in cell viability or increase in ROS production. Yet, a decrease in cell viability and an increase in ROS production were observed for concentrations of LL37 equal or above 10 μg/mL. Keratinocytes became slightly hyperpolarized after 5 h exposure to LL37 peptide in solution at concentrations above 25 μg/mL, while the effect was lost at 48 h. Interestingly, the membrane potential of keratinocytes was depolarized after exposure to LL37-Au NPs or Au NPs for 48 h. These results suggest a likely interaction of NPs with cell membrane.

In an embodiment and because one aim of the present disclosure is to use LL37-Au NPs to treat chronic wounds it was measured also its hemocompatibility. The LL37-Au NPs were determined to be relatively hemocompatible because they did not activate platelets aggregation (FIG. 34c).

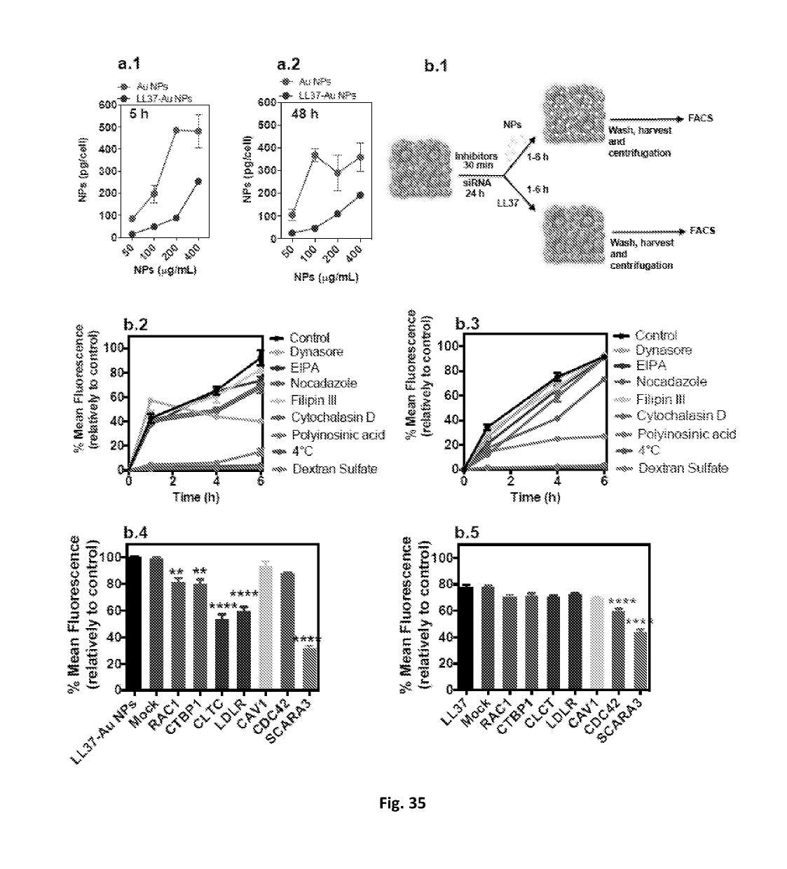

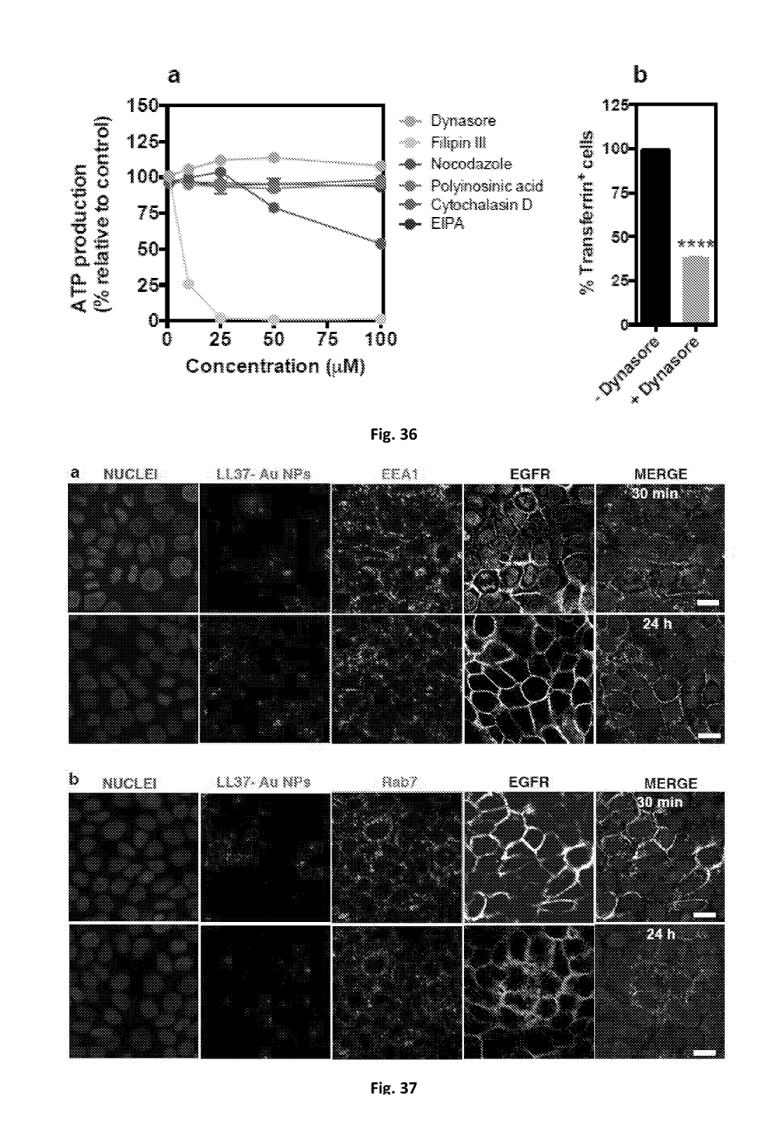

In an embodiment, the bioactivity of LL37 requires its cellular internalization. Therefore it was evaluated the internalization of LL37-Au NPs by inductively coupled plasma mass spectrometry (ICP-MS) (the internalization of LL37 peptide was evaluated below by confocal microscopy and flow cytometry). Human keratinocytes were able to uptake LL37-Au NPs (between 20 and 250 μg/cell depending in the initial concentration of NPs) although less effectively than Au NPs (having similar size; 2 to 7-fold lower, depending on the initial concentration and time of contact) as evaluated by ICP-MS (FIG. 35a). The internalization of the NPs occurred essentially during the first 5 h since no significant increase was observed after 48 h of NP contact with the cells. To identify the mechanism of internalization of LL37 peptide and LL37-Au NPs, keratinocytes were incubated in the presence of endocytosis chemical inhibitors at concentrations that were not cytotoxic for the cells (FIG. 36a), after which, rhodamine-labeled LL37-Au NPs or rhodamine-labeled LL37 were added and the internalization process monitored by flow cytometry (FIG. 35b.1). Various inhibitors such as filipin III, nocodazole, cytochalasin D, dynasore, polyinosinic acid/dextran sulfate and EIPA were used to inhibit cholesterol-dependent internalization, microtubule-dependent pathways, actin-dependent pathways, clathrin-mediated endocytosis, scavenger receptors and macropinocytosis pathways respectively. Whenever possible molecules that enter by a specific internalization pathway were used as positive controls to show the efficacy of the inhibitors (FIG. 36). The internalization of LL37-Au NPs and LL37 was mediated by endocytosis since significant inhibition was observed at 4° C. The results further show that LL37-Au NPs and LL37 were internalized mainly by scavenger receptors, since keratinocytes inhibited with polyinosinic acid or dextran sulfate had no significant NPs or LL37 peptide internalization (FIGS. 35b.2 and 35b.3). To confirm the endocytosis mechanisms involved in NP internalization, keratinocytes were transfected with siRNAs to down-regulate key components of different endocytic mechanisms (FIGS. 35b.4 and 35b.5). It was observed a ˜70% and 60% reduction on LL37-Au NPs and LL37 uptake, respectively, upon downregulation of class-A scavenger receptor 3 (SCARA3), confirming a role of scavenger receptors in NP internalization.

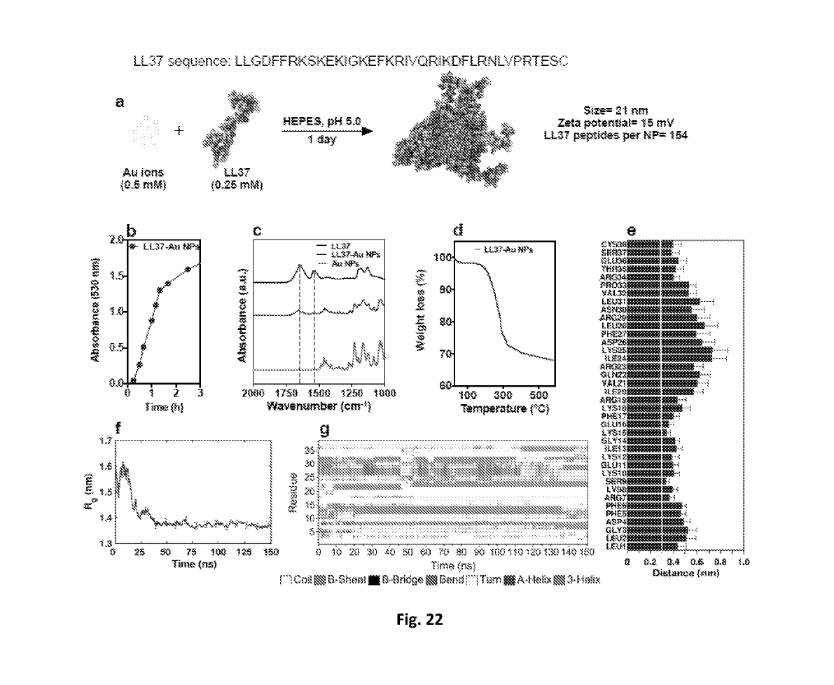

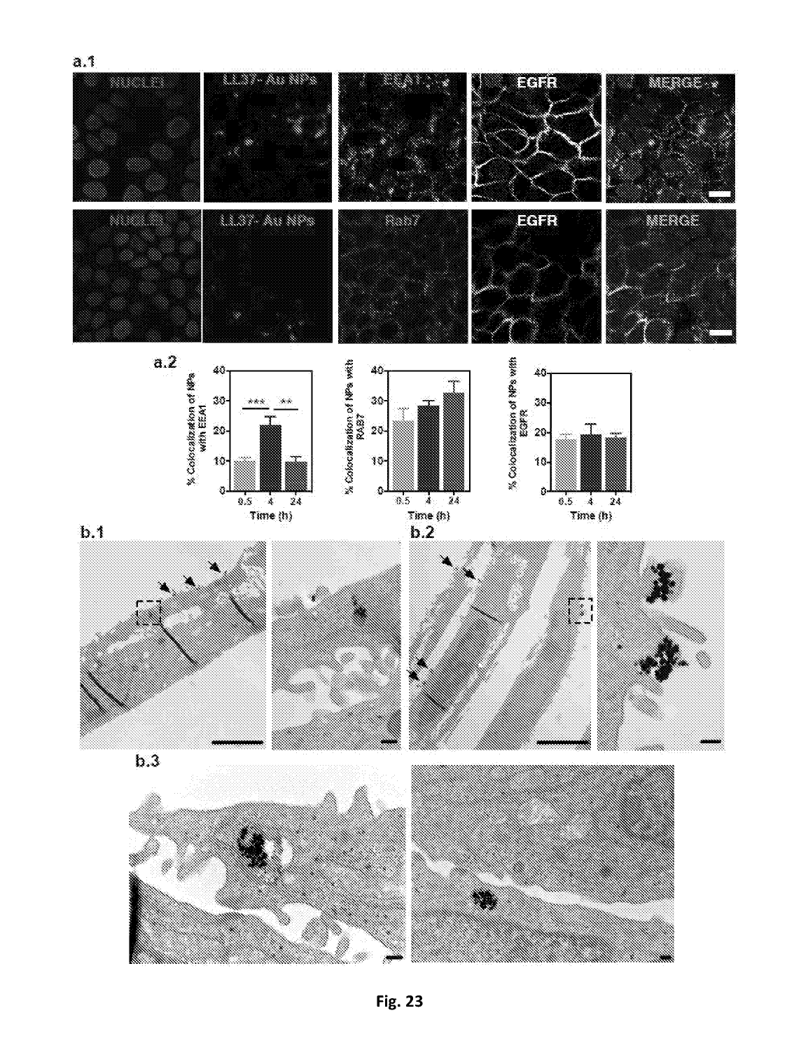

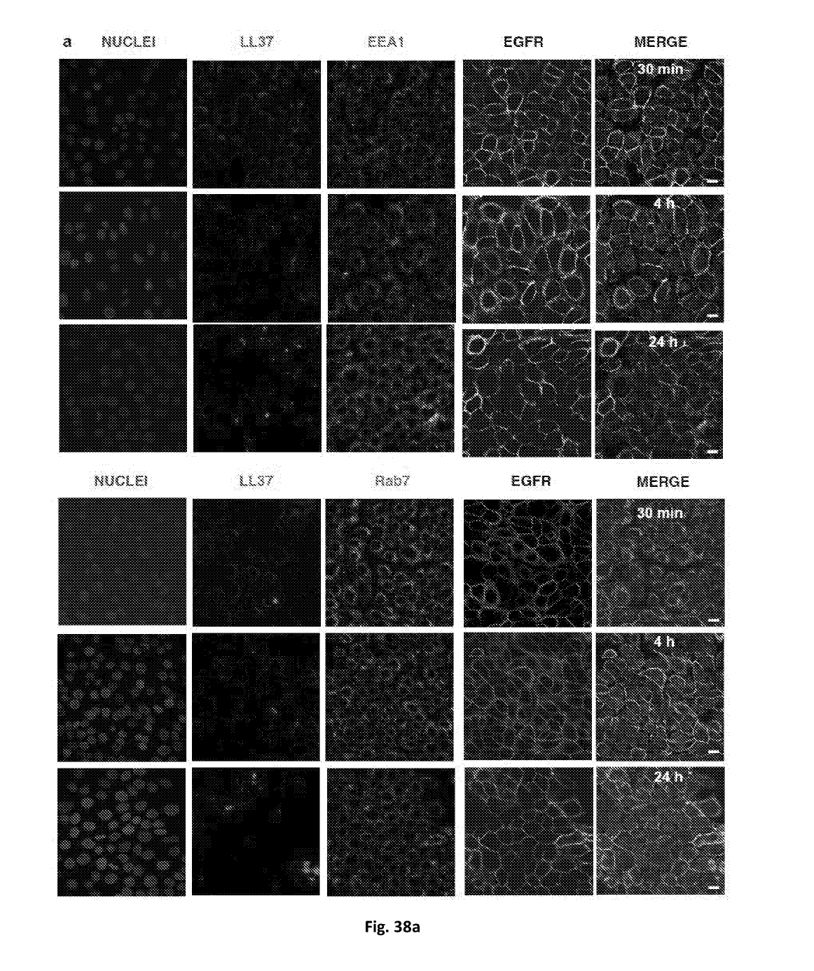

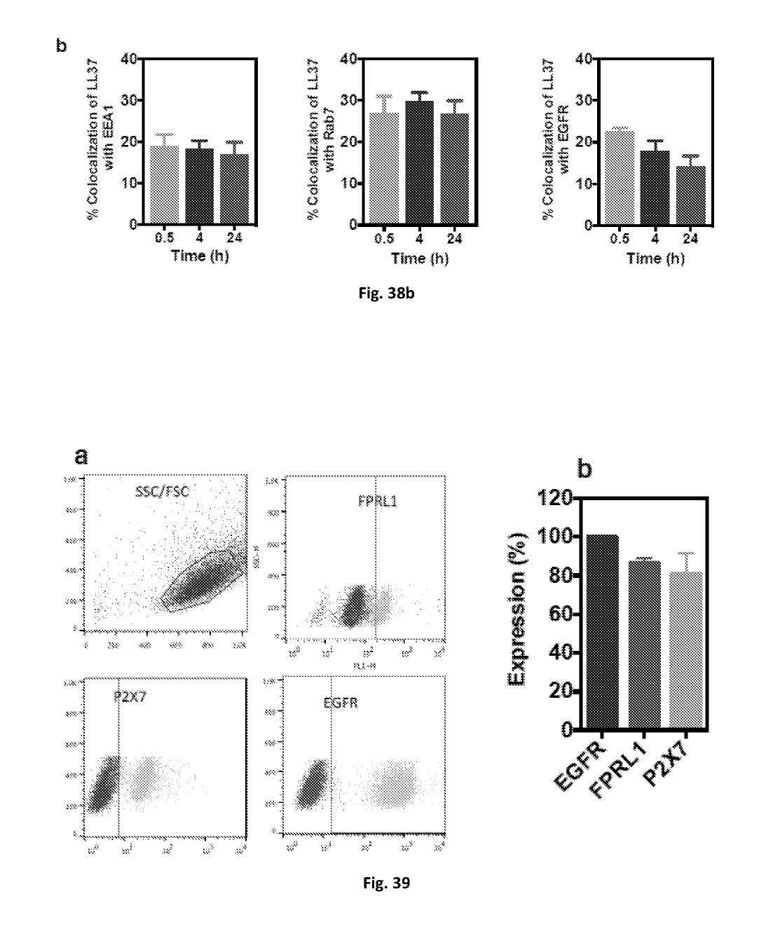

In an embodiment, it was examined the intracellular trafficking of rhodamine-labeled LL37-Au NPs or rhodamine-labeled LL37 by confocal microscopy in keratinocytes. Images of cells reconstructed from z-stacks of confocal images indicated significant cellular uptake of NPs (FIG. 23a and FIG. 37) and LL37 (FIG. 38). Co-localization of LL37-Au NPs with EEA1 vesicles peaked at 4 h (ca. 25%) while co-localization with Rab7 peaked at 24 h (ca. 33%) (FIG. 36b). Co-localization of LL37-Au NPs with EGFR during the 24 h was also observed. LL37 was also internalized by keratinocytes and 50% of LL37 was within EEA1 and Rab7 positive endolysosomal compartments up to 24 h (FIG. 38). TEM results confirm that NPs are taken up by keratinocytes and localize within endolysosomal vesicles already after 30 min of NPs incubation (FIGS. 23b.1-23b.3); however, foci of NPs interacting with the cell membrane are still visible at 4 h.

In an embodiment, taken together, LL37-Au NPs and LL37 peptide are both internalized by keratinocytes mainly by scavenger receptors and a significant percentage (up to 50%) of both formulations accumulate in the EEA1 and Rab7-positive endolysosomal compartments. However, both LL37-Au NPs and LL37 still co-localize with cell membrane (labelled by an epidermal growth factor receptor, EGFR, antibody) between 4 and 24 h post-exposure as shown by confocal microscopy (LL37 and LL37-Au NPs) and TEM (LL37-Au NPs).

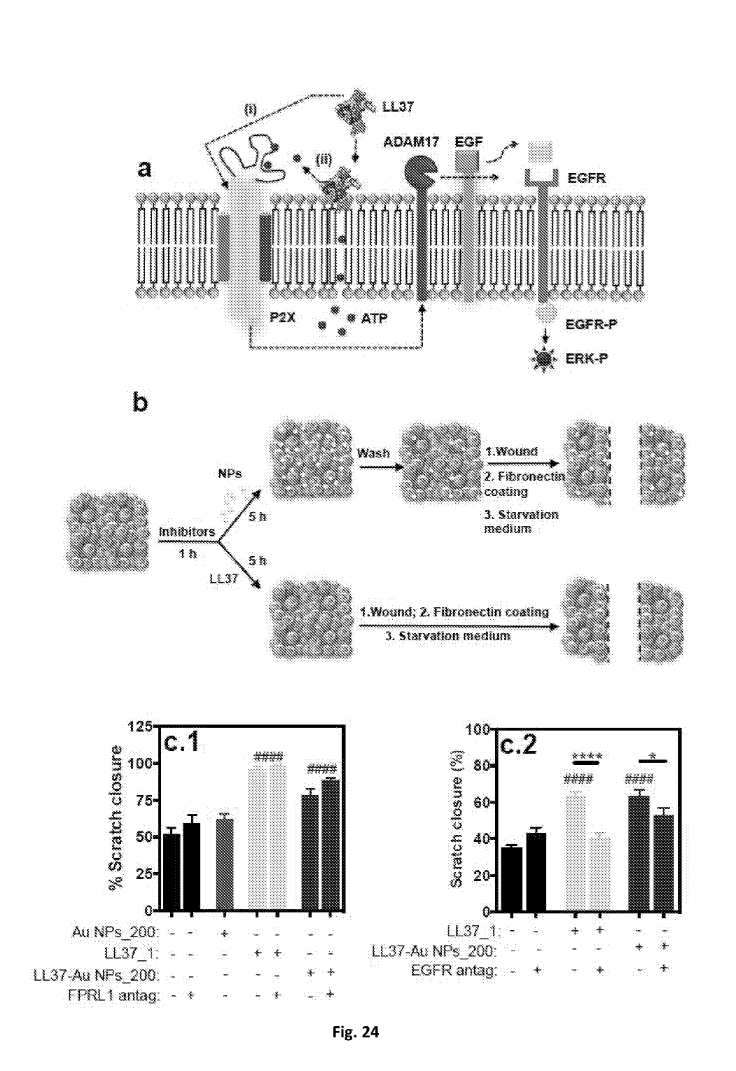

In an embodiment, both LL37-Au NPs and LL37 peptide promote keratinocyte migration through P2X, ADAM17 and EGFR. Migration of keratinocytes is an important step in skin wound healing. Previous studies have shown that LL37 activates keratinocyte migration by the transactivation of EGFR (FIG. 24a). This process involves the activation of metalloproteinases (likely ADAM10 and/or ADAM17) that releases EGF anchored to cell membrane into a heparin-binding EGF (HB-EGF), which in turn binds to EGFR. This leads to the phosphorylation of ERK1/2 and STAT3, translocation of STAT3 into the nucleus and finally the initiation of the transcription of target genes. So far little is known about how this cascade is initiated. Therefore, it was studied the pro-migratory properties of LL37 peptide, LL37-Au NPs and Au NPs by an in vitro scratch assay in keratinocytes for 72 h (FIG. 24b). Initially, the expression of putative receptors of LL37 in keratinocytes was evaluated by flow cytometry. Keratinocytes expressed high levels of EGFR, FPRL-1, a receptor that has been described to mediate the bioactivity of LL37 in endothelial cells, and P2X7, a receptor that has been described to mediate the bioactivity of LL37 in fibroblasts and monocytes (FIG. 39). Next, it was evaluated whether the migration of keratinocytes was due to the transactivation of EGFR. Both LL37 peptide and LL37-Au NPs induced the migration of keratinocytes. The inhibition of FPRL-1 by the antagonist WRW4³³ had no measurable effect in the migration of keratinocytes; however, the inhibition of EGFR by Erlotinib decreased significantly the migration of keratinocytes treated with LL37 peptide or LL37-Au NPs (FIGS. 24c.1 and 24c.2).

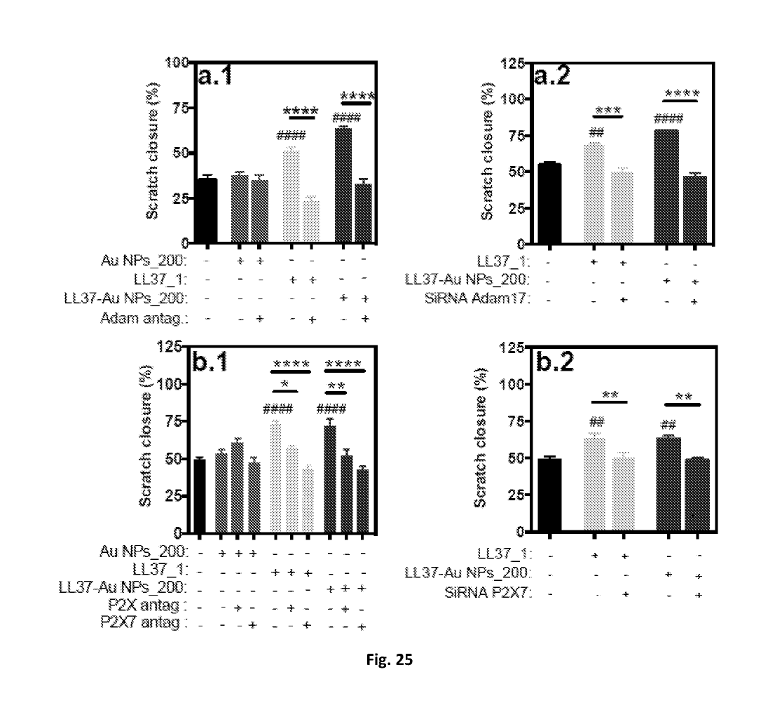

In an embodiment, to show the involvement of metalloproteases in the transactivation of EGFR it was have performed the scratch assay in the presence of Marimastat, a general ADAM inhibitor (FIG. 25a.1). The pro-migratory properties of both LL37 and LL37-Au NPs significantly decreased in the presence of the ADAM inhibitor. Because studies have shown the involvement of ADAM17 in the transactivation of EGFR³⁰ it was performed knockdown studies for ADAM17 using siRNA (FIG. 25a.2). The results clearly show that the inhibition of ADAM17 decreased the migratory properties of keratinocytes after activation by LL37 or LL37-Au NPs.

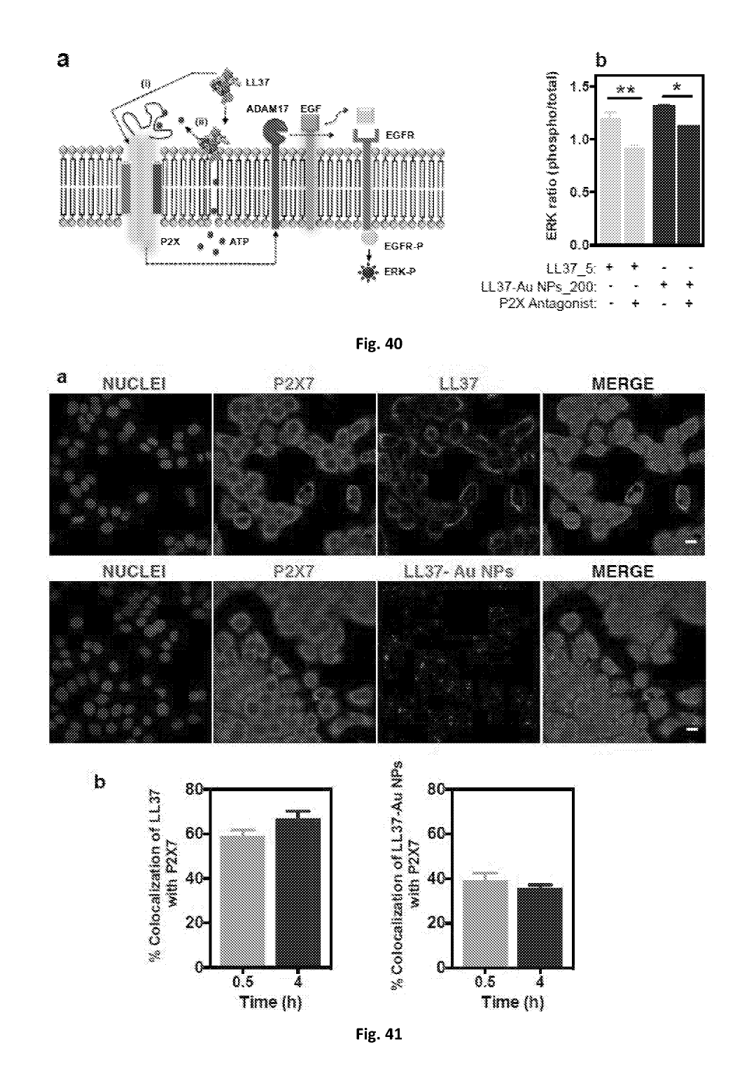

Recent studies have suggested that LL37 binds non-specifically to purinergic (P2X) receptors on the basis of their cationic or hydrophobic properties; however, no experimental evidence has been reported so far. To show the involvement of purinergic receptors in the transactivation of EGFR it was performed the scratch assay in the presence of pyridoxalphosphate-6-azophenyl-2′,4′-disulfonic acid tetrasodium salt (PPADS), a broad-spectrum purinergic receptor antagonist (FIG. 25b.1). The results show that keratinocyte migration was inhibited after activation with LL37 or LL37-Au NPs. In addition, PPADS treatment decreased significantly the phosphorylation of ERK1/2 a downstream target of EGFR transactivation (FIG. 40b). Because it has been hypothesized recently that LL37 binds non-specifically to P2X7 receptors, we inhibited chemically the receptor by A-740003 (FIG. 25b.1) or siRNA (FIG. 25b.2). In both cases, keratinocyte migration was inhibited after activation with LL37 or LL37-Au NPs. Moreover, immunocytochemistry analyses show co-localization of LL37-Au NPs or LL37 with P2X7 (FIG. 41).

In an embodiment and to further confirm the involvement of P2X receptors in the LL37-Au NPs and LL37 activity in keratinocytes it was performed electrophysiological recordings. In whole-cell patch-clamped keratinocytes at a holding potential of −70 mV, the exposure to either LL37 peptide, LL37-Au NPs or Au NPs elicited an inward current (FIG. 26a). It was next attempted to evaluate if the observed inward currents were mediated by ATP-gated P2X channels. For that purpose, it was evaluated the impact of apyrase, an enzyme that converts ATP into AMP, to the inward-currents elicited by the different compounds. It was observed that the presence of apyrase reduced the charge transfer (Q) triggered by Au NPs and LL37-Au NPs but not by LL37. Whole-cell patch-clamp recordings showed that LL37-Au NPs (FIG. 26a.3) or Au NPs (FIG. 26 a.1) triggered the release of extracellular ATP from keratinocytes, which in turn activated P2X receptors. If cells were treated with apyrase after the pulse with LL37-Au NPs or Au NPs there was a significant decrease in the inward electric current. In contrast, whole-cell patch-clamp recordings showed that LL37 peptide (FIG. 26a.2) did not trigger the release of extracellular ATP because no decrease in the inward electric current was observed after treatment with apyrase (FIG. 26a.1 and FIG. 26a.2).

In an embodiment, the results indicate that LL37 peptide and LL37-Au NPs promote keratinocyte migration by the transactivation of EGFR. The process in both cases is likely initiated at the P2X7 receptors, as confirmed by chemical and genetic inhibition studies. The activation of P2X receptors by LL37-Au NPs, but not by LL37 peptide, involves the release of extracellular ATP.

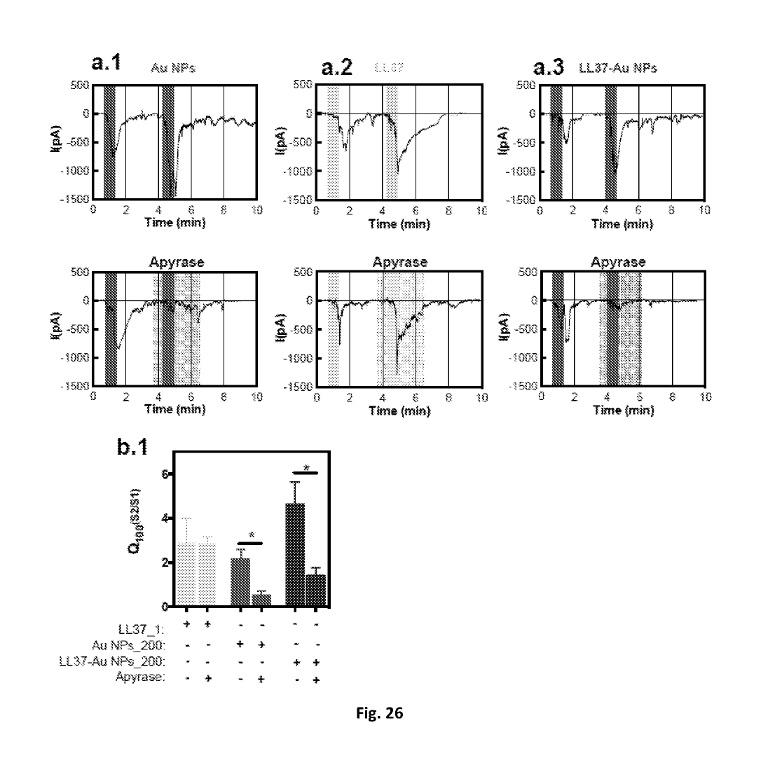

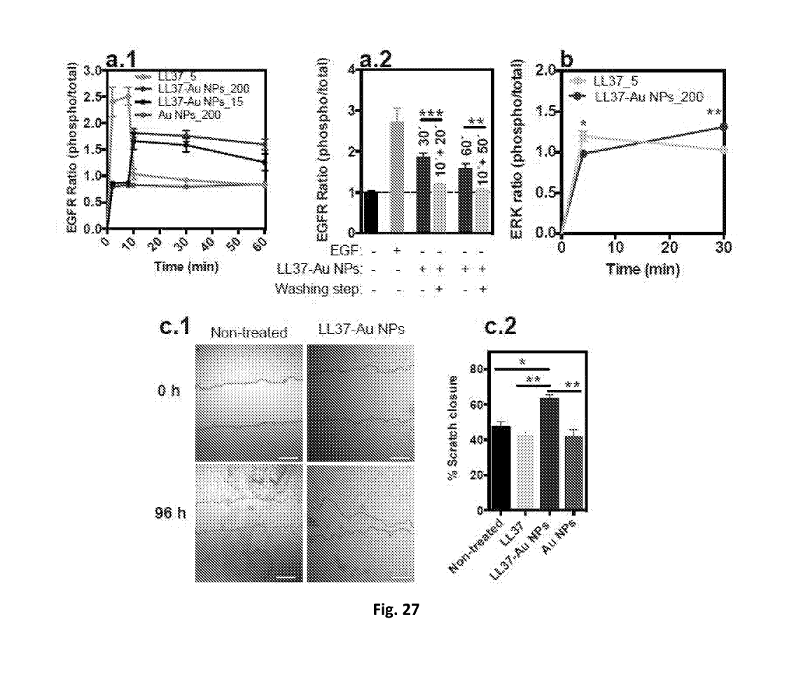

In an embodiment, LL37-Au NPs prolong EGFR and ERK phosporylation and keratinocyte migration compared to LL37 peptide. Epithelial wounding induces activation of EGFR and its two major downstream effectors PI3K and extracellular signal-regulated kinase ERK. It was evaluated the phosphorylation of EGFR in keratinocytes mediated by LL37 peptide and LL37-Au NPs. In line with previous results, the phosphorylation of EGFR by LL37 peptide was rapid (peaked at 8 min) and persisted for 10 min (FIG. 27 a.1). Interestingly, the phosphorylation of EGFR by LL37-Au NPs peaked at 10 min of contact and persisted for at least 60 min. The phosphorylation level was not significantly affected using a lower concentration of LL37-Au NPs (15 μg/mL). In addition, LL37-Au NPs lose their capacity to prolong the phosphorylation of EGFR in keratinocytes if washed after 10 min of contact, indicating that EGFR phosphorylation is dependent on NP-cell contact time (FIG. 27 a.2).

In an embodiment, it was also examined whether the prolonged phosphorylation of EGFR was correlated with a prolonged phosphorylation of ERK1/2. The phosphorylation of ERK1/2 in keratinocytes by LL37 peptide peaked at 4 min and then decreased at time 30 min, while the phosphorylation of ERK1/2 in cells by LL37-Au NPs peaked at 30 min (FIG. 27b). Thus, the results confirm a correlation between EGFR and ERK1/2 phosphorylation.

In an embodiment, to evaluate the functional impact of the prolonged phosphorylation of EGFR it was conducted a scratch assay in which the wound was large enough to evaluate the long-term migratory activity of keratinocytes. Keratinocytes treated with LL37-Au NPs had significantly higher migratory capacity than cells activated with LL37 or the other controls (FIGS. 27 c.1 and 27 c.2).

In an embodiment, taken together, the results show that LL37-Au NPs but not LL37 peptide have the capacity to prolong the phosphorylation of EGFR and ERK1/2 (from few minutes up to 1 h) and thus enhancing the migratory properties of keratinocytes in a large in vitro wound model.

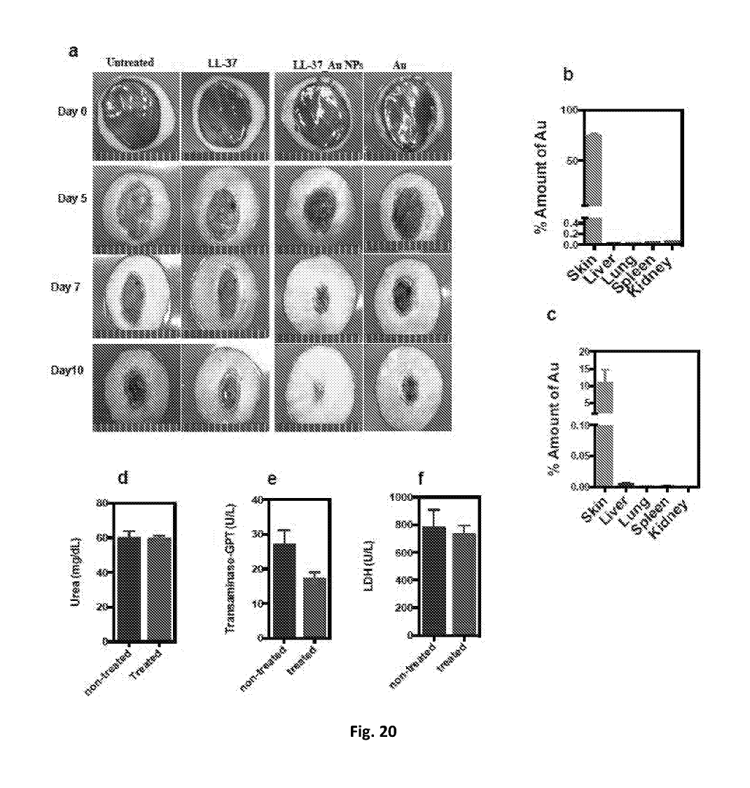

In an embodiment, the LL37-Au NPs have higher in vivo wound healing activity than LL37 peptide. To further verify the biological effects of LL37-Au NPs wound healing experiments were performed in a splinted mouse full thickness excisional model. The freshly created wounds were immediately treated with LL37 peptide, LL37-Au NPs, Au NPs or non-treated (control). Wound area was monitored over a period of 10 days. At day 5, LL37-Au NP-treated mice showed an acceleration of wound healing as compared to the control (FIG. 7a and FIG. 20a). LL37-Au NP-treated mice showed complete wound healing after 10 days, whereas LL37 peptide-treated mice showed 70% healing after 10 days. No adverse effects on body weight, general health, or behaviour of the mice was observed after NP treatment. The effect of LL37-Au NPs on wound healing was assessed by histological and immunohystochemical examination of epithelial gap closure. Skin sections were stained with hematoxylin and eosin (H&E) for general observation of skin layers and the extent of collagen deposition in healed tissue was determined by Masson's Trichrome (MT) staining. On day 5, the thick scab and the epithelium layer were formed in wounds treated with LL37-Au NPs compared to LL37 and Au NP-treated wounds (FIG. 7e). In addition, the wounds treated with LL37-Au NPs have higher levels of collagen than the ones treated with LL37 and Au NP-treated wounds, as shown by Sircol quantification (FIG. 7c). At day 10, a prominent thick epithelium layer was developed in LL37-Au NPs group and wound re-epithelization and deposition of connective tissue processes were mostly completed, leading to closure of wound (FIG. 7e).

In an embodiment, it has been shown that IL6 is involved in wound healing by regulating leukocyte infiltration, angiogenesis, collagen accumulation and LL37-mediated keratinocyte migration. Epidermal keratinocytes have been identified as the main source of IL6 production in the skin and several host defence peptides including LL37 have been shown to stimulate IL6 expression^{41, 42}. Therefore, it was evaluated the expression of IL6 in the wounds of all the experimental groups by qRT-PCR. Wounds treated with LL37-Au NPs had higher expression of IL6 than wounds treated with LL37 or Au NPs (FIG. 7d). These studies were extended to quantify neutrophil infiltration in wounds by myeloperoxidase (MPO) analysis. MPO is an enzyme that is found predominantly in the azurophilic granules of neutrophils and can be used as a quantitative index of inflammatory infiltration. MPO activity in wound tissue was significantly decreased after treatment with LL37 peptide and LL37-Au NPs on day 10, demonstrating anti-inflammatory properties of LL37 peptide (FIG. 7b). No reduction in MPO activity was found in wound tissue treated with Au NPs.

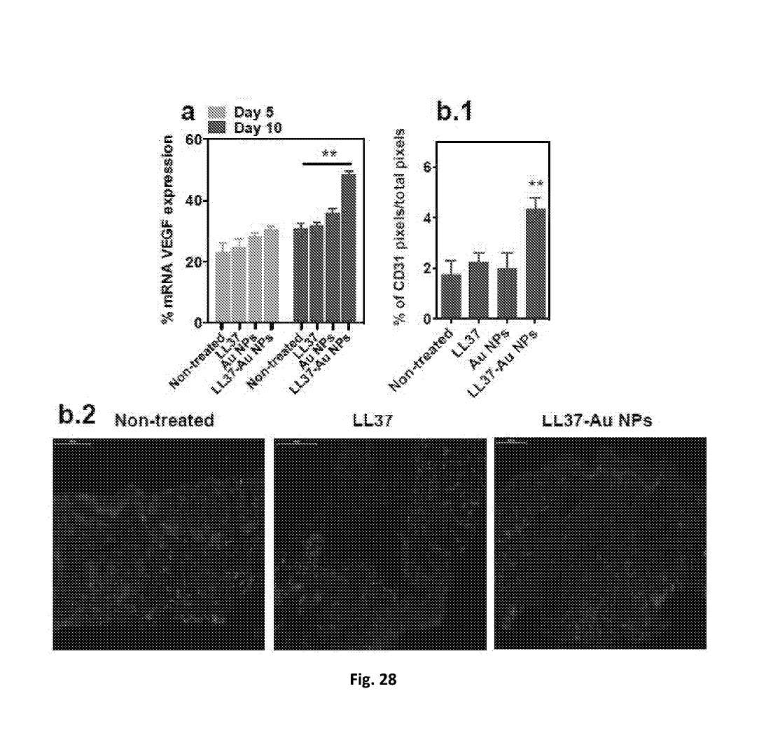

In an embodiment, the impact of LL37 and LL37-Au NPs in the vascularization of the wounds was assessed. The levels of VEGF were quantified by qRT-PCR and expressed as percentage relatively to control (FIG. 28a). The results show that wounds treated with LL37-Au NPs have higher expression of VEGF at day 10 than wounds treated with LL37 or Au NPs. Similarly, wounds treated with LL37-Au NPs have higher levels of CD31 than wounds treated with LL37 or Au NPs at day 10 (FIG. 28b).

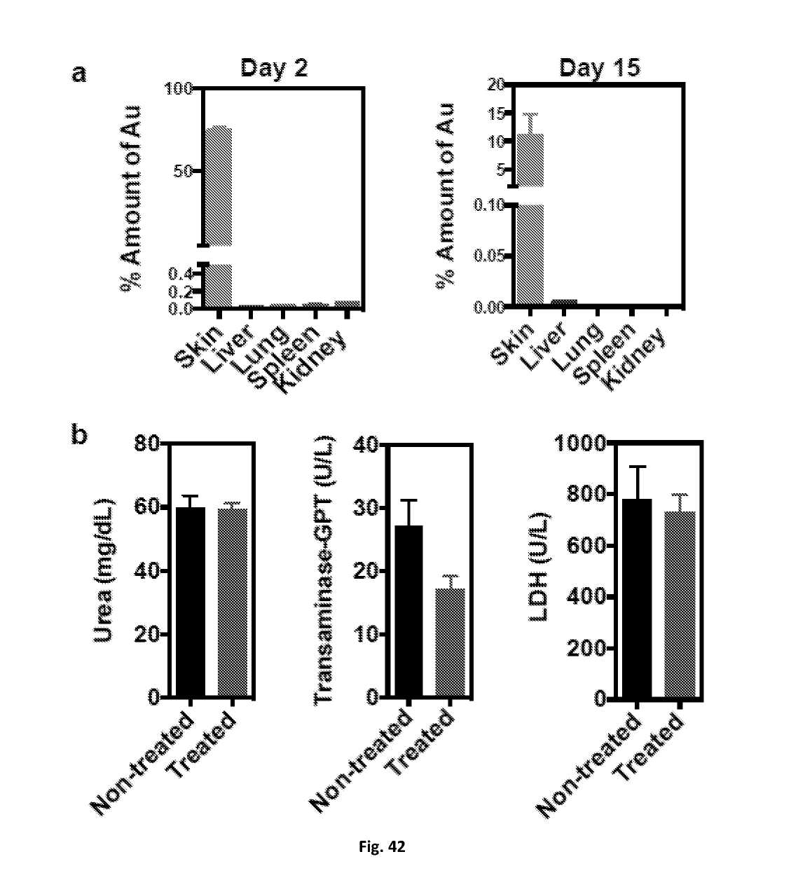

In an embodiment, the interactions between skin and colloidal Au NPs of different physicochemical characteristics have been previously investigated. It was decided to investigate the accumulation of LL37-Au NPs in the skin after topical administration and in the main organs of the mouse full thickness excisional model. LL37-Au NPs accumulated in the skin, being 74% of the initial concentration of the NPs found in the skin at day 2; however, only 10% of the initial concentration of NPs was found at day 15 (FIG. 42a). Less than 1% of the topical dose was found in the main organs at days 2 and 15. The toxic effects of the LL37-Au NPs against liver (transaminase-GPT), kidney (urea), lung and general damage (lactate dehydrogenase) were also measured after 48 h of exposure (FIG. 42b). The topical administration of the LL37-Au NPs did not elevate the levels of these key parameters, indicating that the NPs did not cause significant damage to organ functions at the tested doses.

In an embodiment, the results show that LL37-Au NPs have higher wound healing activity than LL37 peptide. The results further show that LL37-Au NPs are eliminated from the skin.

In an embodiment, it is shown that LL37-conjugated NPs have higher in vitro (pro-migratory properties against keratinocytes) and in vivo (skin wound healing) bioactivity than soluble LL37. The results further indicate that LL37-Au NPs activate P2X7 receptor followed by a prolonged transactivation of EGFR and enhanced migratory properties of keratinocytes in an in vitro wound model.

Several nanomedicine-based therapies have been reported in the last few years for wound healing; however, very few formulations combined antimicrobial with pro-regenerative properties. This is very important since many chronic wounds get infected during healing and thus the combination of both properties in the same therapeutic formulation is very beneficial. In addition, very few formulations have been designed to take advantage of the chemical conjugation of the bioactive agent (e.g. proteins, siRNAs, peptides) into the surface of the NP and thus acting as multivalent ligands. Indeed, the binding and activation of membrane receptors might depend in the ligand multivalency and controlled spacing of the multivalent ligands. The functional data now disclosed shows that LL37 peptide and LL37-Au NPs activate keratinotyces by P2X7 receptors followed by the activation of ADAM17 and the transactivation of EGFR. Keratinocytes inhibited by general purinergic receptor antagonists or by specific P2X7 receptor inhibitors (chemical or genetic) have decreased migration after activation by LL37 peptide or LL37-Au NPs. The electrophysiology results suggest that the activation of P2X receptors by LL37 does not involve extracellular ATP. Indeed recent experimental results in mononuclear cells suggest that the biological role of LL37 is likely mediated by a complex between LL37 and P2X7R. P2X7R forms a large multimolecular complex with several proteins in the plasma membrane such as 1-actin, integrin 12, heat shock proteins and non-muscle myosin. Moreover, the direct or indirect (by one of the co-associated molecules) activation of P2X7R can trigger PLD, MAPK- or PI3K-mediated downstream signaling pathways. From the results and data in the literature is still unclear whether there is a specific domain(s) of P2X7R or some co-associated adhesion molecule that mediates the binding of LL37. Importantly, in case of LL37-Au NPs, the activation of P2X receptors seems to be mediated in part by extracellular ATP. Cell treatment with apyrase decreased significantly, but not all, the inward electric current. Because Au NPs also induce the release of extracellular ATP but have no significant effect in the migratory properties of keratinocytes it is likely that the activation mechanism of LL37-Au NPs might involve other mechanisms than extracellular ATP. Therefore, it is speculate that the activation of P2X receptors by LL37-Au NPs is due to a combinatorial effect by extracellular ATP and the direct/indirect interaction of LL37 of the surface of the NP with P2X7R.

This is the first disclosure documenting the long-lasting phosphorylation of keratinocytes activated through purinergic receptors. The prolongation of EGFR phosphorylation may be explained by (i) an increased recycling rate of EGFR or (ii) retention of the phosphorylated EGFR at the plasma membrane preventing EGFR desphosphorylation. Prolongation of EGFR phosphorylation has been described in some studies associated with the prevention of EGFR desphosphorylation. For example, the association of EGFR with the receptor erbB2, a major interaction partner and coactivator of EGFR, is sufficient to prolong and enhance the net phosphorylation of EGFR⁵¹. CTEN, a focal adhesion molecule of tensin family, is also able to extend the life of phosphorylated EGFR by decreasing EGFR reduction though the decrease of EGFR ubiquitination. Moreover, EGF immobilized in two-dimensional surfaces is able to prolong the phosphorylation of EGFR. These results suggest that the prolongation of EGFR phosphorylation by LL37-Au NPs might be mediated by the retention of the phosphorylated EGFR at the plasma membrane preventing EGFR desphosphorylation. It was observed the co-localization of LL37-Au NPs with P2X7 and EGFR up to 4 h of contact by confocal microscopy analyses and it was observed the interaction of the NPs with the cell membrane until the 4 h of contact by TEM analyses. However, the experimental results cannot rule out the possibility that the prolongation of EGFR may also be mediated during NP internalization.

In an embodiment, LL37-Au NPs have higher wound healing activity than LL37 peptide. The wound healing mechanism of LL37-Au NPs is mediated by an increased IL6 production, decreased inflammation (as evaluated by myeloperoxidase activity) and increased neovascularization. Although this disclosure is the first study to demonstrate the in vivo wound healing properties of immobilized LL37, the in vivo wound healing properties of LL37 peptide have been previously demonstrated in animal models and in phase I/II clinical trials. The primary safety and tolerability end-point for LL37 was met for patients with venous leg ulcers. The results showed that patients treated with LL37 (twice per week; 0.5 mg/mL) had a statistically significant improved healing rate compared with placebo. However, the peptide was administered twice per week and thus LL37-Au NPs may offer an alternative approach to extend the bioactivity of the peptide in a single administration. Recently, it was reported a delivery system of LL37 based on the encapsulation of the peptide in poly(lactic-co-glycolic acid) nanoparticles. It has been shown that the sustained release of LL37 could accelerate wound healing; yet the regenerative profile was more discrete than the one observed in this study. It is possible that the superior wound healing properties of LL37-Au NPs to LL37 might involve different cells of the skin and not only keratinocytes.

In an embodiment, the formulation reported here has an Au core and a hydrophilic cationic peptide shell, and makes use of an innovative one-step synthetic scheme. It were selected Au NPs because it was relatively easy to control their size while presenting a high density of LL37 in their surface and because of their history in the context of wound healing. The in vivo biodistribution studies showed that most of the NPs (90%) are eliminated from the skin at day 15 post-application (therefore 5 days after wound closure) and residual (less than 0.05%) amount of NPs are found in the major organs. Although further tests are needed to evaluate the in vivo accumulation of NPs the results now disclosed show that most of the NPs are eliminated from the skin.

In an embodiment it is reported an AMP-nanoscale therapeutic formulation with high skin regenerative potential, obtained in a rapid one-step synthetic process. LL37-Au NPs have higher in vitro (keratinocyte migration assay) and in vivo (skin wound healing assay) bioactivity than soluble LL37 peptide. The enhanced activity of LL37-Au NPs relatively to LL37 peptide is due to a prolonged activation of EGFR in keratinocytes, likely due to the retention of the phosphorylated EGFR at the plasma membrane preventing EGFR dephosphorylating. The results presented here are an exciting first step toward the development of AMP-based nanotherapeutics for skin disorders paving the way for additional studies in more complex animal models.

### Materials and Methods

In an embodiment, the materials were: HAuCl₄.3H₂O, Na₃C₆H₅O₇ and HEPES, all acquired to Sigma-Aldrich, were used as received. Lyophilized LL37 peptide modified (SEQ. ID. 7) with a C-terminal cysteine (LLGDFFRKSKEKIGKEFKRIVQRIKDFLRNLVPRTESC) was purchased from Caslo Laboratory, Denmark. The peptide was synthesized by conventional solid-phase synthesis, purified by high performance liquid chromatography, and characterized by matrix assisted laser desorption ionization time-of-flight (MALDI-TOF) mass spectroscopy. The purity of the peptide was 96%. Rhodamine B isothiocyanate and HEPES were purchased from Sigma.

In an embodiment, NP were prepared as follows: LL37 (0.5 mM) were dissolved initially in DMF (100 μL) followed by addition of HEPES (900 μL, 100 mM, pH 5). HAuCl₄.3H₂O (10⁻² M, 50 μL) was added to a peptide solution (0.25 mM, 950 mL; therefore the final concentration of HAuCl₄ was 0.5 mM) and the NP synthesis was carried out at 25° C. LL37-Au NPs were also synthesized using HAuCl₄.3H₂O (final concentration 0.25 mM, 0.5 mM and 1 mM), and HEPES (100 mM, pH 6 and pH 7.5) by the same procedure at 25° C. The synthesized Au NPs were centrifuged at 14,000 rpm for 20 min at 4° C. followed by washing with Milli-Q water to remove unreacted peptides and HEPES, frozen and freeze-dried at 223 K using a Snijders Scientific freeze-dryer. Spherical Au NPs were also synthesized via citrate reduction method. An aqueous HAuCl₄ solution (0.5 mM, 100 mL of water) was boiled in a 250 mL round bottom flask while being stirred after which an aqueous sodium citrate solution (2%, w/v, in water) was added. The reaction was allowed to run until the solution reached a wine red color, indicating the reaction was completed. Fluorescent Au NPs and LL37-Au NPs were prepared by addition of DMSO (0.5 mM) solution of rhodamine to achieve a final concentration of 25 μM for flow cytometry and confocal microscopy studies. Free rhodamine molecules in the colloidal gold solution were removed by centrifugation at 12,000 rpm for 15 min at 4° C. followed by one washings with Milli-Q water. The pellet obtained after centrifugation was redispersed in Milli-Q water and then dialysed. The methods for NP characterization are presented in the Supplementary Information.

In an embodiment, human keratinocyte cell line (HaCaT cell line, CLS, Eppelheim, Germany) was cultured as recommended by the vendor. Briefly, cells were cultivated using DMEM supplemented with 1% (v/v) penicillin and streptomycin (Invitrogen) and 10% (v/v) fetal bovine serum (FBS, Invitrogen) until 90% of confluence. For passage, keratinocytes were initially trypsinized and then scraped. The cells were sub-cultured at a ratio of 1:3 until achieving the number of cells required for the experiment. The methods to evaluate the cytotoxicity, internalization and intracellular trafficking of LL37-Au NPs and LL37 peptide are described in detail in the supplementary information.

In an embodiment, the NP bioactivity was carried out by scratch assay. Keratinocytes (passage 35-40) were seeded at a density of 2×10⁴ cells/well in fibronectin-coated 96-well plate in DMEM supplemented with 1% (v/v) penicillin and streptomycin and 10% (v/v) FBS. After 48 h, cells were initially starved for 15 h in DMEM with 0.5% FBS, inactivated with mitomycin (5 μg/mL) for 2 h, and then incubated with LL37 (1 μg/mL), LL37-Au NPs or Au NPs (both at 200 μg/mL) for 5 h at 37° C. and 5% CO₂. In case of chemical inhibition, the chemical inhibitors for FPRL1 (WRW4, Calbiochem, 10 μM), EGFR (Erlotinib HCL, OSI-744, Selleck Chemicals, 2 nM), ADAM17 (Marimastat, Sigma, 10 μM), P2X (PPADS, Sigma, 100 μM), for P2X7 (A-740003, Sigma, at a concentration of 500 nM), were added to the cells 1 h before the incubation with LL37 peptide and LL37-Au NPs and then maintained during the assay. All inhibitors used in the present disclosure were tested, and none of them showed any significant toxicity to cells under our experimental conditions (data not shown). After the 5 h treatment, cells were washed twice with PBS to remove non-internalized NPs and a scratch was created with a sterile pipette tip. The detached cells were washed twice with PBS and then plates were re-coated with fibronectin (10 μg/mL in starvation medium) for 1 h at 37° C. Cells were washed and maintained in starvation medium up to 72 h. Cell migration was monitored overtime by a in Cell Microscope 2000 (GE Healthcare) (objective 2×). The cells treated with LL37 peptide were cultured with starvation medium containing LL37 for the entire duration of the experiment. Scratch areas were quantified using the AxioVision software (Carl Zeiss). Wound areas were normalized by the initial area (n=6 images). In case of siRNA knock-down studies, on-target plus human ADAM17 siRNA or P2X7R siRNA (both from Dharmacon) were used to silence ADAM17 or P2X7 before performing the scratch assay. Keratinocytes were transfected with 50 nM siRNA using 0.25 μL of Lipofectamine RNAiMAX (Life Technologies) for 24 h in antibiotic-free complete medium before starting the scratch assay.

In an embodiment, to test the prolonged effect of LL37-Au NPs on keratinocyte migration, cells were plated in fibronectin-coated 24-well plate (1×10⁵ cells/well), after 48 h cells were starved for 15 h in DMEM with 0.5% FBS and then the scratch was made with a sterile pipette tip. Only after this step, the cells were incubated with LL37 (1 μg/mL), LL37-Au NPs and Au NPs (15 μg/mL) in starvation medium up to 96 h. During this time, cell migration was monitored by scratch area quantification.

In an embodiment, the NP bioactivity was carried out as follows: electrophysiological recordings. Whole-cell patch-clamp recordings of HaCaT cells were performed using borosilicate glass patch pipettes with a resistance of 2-5 MΩ and compensating the series resistance by 50-60% with a List EPC-7 amplifier, as described previously⁵⁶. The pipettes were filled with (in mM): 135 K-gluconate, 10 CsCl, 0.4 NaH₂PO₄, 0.73 CaCl₂, 1 MgCl₂, 1 EGTA, and 14 HEPES (pH 7.4). The external solution contained (mM): 140 NaCl, 3 KCl, 2 CaCl₂, 1 MgCl₂, 15 HEPES and 10 glucose (pH 7.4). Cells were rapidly perfused as previously described and all experiments were carried out at RT (22-25° C.). The currents were recorded at −70 mV, filtered (2-pole Butterworth filter, −12 dB/octave) and digitized to a personal computer at a sampling rate of 1 kHz for analysis using pCLAMP software (AXON instruments). Charge transfer was calculated by the integration of the area under the current in the first 100 s (Q100) from the beginning of the exposure to Au NPs, LL37 or LL37-Au NPs in the absence or presence of apyrase (darker background; 20 U/mL; Sigma).

In an embodiment, the in vivo wound healing and tissue collection. 6-7 week old RjHan:NMRI female mice (Janvier, BE) were anesthesized with isoflurane and the dorsal area was shaved using a depilatory cream a day before the surgery. Two 0.5-mm-thick silicone (Grace biolabs, UK) donut-shaped splints (OD=20 mm, ID=10 mm) were fixed on either side of the dorsal midline, approximately 3.5 cm from the ears and positioned with 6-0 nylon sutures (Monosof, USA). Full-thickness excisional wounds were made using an 8 mm round skin biopsy punch (Kai Europe GMBH, DE), centered within each splint. 10 mice were randomly assigned per group and were administered intradermally at several sites around the wound with only vehicle (0.9% w/v NaCl, Mini-Plasco, BE), LL37 (70 fig), LL37-Au NPs (200 fig) and Au NPs (200 fig) in autoclaved water by sterile insulin syringe (BD medical, France) as a dispersion in 30 μl of vehicle. Two wounds were made on each mouse to increase sample size and to avoid cross-contamination both wounds were administered with the same treatment. Thus, n=10 animals (20 wounds) per group. Wounds were covered with transparent sterile adhesive bandage (IV3000, Smith & Nephew, UK) followed by adhesive fabric tape (BSN medical, France) to prevent the chewing of splints by mice. On days 0, 2, 5, 7, 10, 13 and 16 wounds were digitally photographed by Leica IC80 HD camera (Leica, Swiss). Optical zoom was maintained identical throughout the experiments. Wound areas were quantified using the Jmicro Vision software developed by N. Roduit at University of Geneva, Switzerland. Wound sizes are expressed as percentage of the initial respective wound. On day 5 and 10, three animals per group were sacrificed and wounds along with surrounding tissue were collected for further experiments and bisected into two halves. Remaining animals were sacrificed on day 16. The animal studies were approved by the animal care and ethical committee of health science sector, Université Catholique de Louvain. The methods for histology, immunohistochemistry, collagen quantification, MPO activity and gene analyses are provided in Supplementary Information.

Statistical Analysis. The data are presented as mean±SD and results were considered significant when P<0.05 (*), P<0.01 (**), or P<0.001 (***). Statistical analysis performed by using one-way ANOVA with the Bonferroni test applied post hoc for paired comparisons of means (GraphPad Prism 5.0 software).

The disclosure should not be seen in any way restricted to the embodiments described and a person with ordinary skill in the art will foresee many possibilities to modifications thereof.

The above described embodiments are combinable. The following claims further set out particular embodiments of the disclosure.

1. A composition for use in medicine or veterinary comprising a gold nanoparticle and a plurality of LL-37 peptides as antimicrobial peptide, wherein the plurality of LL-37 peptides is bound to the gold nanoparticle surface.

2. A composition according to the previous claim use in a method of treating or in therapy of wounds, or ischemic diseases, or vascular diseases, or skin disorders.

3. The composition according to any one of the previous claims wherein the LL-37 is bound to the gold nanoparticle surface by covalent bonds.

4. The composition according to any one of the previous claims further comprising a buffer.

5. The composition according to any one of the previous claims wherein the pH of the composition is between 4.5-8, preferably 5-7.5.

6. The composition according to any one of the previous claims wherein the pH of the composition is between 5-6.

7. The composition according to any one of the previous claims 4-6 wherein the buffer is selected from the following list: TAPS, Bicine, Tris, Tricine, TAPSO, HEPES, TES, MOPS, PIPES, Cacodylate, SSC, MES, or combinations thereof.

8. The composition according to any one of the previous claims wherein the ratio LL-37:gold nanoparticle is 3:1-1:2, preferably 2:1-1:1.

9. The composition according to any one of the previous claims wherein the antimicrobial peptide concentration is between 0.20-0.10 mM, preferentially between 0.3-0.5 mM.

10. The composition according to any one of the previous claims further comprising stem cells.

11. The composition according to any one of the previous claims further comprising endothelial cells, namely human umbilical vein endothelial cells, or keratinocytes, or combinations thereof.

12. The composition according to any one of the previous claims, wherein the composition is a topic formulation or an injectable formulation.

13. The composition according to any one of the previous claims, for the use in the treatment of wounds in diabetic mammals.

14. The composition according to any one of the previous claims for the use in the treatment of eczema or psoriasis.

15. Use of the composition as described in any one of the previous claims as an enhancer in the treatment of wound healing.
